(19) United States
(12) Patent Application Publication
(10) Pub. No.: US 2025/0072282 A1
(43) Pub. Date: Feb. 27, 2025

(54) ANTHRACENE DERIVATIVE COMPOUND AND ORGANIC LIGHT-EMITTING DEVICE COMPRISING SAME

(71) Applicant: SFC CO., LTD., Cheongju-si (KR)

(72) Inventors: Jin-hwi CHO, Cheongju-si (KR); Se-jin YU, Cheongju-si (KR); So-young SHIM, Cheongju-si (KR); Yong-woon YANG, Cheongju-si (KR)

(73) Assignee: SFC CO., LTD., Cheongju-si (KR)

(21) Appl. No.: 18/724,329

(22) PCT Filed: Dec. 29, 2022
(86) PCT No.: PCT/KR2022/021573

§ 371 Date: Jun. 26, 2024

(30) Foreign Application Priority Data
Dec. 29, 2021 (KR) 10-2021-0191419
Dec. 28, 2022 (KR) 10-2022-0186775

(51) Int. Cl. H10K 85/60; C07C 15/20; C07C 43/275; C07C 255/52; C07D 209/86; C07D 213/16; C07D 215/06; C07D 241/42; C07D 265/38; C07D 279/22; C07D 307/77; C07D 333/76; C07D 407/10; C07D 409/14; C07F 7/08; C07F 7/30; C09K 11/02; C09K 11/06; H10K 50/12

(52) U.S. Cl. CPC H10K 85/626; C07C 15/20; C07C 43/275; C07C 255/52; C07D 209/86; C07D 213/16; C07D 215/06; C07D 241/42; C07D 265/38; C07D 279/22; C07D 307/77; C07D 333/76; C07D 407/10; C07D 409/14; C07F 7/0805; C07F 7/30; C09K 11/02; C09K 11/06; H10K 85/6574; H10K 85/658; C07B 2200/05; C07C 2602/10; C07C 2602/12; C07C 2603/24; C09K 2211/1007; C09K 2211/1014; C09K 2211/1022; H10K 50/12

(57) ABSTRACT

The present invention relates to an anthracene derivative compound having a characteristic structure in which an aliphatic aromatic mixed ring group is substituted, and to an organic light-emitting device comprising same. The present invention relates to a high-efficiency and long-lifespan organic light-emitting device having remarkably improved luminous efficiency and lifespan characteristics by adopting the anthracene derivative compound according to the present invention as a host of a light-emitting layer, and adopting a polycyclic compound having a characteristic structure as a dopant of the light-emitting layer.

### TECHNICAL FIELD

The present invention relates to an anthracene derivative having a specific structure in which an anthracene moiety is substituted with a mixed aliphatic-aromatic cyclic group and an organic light emitting device including the same. More particularly, the present invention relates to a highly efficient and long-lasting organic light emitting device which includes a light emitting layer employing the anthracene derivative as a host and a polycyclic compound with a specific structure as a dopant, achieving significantly improved luminous efficiency and life characteristics.

### BACKGROUND ART

Organic light emitting devices are self-luminous devices in which electrons injected from an electron injecting electrode (cathode) recombine with holes injected from a hole injecting electrode (anode) in a light emitting layer to form excitons, which emit light while releasing energy. Such organic light emitting devices have the advantages of low driving voltage, high luminance, large viewing angle, and short response time and can be applied to full-color light emitting flat panel displays. Due to these advantages, organic light emitting devices have received attention as next-generation light sources.

The above characteristics of organic light emitting devices are achieved by structural optimization of organic layers of the devices and are supported by stable and efficient materials for the organic layers, such as hole injecting materials, hole transport materials, hole blocking materials, light emitting materials, electron transport materials, electron injecting materials, and electron blocking materials. However, more research still needs to be done to develop structurally optimized structures of organic layers for organic light emitting devices and stable and efficient materials for organic layers of organic light emitting devices.

Particularly, for maximum efficiency in a light emitting layer, an appropriate combination of energy band gaps of a host and a dopant is required such that holes and electrons migrate to the dopant through stable electrochemical paths to form excitons.

Molecules used in organic layers during the fabrication of OLEDs pile up to form crystals and are recrystallized by heat generated during deposition. This phenomenon causes the organic layers to crack, resulting in short lifetime of the devices. Thus, there is an urgent need to develop materials having specific structures that can solve the above problem and device structures using the materials.

### DETAILED DESCRIPTION OF THE INVENTION

### Problems to be Solved by the Invention

Therefore, the present invention is intended to provide a host material for a light emitting layer of an organic light emitting device that has a specific structure in which an anthracene moiety is substituted with a mixed aliphatic-aromatic cyclic group. The present invention is also intended to provide a highly efficient and long-lasting organic light emitting device including a light emitting layer employing the host material to achieve significantly improved luminous efficiency and life characteristics.

### Means for Solving the Problems

One aspect of the present invention provides an anthracene derivative as a host compound for an organic layer (preferably a light emitting layer) of a device, represented by Formula 1:

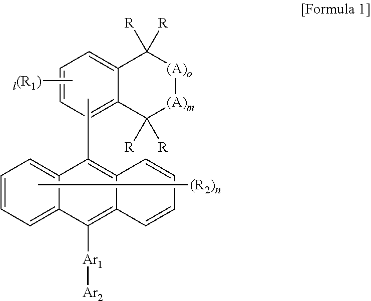

The specific structure of Formula 1, definitions of the substituents in Formula 1, and specific compounds that can be represented by Formula 1 are described below.

A further aspect of the present invention provides an organic light emitting device including a first electrode, a second electrode opposite to the first electrode, and a light emitting layer interposed between the first and second electrodes wherein the light emitting layer includes the anthracene derivative represented by Formula 1 as a host.

According to one embodiment of the present invention, the light emitting layer of the organic light emitting device may employ the anthracene derivative represented by Formula 1 as a host and a polycyclic compound represented by Formula 2 as a dopant:

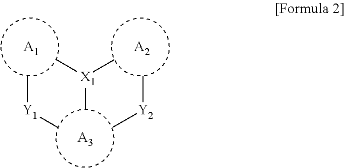

The use of the host and the dopant ensures significantly improved luminous efficiency and life characteristics of the organic light emitting device and makes the device highly efficient and long lasting.

The specific structure of Formula 2, definitions of the substituents in Formula 2, and specific compounds that can be represented by Formula 2 are described below.

### Effects of the Invention

The organic light emitting device of the present invention includes a light emitting layer employing, as a host material, the anthracene derivative having a specific structure in which an anthracene moiety is substituted with a mixed aliphatic-aromatic cyclic group. The use of the host material ensures high luminous efficiency and improved life characteristics of the device. Due to these advantages, the highly efficient and long-lasting organic light emitting device can find applications in not only lighting systems but also a variety of displays, including flat panel displays, flexible displays, and wearable displays.

### BEST MODE FOR CARRYING OUT THE INVENTION

The present invention will now be described in more detail.

One aspect of the present invention is directed to an anthracene derivative represented by Formula 1:

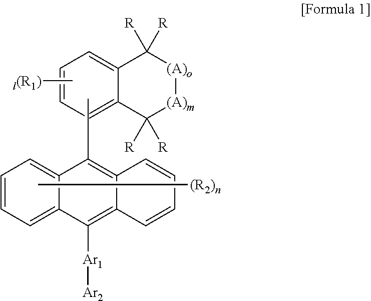

wherein the moieties A are identical to or different from each other and are each independently selected from O, S, and CR₃R₄, each of o and m is independently an integer of 1 or 2, provided that when o or m is 2 or more, the moieties A are identical to or different from each other, R, R₃, and R₄ are identical to or different from each other and are each independently selected from hydrogen, deuterium, and substituted or unsubstituted C₁-C₃₀ alkyl, with the proviso that adjacent ones of R, R₃, and R₄ are optionally linked to each other or each of R, R₃, and R₄ is optionally linked to an adjacent substituent to form an alicyclic or aromatic monocyclic or polycyclic ring, the groups R₁ are identical to or different from each other and are each independently selected from hydrogen, deuterium, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted C₃-C₅₀ mixed aliphatic-aromatic cyclic groups, 1 is an integer of 3, the groups R₁ are identical to or different from each other, with the proviso that at least one of the groups R₁ is not hydrogen or deuterium, the groups R₂ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, n is an integer of 8, the groups R₂ are identical to or different from each other, Ar₁ represents a single bond or is substituted or unsubstituted C₆-C₅₀ arylene or substituted or unsubstituted C₂-C₅₀ heteroarylene, and Ar₂ is selected from substituted or unsubstituted C₆-C₅₀ aryl, substituted or unsubstituted C₃-C₅₀ cycloalkyl, substituted or unsubstituted C₂-C₅₀ heterocycloalkyl, and substituted or unsubstituted C₂-C₅₀ heteroaryl.

The anthracene derivative is characterized in that various substituents are introduced to a specific structure in which an anthracene moiety is substituted with a mixed aliphatic-aromatic cyclic group. The anthracene derivative can be employed as a host compound in a light emitting layer of an organic light emitting device, making the organic light emitting device highly efficient and long lasting.

Generally, molecules used in organic layers during the fabrication of OLEDs pile up to form crystals and are recrystallized by heat generated during deposition. This phenomenon causes the organic layers to crack, resulting in short lifetime of the devices.

In an effort to solve the problem caused by the phenomenon of recrystallization, the present inventors have designed the anthracene derivative in which a bulky substituent introduced at a specific position in the molecule impedes regular arrangement of the molecules to lower the intermolecular bonding strength, making it difficult to form crystals. This design increases the proportion of amorphous domains to suppress a reduction in lifetime. At the same time, the introduction of the bulky substituent can prevent excitons from being quenched by 2- and 3-dimensional intermolecular interactions, achieving an increase in luminous efficiency.

The replacement of some hydrogen atoms in the compound of the present invention by deuterium atoms leads to a further increase in lifetime, which compensates for the disadvantage of short lifetime observed in the undeuterated moiety.

According to one embodiment of the present invention, at least one of the groups R₁ in Formula 1 may be substituted or unsubstituted C₆-C₂₀ aryl.

According to one embodiment of the present invention, at least four of the groups R₂ in Formula 1 may be deuterium.

The term “substituted” in the definitions of A, R, R₁, R₂, Ar₁, and Ar₂ in Formula 1 indicates substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, C₃-C₃₀ mixed aliphatic-aromatic cyclic groups, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof. The term “unsubstituted” in the same definition indicates having no substituent. One or more hydrogen atoms in each of the substituents are optionally replaced by deuterium atoms.

According to one embodiment of the present invention, the anthracene derivative of Formula 1 may be represented by Formula 1-1 or 1-2:

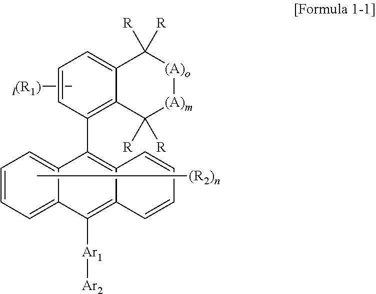

wherein R₁, R₂, Ar₁, Ar₂, A, R, l, m, n, and o are as defined in Formula 1;

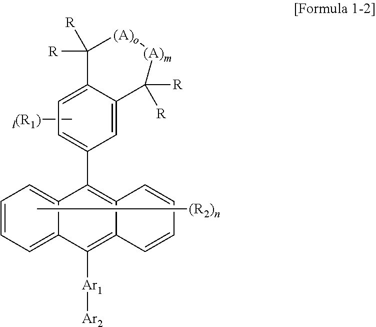

wherein R₁, R₂, Ar₁, Ar₂, A, R, l, m, n, and o are as defined in Formula 1.

According to one embodiment of the present invention, the anthracene derivative of Formula 1-1 may be represented by Formula 1-3:

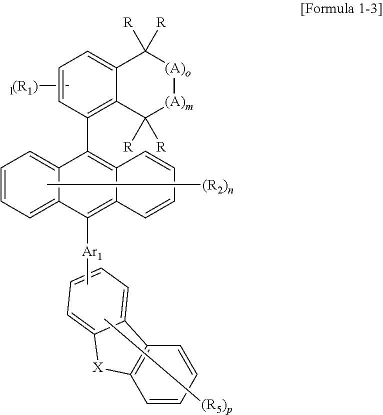

wherein X is O or S, the groups R₅ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in R₅ are optionally replaced by deuterium atoms and the adjacent R₅ groups are optionally linked to each other to form an alicyclic or aromatic monocyclic or polycyclic ring, p is an integer of 7, the groups R₅ are identical to or different from each other, and R₁, R₂, Ar₁, A, R, 1, m, n, and o are as defined in Formula 1; and the anthracene derivative of Formula 1-2 may be represented by Formula 1-4:

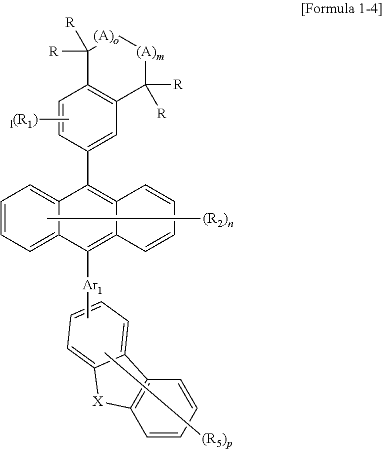

wherein X, R₅, and p are as defined in Formula 1-3 and R₁, R₂, Ar₁, A, R, l, m, n, and o are as defined in Formula 1.

A further aspect of the present invention is also directed to an organic light emitting device including a first electrode, a second electrode, and one or more organic layers interposed between the first and second electrodes wherein one of the organic layers, preferably a light emitting layer includes the anthracene derivative represented by Formula 1 as a host and a polycyclic compound represented by Formula 2 as a dopant:

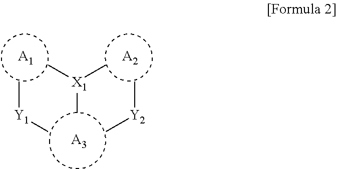

wherein X₁ is B, Y₁ and Y₂ are identical to or different from each other and are each independently selected from NR11, O, S, CR₁₂R₁₃, SiR₁₄R₁₅ or GeR₁₆R₁₇, A1 to A3 are identical to or different from each other and are each independently selected from substituted or unsubstituted C₆-C₅₀ aromatic hydrocarbon rings, substituted or unsubstituted C₃-C₅₀ aliphatic hydrocarbon rings, substituted or unsubstituted C₂-C₅₀ aromatic heterocyclic rings, substituted or unsubstituted C₂-C₅₀ aliphatic heterocyclic rings, and substituted or unsubstituted C₃-C₅₀ mixed aliphatic-aromatic cyclic group, R₁₁ to R₁₇ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, with the proviso that each of R₁₁ to R₁₇ is optionally linked to one or more of the rings A₁ to A₃ to form an alicyclic or aromatic monocyclic or polycyclic ring, R₁₂ and R₁₃ are optionally linked to each other to form an alicyclic or aromatic monocyclic or polycyclic ring, R₁₄ and R₁₅ are optionally linked to each other to form an alicyclic or aromatic monocyclic or polycyclic ring, and R₁₆ and R₁₇ are optionally linked to each other to form an alicyclic or aromatic monocyclic or polycyclic ring.

The term “substituted” in the definitions of Y₁, Y₂, and A1 to A3 in Formula 2 indicates substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, C₃-C₃₀ mixed aliphatic-aromatic cyclic groups, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof. The term “unsubstituted” in the same definition indicates having no substituent. One or more hydrogen atoms in each of the substituents are optionally replaced by deuterium atoms.

According to one embodiment of the present invention, the compound represented by Formula 2 may be a polycyclic compound represented by Formula 3 or 4:

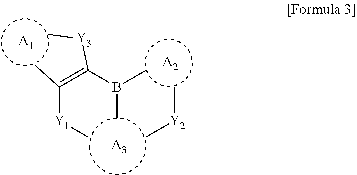

wherein Y₃ is O, S or NR18, R₁₈ is as defined for R₁₁ to R₁₇ in Formula 2, and Y₁, Y₂, and A1 to A3 are as defined in Formula 2;

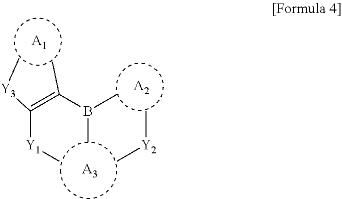

wherein Y₃ is O, S or NR18, R₁₈ is as defined for R₁₁ to R₁₇ in Formula 2, and Y₁, Y₂, and A1 to A3 are as defined in Formula 2.

According to one embodiment of the present invention, the compound represented by Formula 2 may be a polycyclic compound represented by one of Formulas 3-1, 3-2, 3-3, 4-1, 4-2, and 4-3:

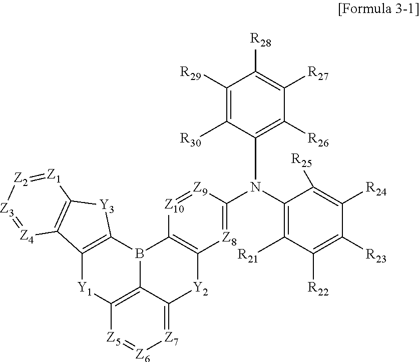

wherein Z₁ to Z₁₀ are identical to or different from each other and are each independently CR₃₁ or N, provided that when two or more of Z₁ to Z₁₀ are CR₃₁, the moieties CR₃₁ are identical to or different from each other and adjacent ones of the groups R₃₁ are optionally linked to each other to form an alicyclic or aromatic monocyclic or polycyclic ring, Y₃ is O, S or NR₁₈, R₁₈ is as defined for R₁₁ to R₁₇ in Formula 2, the groups R₃₁ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, R₂₁ to R₃₀ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl,
C₁-C₃₀ germanium, nitro, cyano, and halogen, with the proviso that one or more hydrogen atoms in each of the substituents of R₂₁ to R₃₁ are optionally replaced by deuterium atoms, adjacent ones of the substituents of R₂₁ to R₃₀ are optionally linked to each other to form an alicyclic or aromatic monocyclic or polycyclic ring, and the groups R₃₁ are optionally linked to each other to form an alicyclic or aromatic monocyclic or polycyclic ring, provided that when Z₈ and Z₉ are CR₃₁, each of R₂₁, R₂₅, R₂₆, and R₃₀ adjacent to Z₈ and Z₉ is optionally bonded to R₃₁ to form an alicyclic or aromatic monocyclic or polycyclic ring, Y₁ and Y₂ are identical to or different from each other and are each independently as defined in Formula 2 or are each independently a linker represented by Structural Formula A:

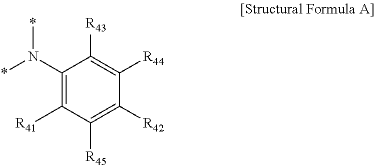

wherein R₄₁ to R₄₅ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in each of the substituents of R₄₁ to R₄₅ are optionally replaced by deuterium atoms and each of R₄₁ to R₄₅ is optionally linked to one or more adjacent substituents to form an alicyclic or aromatic monocyclic or polycyclic ring;

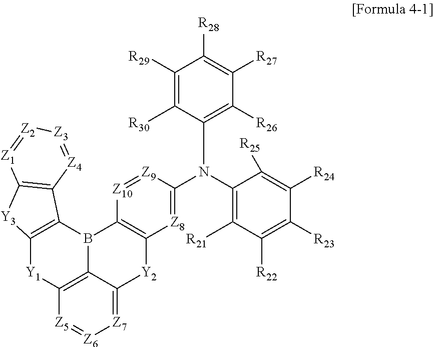

wherein Z₁ to Z₁₀, Y₁ to Y₃, and R₂₁ to R₃₀ are as defined in Formula 3-1;

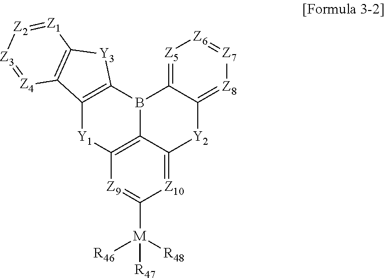

wherein Z₁ to Zio are identical to or different from each other and are each independently CR₃₁ or N, provided that when two or more of Z₁ to Zio are CR₃₁, the moieties CR₃₁ are identical to or different from each other and when two or more of Z₁ to Z₈ are CR₃₁, adjacent ones of the groups R₃₁ are optionally linked to each other to form an alicyclic or aromatic monocyclic or polycyclic ring, Y₃ is O, S or NR18, R₁₈ is as defined for R₁₁ to R₁₇ in Formula 2, M is Si or Ge, the groups R₃₁ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, R₄₆ to R₄₈ are identical to or different from each other and are each independently selected from hydrogen, deuterium, substituted or unsubstituted C₁-C₂₄ alkyl, substituted or unsubstituted C₂-C₂₄ alkynyl, substituted or unsubstituted C₂-C₂₄ alkenyl, substituted or unsubstituted C₆-C₃₀ aryl, substituted or unsubstituted C₃-C₂₄ cycloalkyl, substituted or unsubstituted C₂-C₂₄ heterocycloalkyl, substituted or unsubstituted C₂-C₃₀ heteroaryl, substituted or unsubstituted C₃-C₃₀ mixed aliphatic-aromatic cyclic groups, substituted or unsubstituted C₁-C₂₄ alkoxy, substituted or unsubstituted C₆-C₂₄ aryloxy, substituted or unsubstituted C₁-C₂₄ alkylthioxy, substituted or unsubstituted C₅-C₂₄ arylthioxy, substituted or unsubstituted C₁-C₃₀ amine, substituted or unsubstituted C₁-C₃₀ silyl, substituted or unsubstituted C₁-C₃₀ germanium, nitro, cyano, and halogen, with the proviso that one or more hydrogen atoms in each of the substituents of R₃₁ and R₄₆ to R₄₈ are optionally replaced by deuterium atoms and the substituents of R₄₆ to R₄₈ are optionally linked to each other to form an alicyclic or aromatic monocyclic or polycyclic ring;

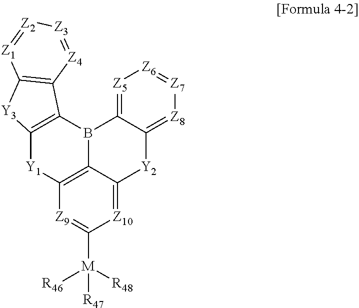

wherein Z₁ to Z₁₀, Y₁ to Y₃, M, and R₄₆ to R₄₈ are as defined in Formula 3-2;

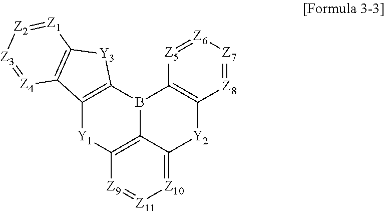

wherein Z₁ to Z₁₁ are identical to or different from each other and are each independently CR₃₁ or N, provided that when two or more of Z₁ to Z₁₁ are CR₃₁, the moieties CR₃₁ are identical to or different from each other and adjacent ones of the groups R₃₁ are optionally linked to each other to form an alicyclic or aromatic monocyclic or polycyclic ring, Y₃ is O, S or NR18, R₁₈ is as defined for R₁ to R₁₇ in Formula 2, the groups R₃₁ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, with the proviso that one or more hydrogen atoms in each of the substituents of the groups R₃₁ are optionally replaced by deuterium atoms, Y₁ and Y₂ are identical to or different from each other and are each independently as defined in Formula 2 or are each independently a linker represented by Structural Formula B:

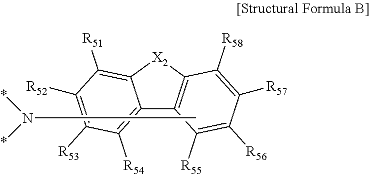

wherein X₂ is O or S, R₅₁ to R₅₈ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in each of the substituents of R₅₁ to R₅₈ are optionally replaced by deuterium atoms, one of R₅₁ to R₅₈ forms a single bond with the nitrogen atom, and each of the others is optionally linked to one or more adjacent substituents to form an alicyclic or aromatic monocyclic or polycyclic ring; and

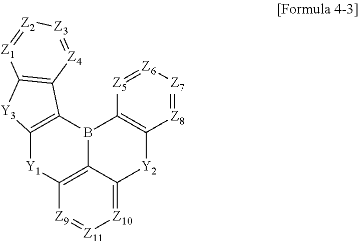

wherein Z₁ to Z₁₁ and Y₁ to Y₃ are as defined in Formula 3-3.

The term “substituted” in the definitions of Z₁ to Z₁₀, Y₁ to Y₃, and R₂₁ to R₃₀ in Formula 3-1, Z₁ to Z₁₀, Y₁ to Y₃, and R₄₆ to R₄₈ in Formula 3-2, Z₁ to Z₁₁ and Y₁ to Y₃ in Formula 3-3, Z₁ to Zio, Y₁ to Y₃, and R₂₁ to R₃₀ in Formula 4-1, Z₁ to Z₁₀, Y₁ to Y₃, and R₄₆ to R₄₈ in Formula 4-2, and Z₁ to Z₁₁ and Y₁ to Y₃ in Formula 4-3 indicates substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, C₃-C₃₀ mixed aliphatic-aromatic cyclic groups, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof. The term “unsubstituted” in the same definition indicates having no substituent. One or more hydrogen atoms in each of the substituents are optionally replaced by deuterium atoms.

The content of the dopant in the light emitting layer of the organic light emitting device according to the present invention is typically in the range of about 0.01 to about 20 parts by weight, based on about 100 parts by weight of the host but is not limited to this range.

The light emitting layer may further include one or more dopants other than the polycyclic compound represented by Formula 2 and one or more hosts other than the compound represented by Formula 1. In this case, the hosts and the dopants may be mixed or stacked in the light emitting layer.

In the “substituted or unsubstituted C₁-C₃₀ alkyl”, “substituted or unsubstituted C₆-C₅₀ aryl”, etc., the number of carbon atoms in the alkyl or aryl group indicates the number of carbon atoms constituting the unsubstituted alkyl or aryl moiety without considering the number of carbon atoms in the substituent(s). For example, a phenyl group substituted with a butyl group at the para-position corresponds to a C₆ aryl group substituted with a C₄ butyl group.

As used herein, the expression “optionally linked to each other or an adjacent group to form a ring” means that the corresponding adjacent substituents are bonded to each other or each of the corresponding substituents is bonded to an adjacent group to form a substituted or unsubstituted alicyclic or aromatic ring. The term “adjacent group” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other. Optionally, the paired substituents each lose one hydrogen radical and are linked to each other to form a ring. The carbon atoms in the resulting alicyclic, aromatic mono- or polycyclic ring may be replaced by one or more heteroatoms such as N, NR (wherein R is as defined for R₁₁ to R₆), O, S, Si, and Ge.

In the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In the present invention, specific examples of the arylalkyl groups include, but are not limited to, phenylmethyl (benzyl), phenylethyl, phenylpropyl, naphthylmethyl, and naphthylethyl.

In the present invention, specific examples of the alkylaryl groups include, but are not limited to, tolyl, xylenyl, dimethylnaphthyl, t-butylphenyl, t-butylnaphthyl, and t-butylphenanthryl.

The alkenyl group is intended to include straight and branched ones and may be optionally substituted with one or more other substituents. The alkenyl group may be specifically a vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl) vinyl-1-yl, stilbenyl or styrenyl group but is not limited thereto.

The alkynyl group is intended to include straight and branched ones and may be optionally substituted with one or more other substituents. The alkynyl group may be, for example, ethynyl or 2-propynyl but is not limited thereto.

The cycloalkenyl group is a non-aromatic cyclic unsaturated hydrocarbon group having one or more carbon-carbon double bonds. The cycloalkenyl group may be, for example, cyclopropenyl, cyclobutenyl, cyclopentenyl, cyclohexenyl, cycloheptenyl, 1,3-cyclohexadienyl, 1,4-cyclohexadienyl, 2,4-cycloheptadienyl or 1,5-cyclooctadienyl but is not limited thereto.

The aromatic hydrocarbon rings or aryl groups may be monocyclic or polycyclic ones. As used herein, the term “polycyclic” means that the aromatic hydrocarbon ring may be directly attached or fused to one or more other cyclic groups. The other cyclic groups may be aromatic hydrocarbon rings and other examples thereof include aliphatic heterocyclic rings, aliphatic hydrocarbon rings, and aromatic heterocyclic rings. Examples of the monocyclic aryl groups include, but are not limited to, phenyl, biphenyl, and terphenyl. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups but the scope of the present invention is not limited thereto.

The aromatic heterocyclic rings or heteroaryl groups refer to aromatic groups containing one or more heteroatoms. Examples of the aromatic heterocyclic rings or heteroaryl groups include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, and phenothiazinyl groups.

The aliphatic hydrocarbon rings or cycloalkyl groups refer to non-aromatic rings consisting only of carbon and hydrogen atoms. The aliphatic hydrocarbon ring is intended to include monocyclic and polycyclic ones and may be optionally substituted with one or more other substituents. As used herein, the term “polycyclic” means that the aliphatic hydrocarbon ring may be directly attached or fused to one or more other cyclic groups. The other cyclic groups may be aliphatic hydrocarbon rings and other examples thereof include aliphatic heterocyclic rings, aromatic hydrocarbon rings, and aromatic heterocyclic rings. Specific examples of the aliphatic hydrocarbon rings include, but are not limited to, cycloalkyl groups such as cyclopropyl, cyclobutyl, cyclopentyl, adamantyl, bicycloheptanyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, and cyclooctyl, cycloalkanes such as cyclohexane and cyclopentane, and cycloalkenes such as cyclohexene and cyclobutene.

The aliphatic heterocyclic rings or heterocycloalkyl groups refer to aliphatic rings containing one or more heteroatoms such as O, S, Se, N, and Si. The aliphatic heterocyclic ring is intended to include monocyclic or polycyclic ones and may be optionally substituted with one or more other substituents. As used herein, the term “polycyclic” means that the aliphatic heterocyclic ring such as heterocycloalkyl or heterocycloalkane may be directly attached or fused to one or more other cyclic groups. The other cyclic groups may be aliphatic heterocyclic rings and other examples thereof include aliphatic hydrocarbon rings, aromatic hydrocarbon rings, and aromatic heterocyclic rings.

The mixed aliphatic-aromatic cyclic group refers to a ring in which an aliphatic ring and an aromatic ring are linked and fused together and which are overall non-aromatic. More specifically, the mixed aliphatic-aromatic cyclic group may be a cycloalkyl group fused with an aromatic hydrocarbon ring, a cycloalkyl group fused with an aromatic heterocyclic ring, a heterocycloalkyl group fused with an aromatic hydrocarbon ring, an aryl group fused with an aliphatic hydrocarbon ring, a heteroaryl group fused with an aliphatic hydrocarbon ring, an aryl group fused with an aliphatic heterocyclic ring or a heteroaryl group fused with an aliphatic heterocyclic ring. Specific examples of such mixed aliphatic-aromatic cyclic groups include tetrahydronaphthyl, tetrahydrobenzocycloheptene, tetrahydrophenanthrene, tetrahydroanthracenyl, octahydrotriphenylene, tetrahydrobenzothiophene, tetrahydrobenzofuranyl, tetrahydrocarbazole, and tetrahydroquinoline. The mixed aliphatic-aromatic cyclic group may be interrupted by at least one heteroatom other than carbon. The heteroatom may be, for example, N, O, S, Si, Ge or P.

The alkoxy group may be specifically a methoxy, ethoxy, propoxy, isobutyloxy, sec-butyloxy, pentyloxy, iso-amyloxy or hexyloxy group but is not limited thereto.

The silyl group is intended to include —SiH₃, alkylsilyl, arylsilyl, alkylarylsilyl, arylheteroarylsilyl, and heteroarylsilyl. The arylsilyl refers to a silyl group obtained by substituting one, two or three of the hydrogen atoms in —SiH₃ with aryl groups. The alkylsilyl refers to a silyl group obtained by substituting one, two or three of the hydrogen atoms in —SiH₃ with alkyl groups. The alkylarylsilyl refers to a silyl group obtained by substituting one of the hydrogen atoms in —SiH₃ with an alkyl group and the other two hydrogen atoms with aryl groups or substituting two of the hydrogen atoms in —SiH₃ with alkyl groups and the remaining hydrogen atom with an aryl group. The arylheteroarylsilyl refers to a silyl group obtained by substituting one of the hydrogen atoms in —SiH₃ with an aryl group and the other two hydrogen atoms with heteroaryl groups or substituting two of the hydrogen atoms in —SiH₃ with aryl groups and the remaining hydrogen atom with a heteroaryl group. The heteroarylsilyl refers to a silyl group obtained by substituting one, two or three of the hydrogen atoms in —SiH₃ with heteroaryl groups. The arylsilyl group may be, for example, substituted or unsubstituted monoarylsilyl, substituted or unsubstituted diarylsilyl, or substituted or unsubstituted triarylsilyl. The same applies to the alkylsilyl and heteroarylsilyl groups.

Each of the aryl groups in the arylsilyl, heteroarylsilyl, and arylheteroarylsilyl groups may be a monocyclic or polycyclic one. Each of the heteroaryl groups in the arylsilyl, heteroarylsilyl, and arylheteroarylsilyl groups may be a monocyclic or polycyclic one.

Specific examples of the silyl groups include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more of the hydrogen atoms in each of the silyl groups may be substituted with the substituents mentioned in the aryl groups.

The amine group is intended to include —NH₂, alkylamine, arylamine, arylheteroarylamine, and heteroarylamine. The arylamine refers to an amine group obtained by substituting one or two of the hydrogen atoms in —NH₂ with aryl groups. The alkylamine refers to an amine group obtained by substituting one or two of the hydrogen atoms in —NH₂ with alkyl groups. The alkylarylamine refers to an amine group obtained by substituting one of the hydrogen atoms in —NH₂ with an alkyl group and the other hydrogen atom with an aryl group. The arylheteroarylamine refers to an amine group obtained by substituting one of the hydrogen atoms in —NH₂ with an aryl group and the other hydrogen atom with a heteroaryl group. The heteroarylamine refers to an amine group obtained by substituting one or two of the hydrogen atoms in —NH₂ with heteroaryl groups. The arylamine may be, for example, substituted or unsubstituted monoarylamine, substituted or unsubstituted diarylamine, or substituted or unsubstituted triarylamine. The same applies to the alkylamine and heteroarylamine groups.

Each of the aryl groups in the arylamine, heteroarylamine, and arylheteroarylamine groups may be a monocyclic or polycyclic one. Each of the heteroaryl groups in the arylamine, heteroarylamine, and arylheteroarylamine groups may be a monocyclic or polycyclic one.

The germanium group is intended to include —GeH₃, alkylgermanium, arylgermanium, heteroarylgermanium, alkylarylgermanium, alkylheteroarylgermanium, and arylheteroarylgermanium. The definitions of the substituents in the germanium groups follow those described for the silyl groups, except that the silicon (Si) atom in each silyl group is changed to a germanium (Ge) atom.

Specific examples of the germanium groups include trimethylgermane, triethylgermane, triphenylgermane, trimethoxygermane, dimethoxyphenylgermane, diphenylmethylgermane, diphenylvinylgermane, methylcyclobutylgermane, and dimethylfurylgermane. One or more of the hydrogen atoms in each of the germanium groups may be substituted with the substituents mentioned in the aryl groups.

The cycloalkyl, aryl, and heteroaryl groups in the cycloalkyloxy, aryloxy, heteroaryloxy, cycloalkylthioxy, arylthioxy, and heteroarylthioxy groups are the same as those exemplified above. Specific examples of the aryloxy groups include, but are not limited to, phenoxy, p-tolyloxy, m-tolyloxy, 3,5-dimethylphenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, and 9-phenanthryloxy groups. Specific examples of the arylthioxy groups include, but are not limited to, phenylthioxy, 2-methylphenylthioxy, and 4-tert-butylphenylthioxy groups.

The halogen group may be, for example, fluorine, chlorine, bromine or iodine.

According to one embodiment of the present invention, the anthracene derivative represented by Formula 1 may be selected from the following compounds:

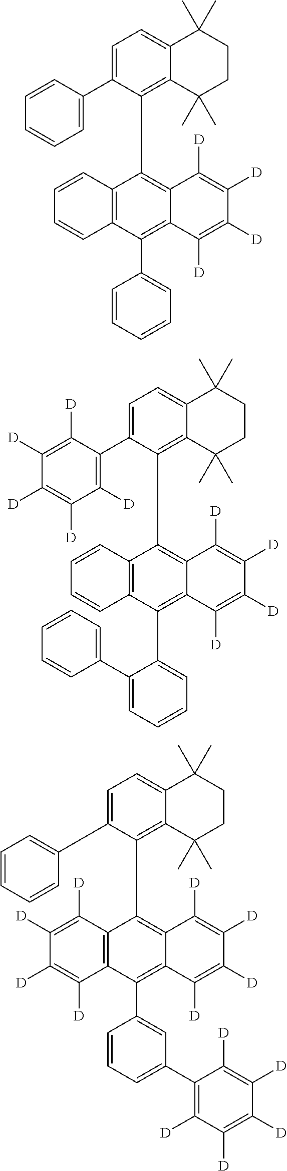

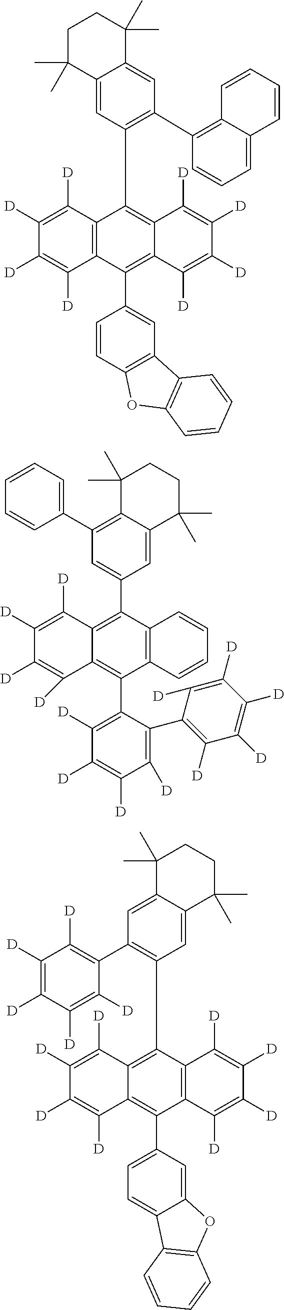

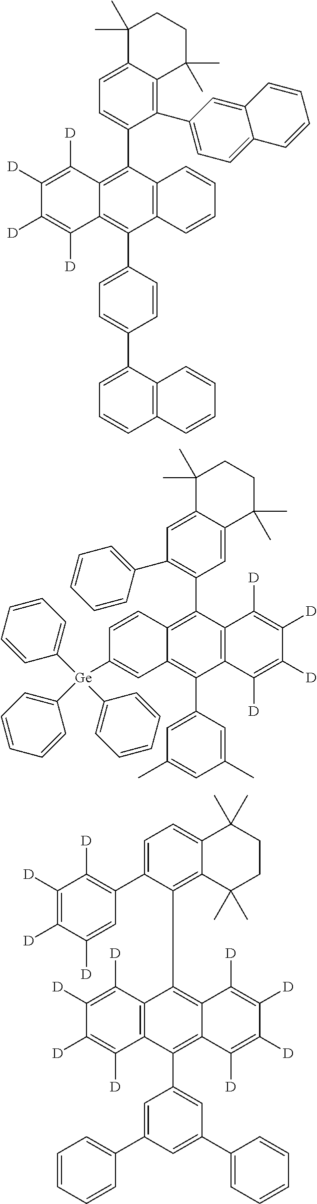

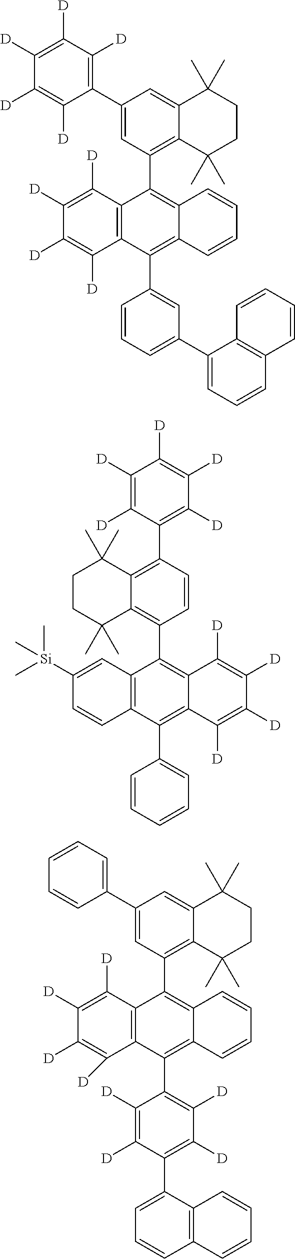 [SMILES: [2H]c1c([2H])c([2H])c(-c2cc(-c3c4ccccc4c(-c4cccc(-c5cccc6ccccc56)c4)c4c([2H])c([2H])c([2H])c([2H])c34)c3c(c2)C(C)(C)CCC3(C)C)c([2H])c1[2H] ; [2H]c1c([2H])c([2H])c(-c2ccc(-c3c4cc([Si](C)(C)C)ccc4c(-c4ccccc4)c4c([2H])c([2H])c([2H])c([2H])c34)c3c2C(C)(C)CCC3(C)C)c([2H])c1[2H] ; [2H]c1c([2H])c(-c2c3ccccc3c(-c3cc(-c4ccccc4)cc4c3C(C)(C)CCC4(C)C)c3c([2H])c([2H])c([2H])c([2H])c23)c([2H])c([2H])c1-c1cccc2ccccc12]

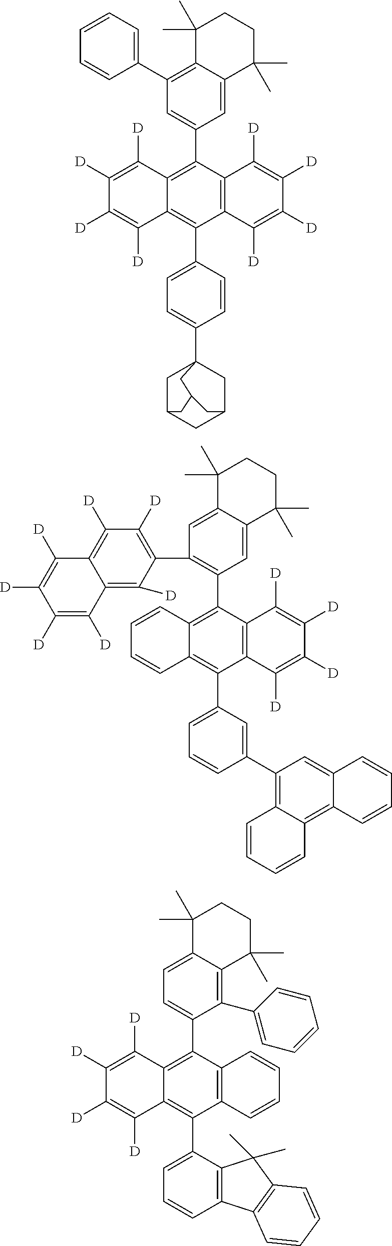

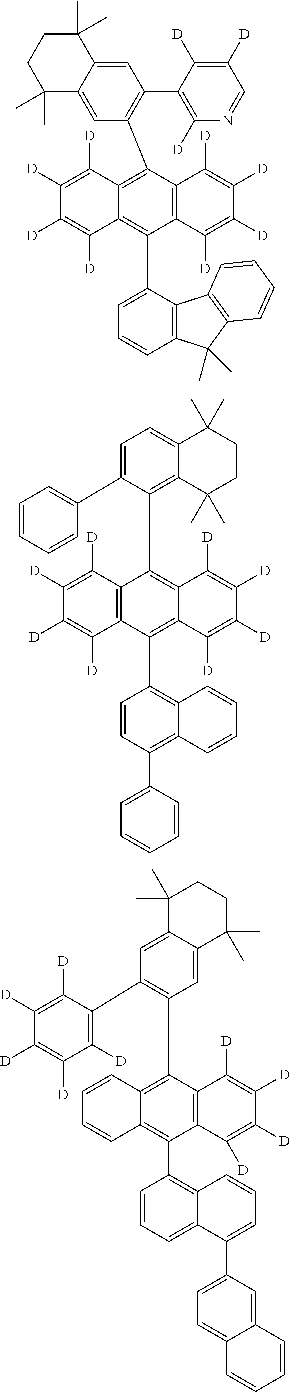

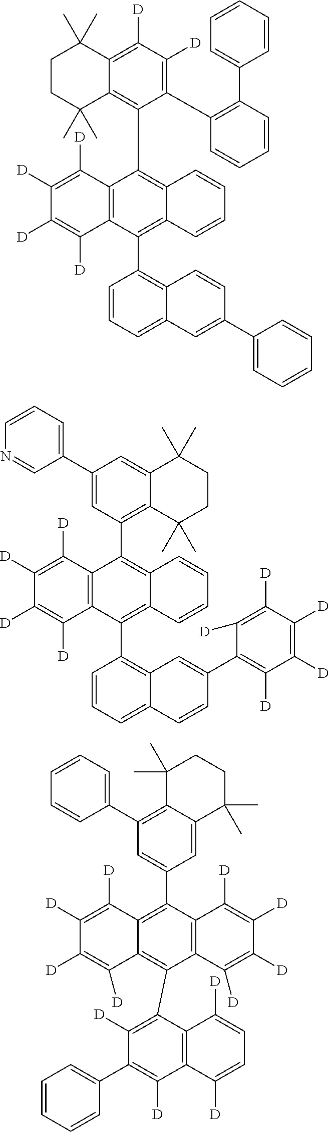

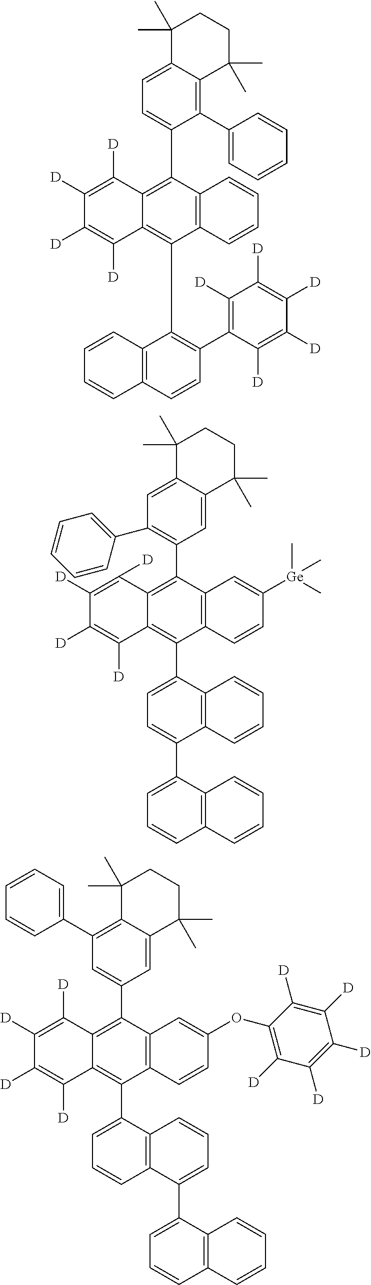

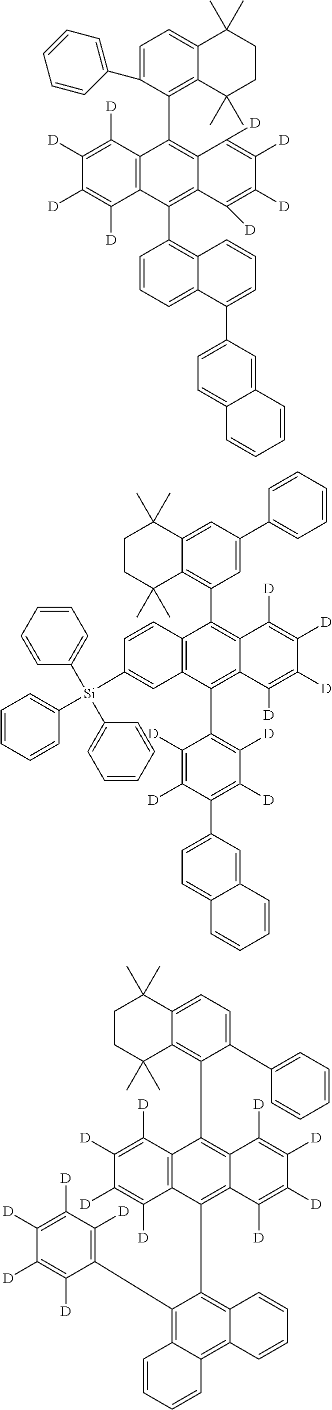

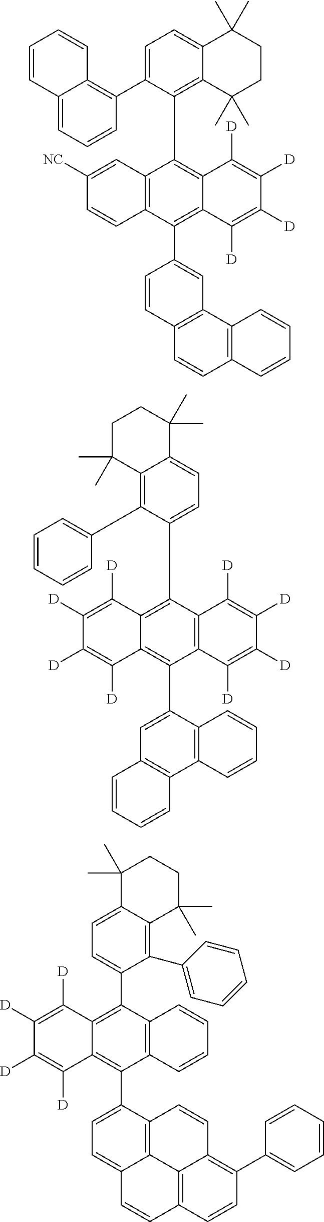

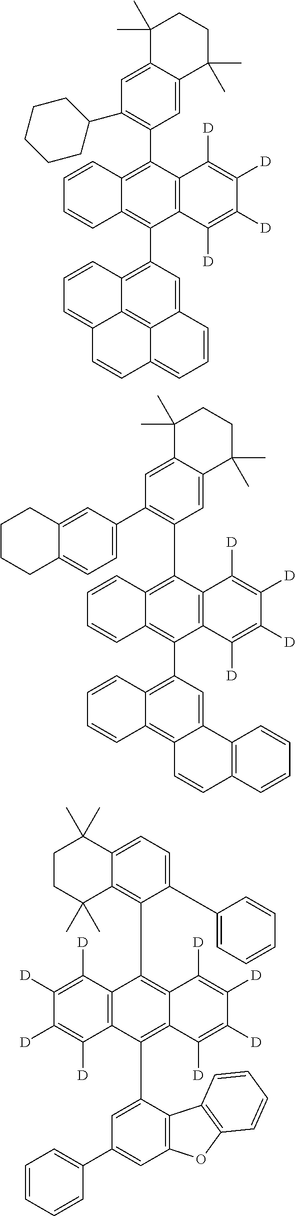

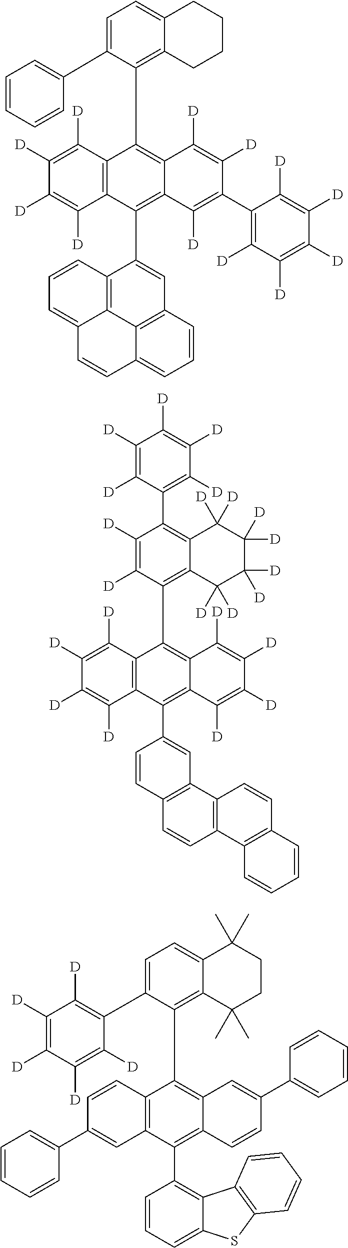

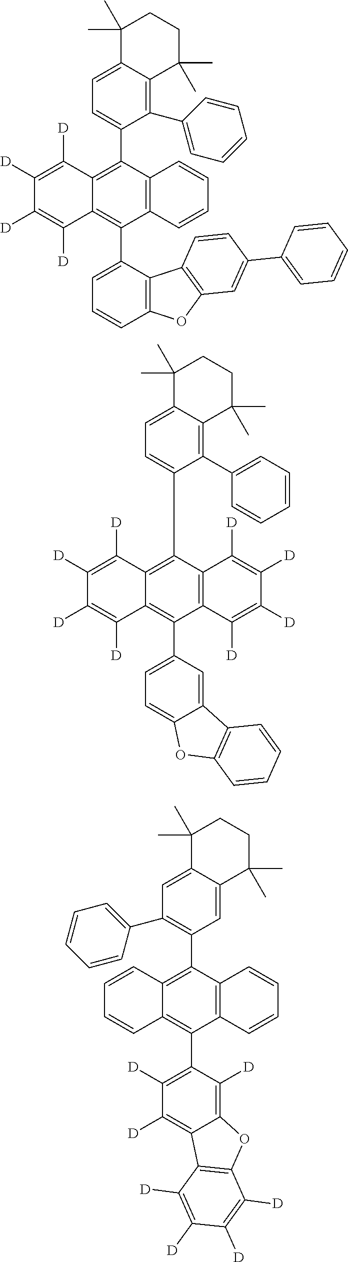

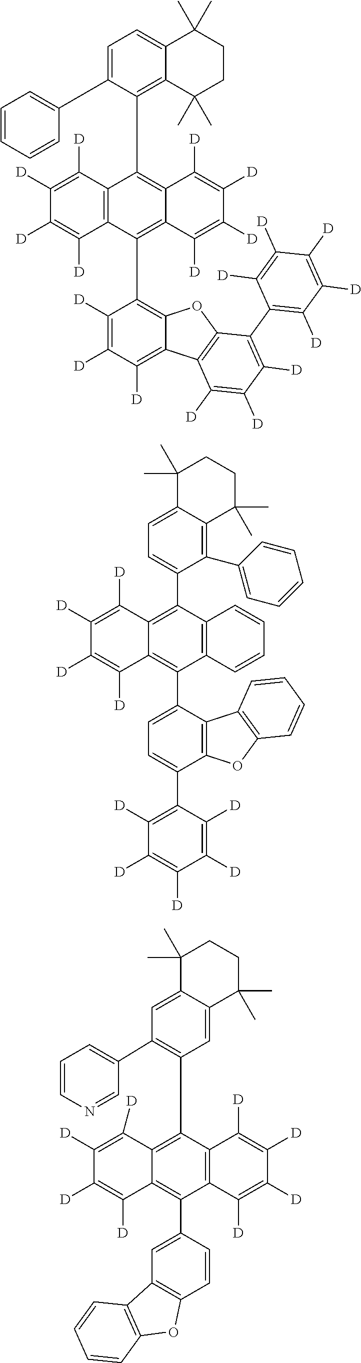

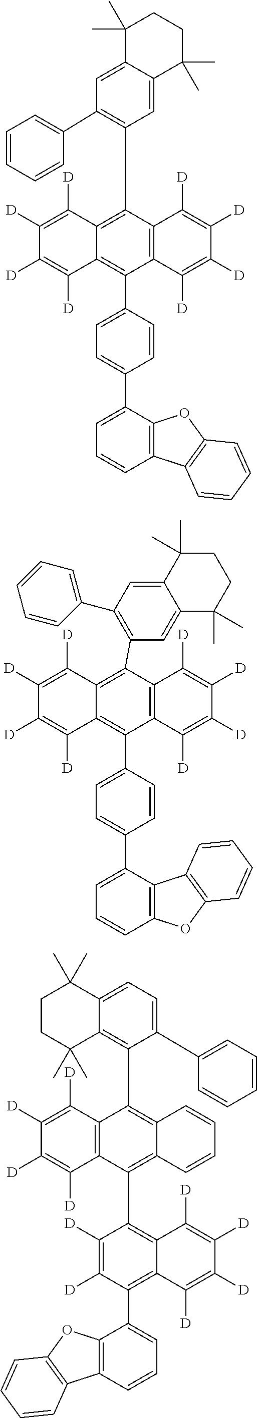

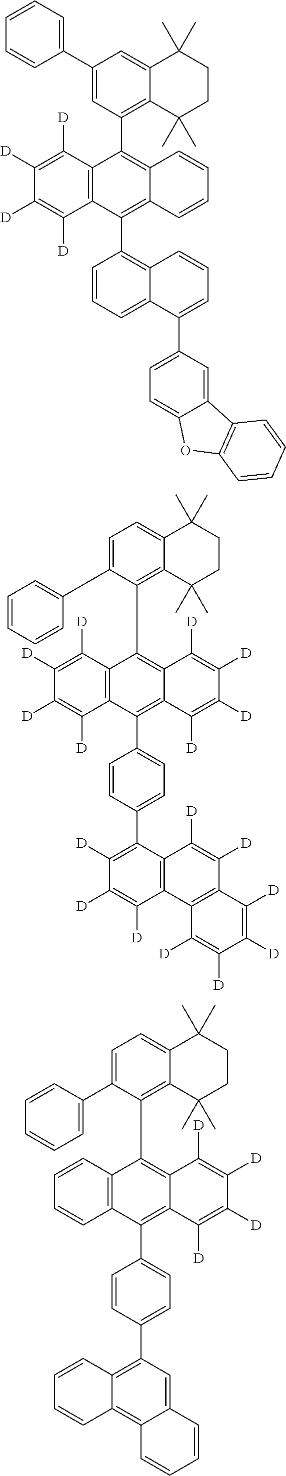

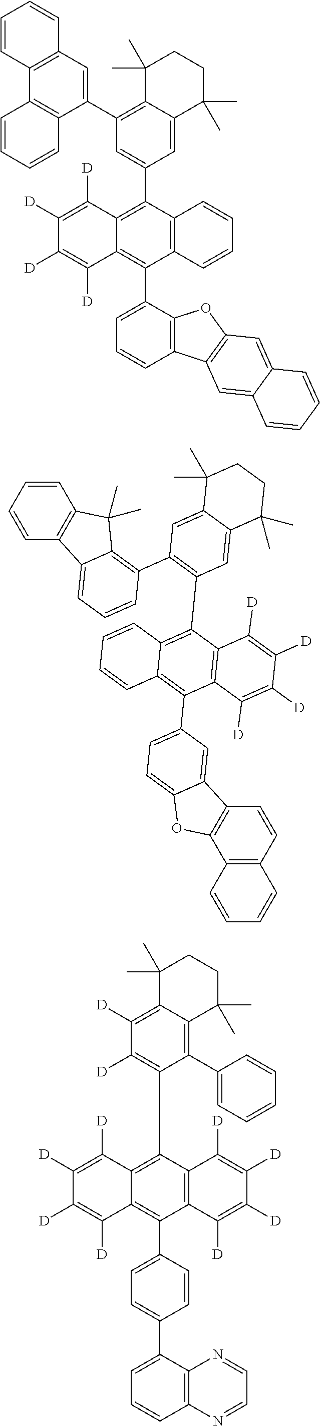 [SMILES: [2H]c1c([2H])c([2H])c2c(-c3cccc4c3oc3cc5ccccc5cc34)c3ccccc3c(-c3cc(-c4cc5ccccc5c5ccccc45)c4c(c3)C(C)(C)CCC4(C)C)c2c1[2H] ; [2H]c1c([2H])c([2H])c2c(-c3cc4c(cc3-c3cccc5c3C(C)(C)c3ccccc3-5)C(C)(C)CCC4(C)C)c3ccccc3c(-c3ccc4oc5c6ccccc6ccc5c4c3)c2c1[2H] ; [2H]c1c([2H])c2c(c(-c3ccccc3)c1-c1c3c([2H])c([2H])c([2H])c([2H])c3c(-c3ccc(-c4cccc5nccnc45)cc3)c3c([2H])c([2H])c([2H])c([2H])c13)C(C)(C)CCC2(C)C]

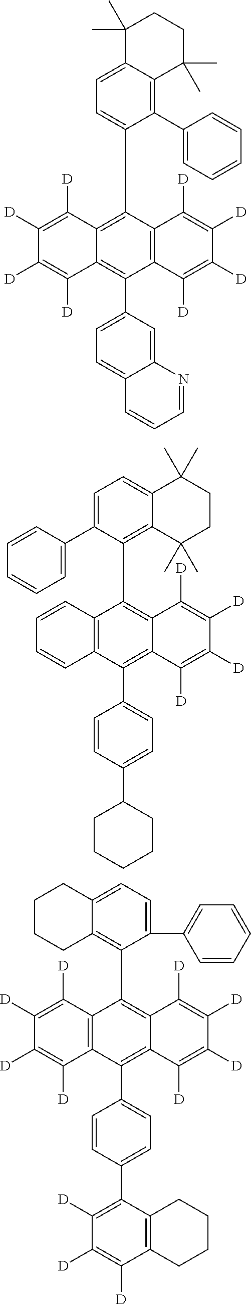

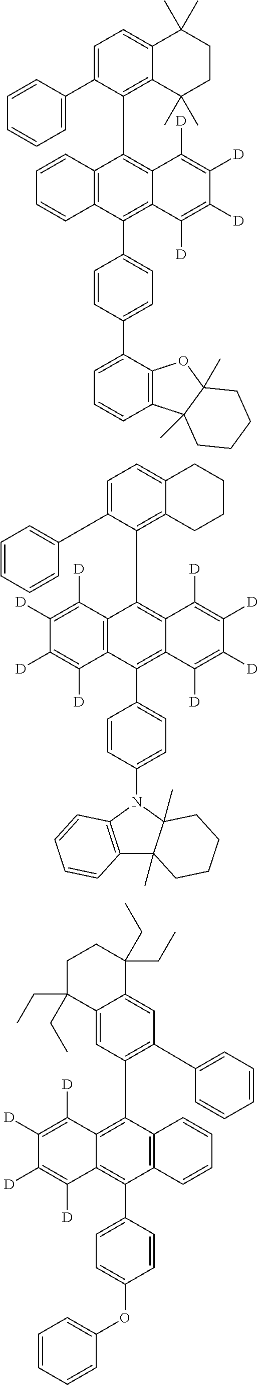

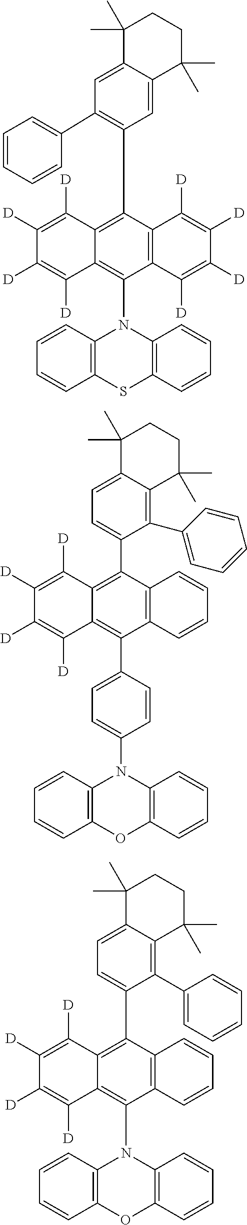

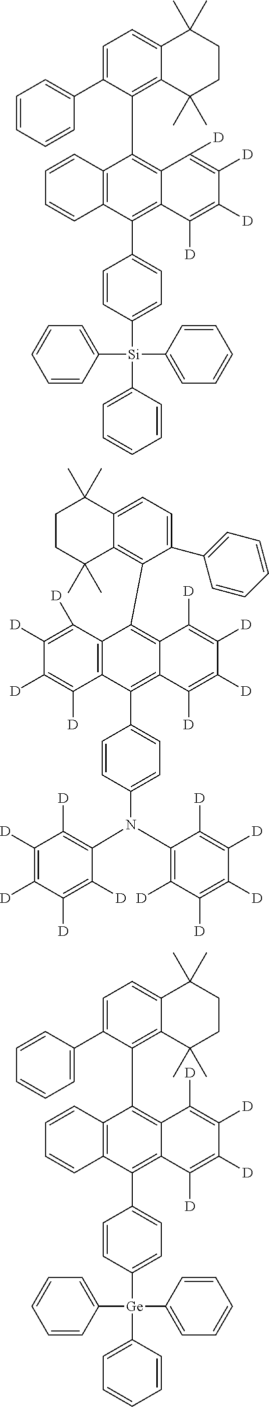

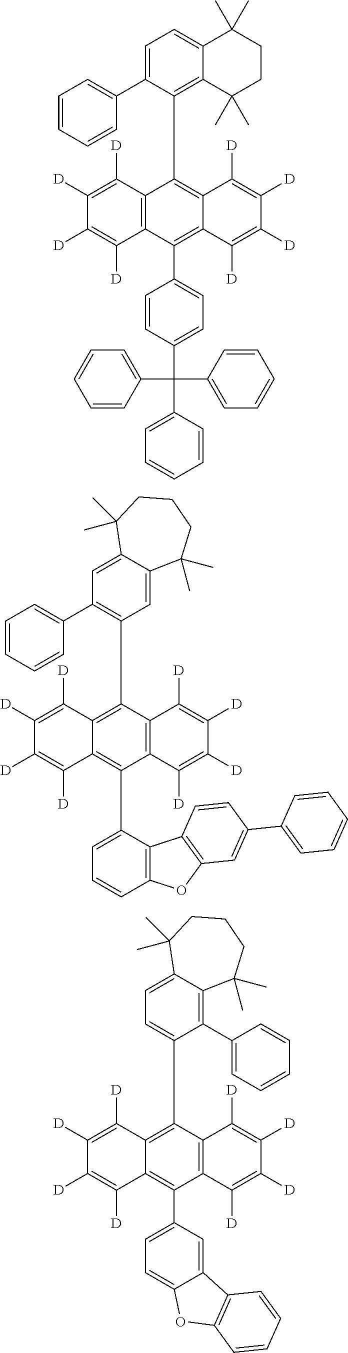

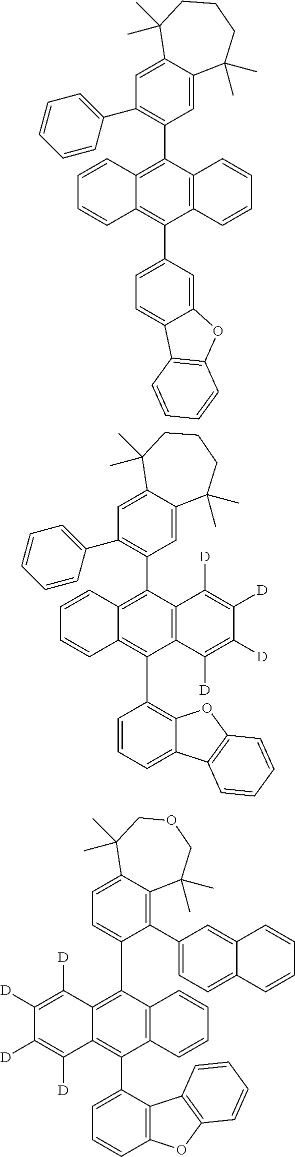

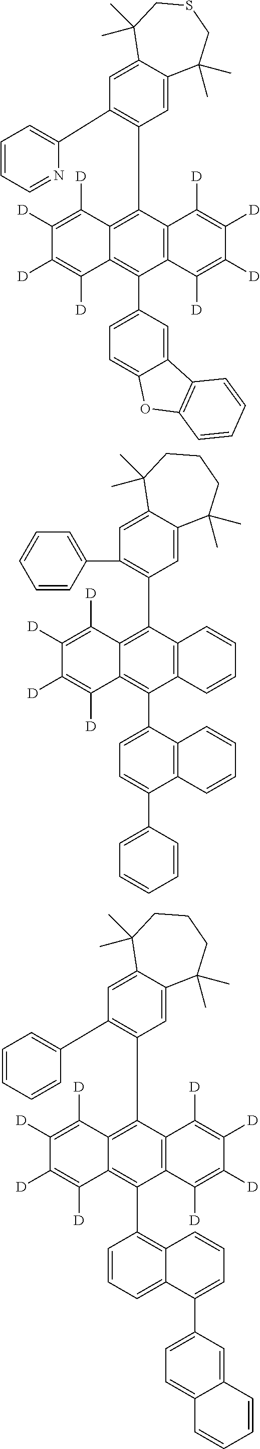

However, these compounds are not intended to limit the scope of Formula 1.

According to one embodiment of the present invention, the polycyclic compound represented by Formula 2 may be selected from the following compounds:

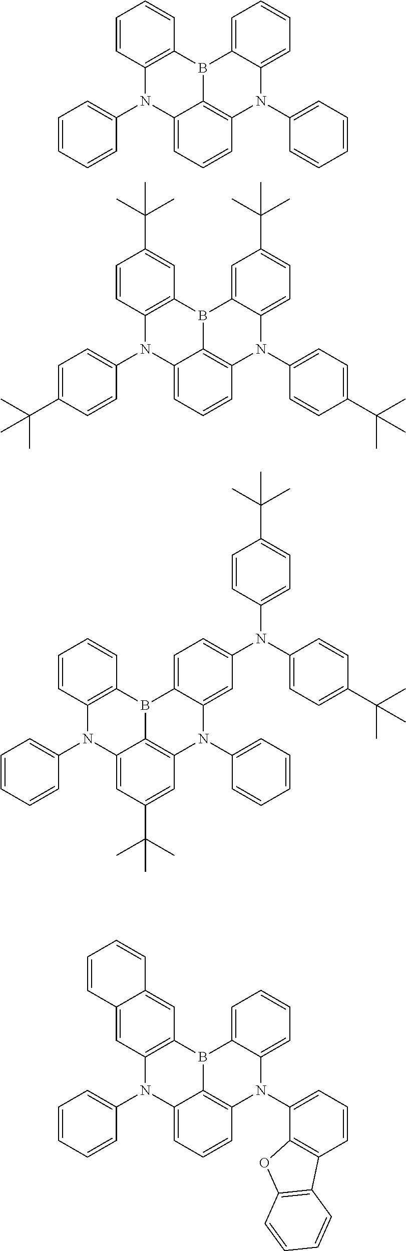

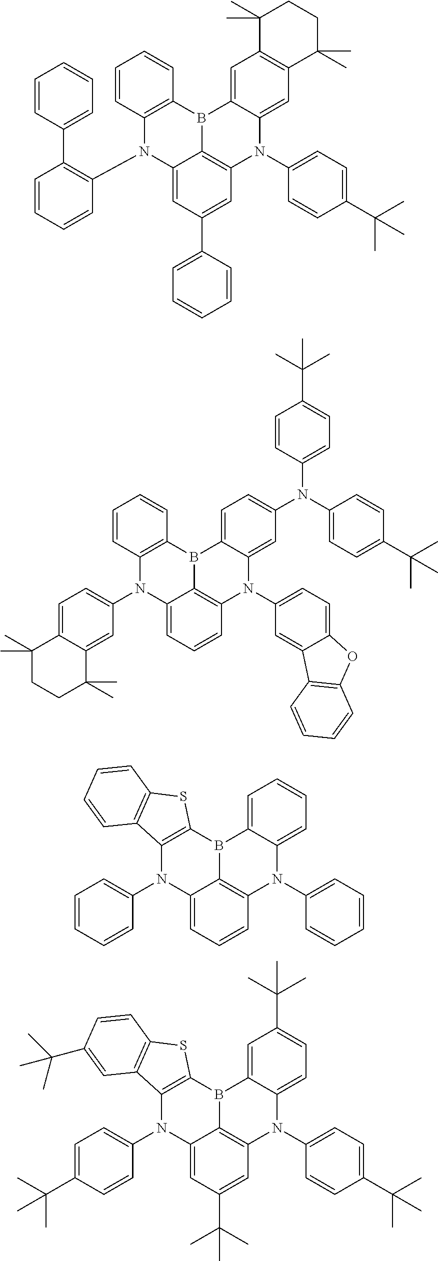

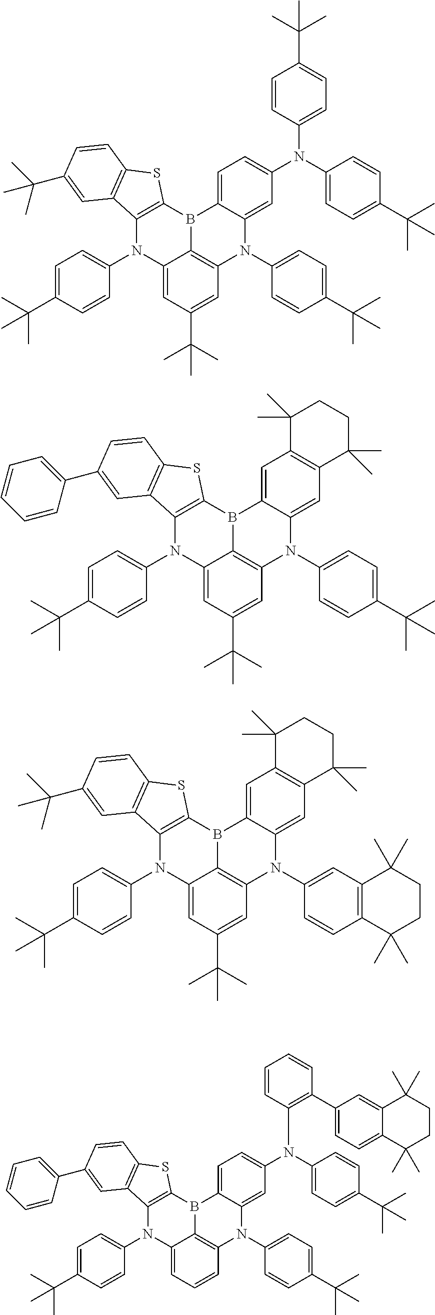

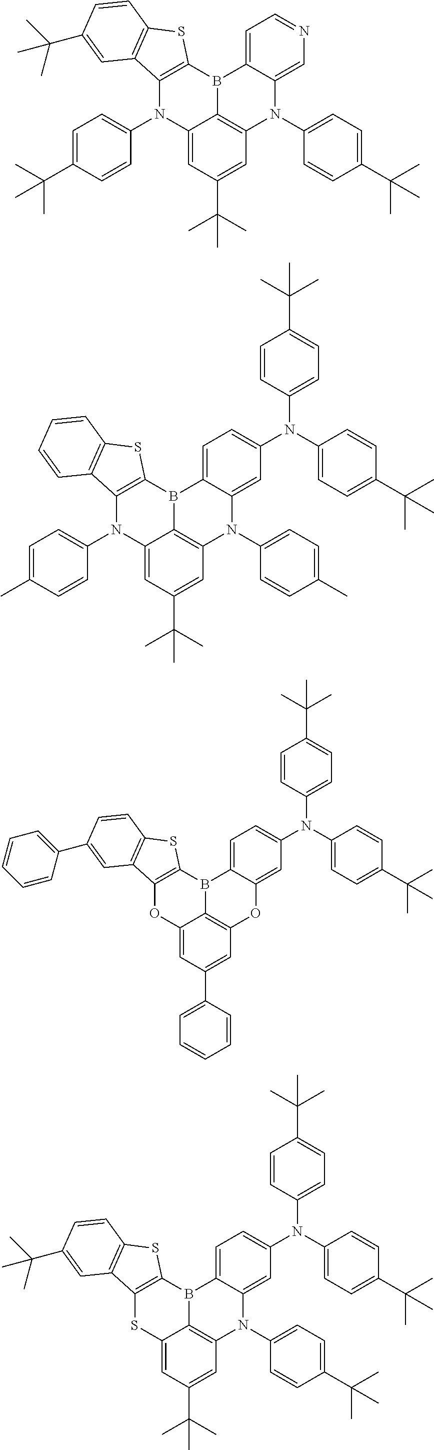

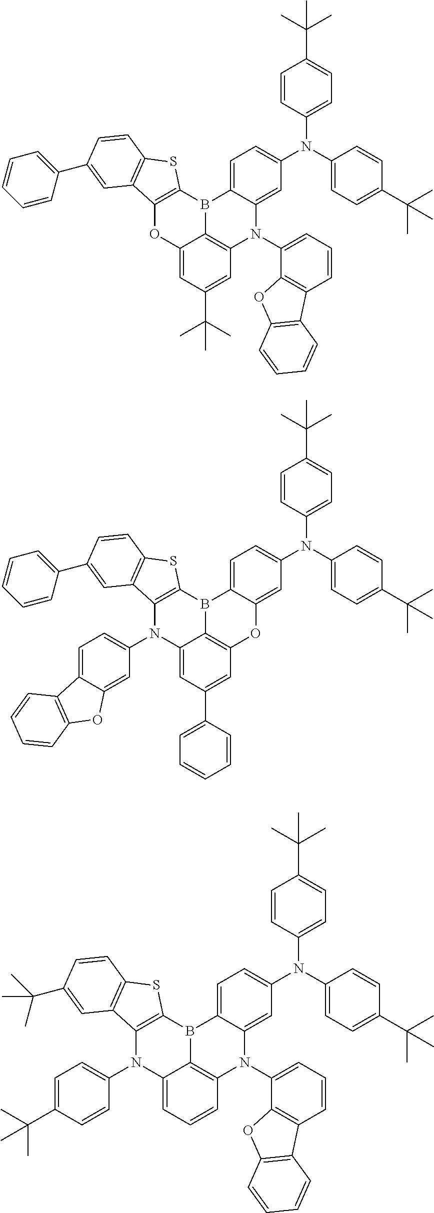

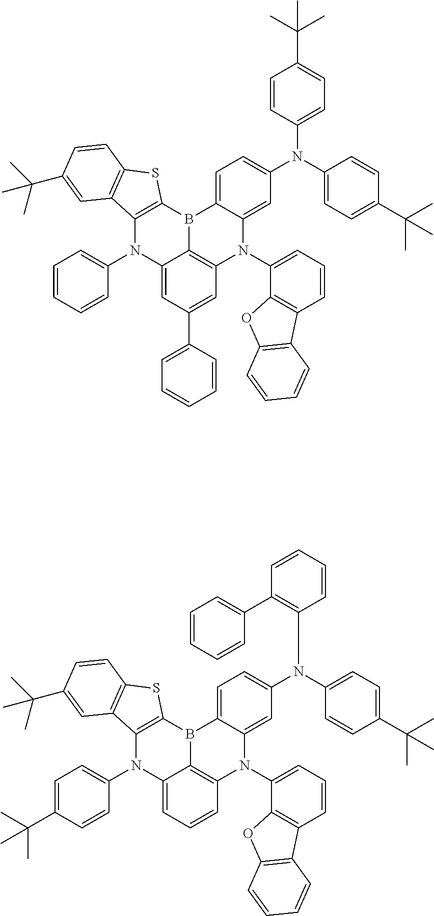

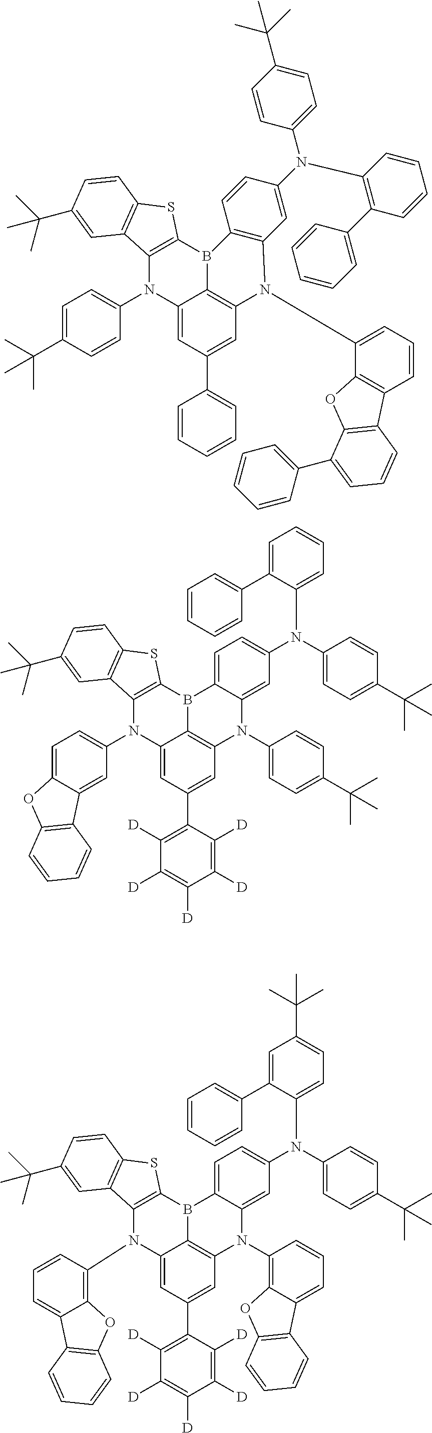

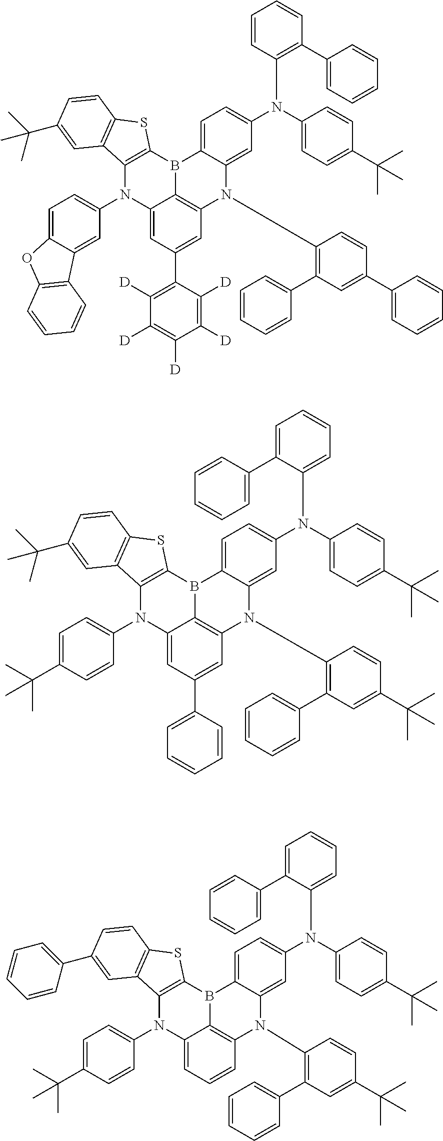 [SMILES: [2H]c1c([2H])c([2H])c(-c2cc3c4c(c2)N(c2ccc5oc6ccccc6c5c2)c2c(sc5ccc(C(C)(C)C)cc25)B4c2ccc(N(c4ccc(C(C)(C)C)cc4)c4ccccc4-c4ccccc4)cc2N3c2ccc(-c3ccccc3)cc2-c2ccccc2)c([2H])c1[2H] ; CC(C)(C)c1ccc(N(c2ccc3c(c2)N(c2ccc(C(C)(C)C)cc2-c2ccccc2)c2cc(-c4ccccc4)cc4c2B3c2sc3ccc(C(C)(C)C)cc3c2N4c2ccc(C(C)(C)C)cc2)c2ccccc2-c2ccccc2)cc1 ; CC(C)(C)c1ccc(N(c2ccc3c(c2)N(c2ccc(C(C)(C)C)cc2-c2ccccc2)c2cccc4c2B3c2sc3ccc(-c5ccccc5)cc3c2N4c2ccc(C(C)(C)C)cc2)c2ccccc2-c2ccccc2)cc1]

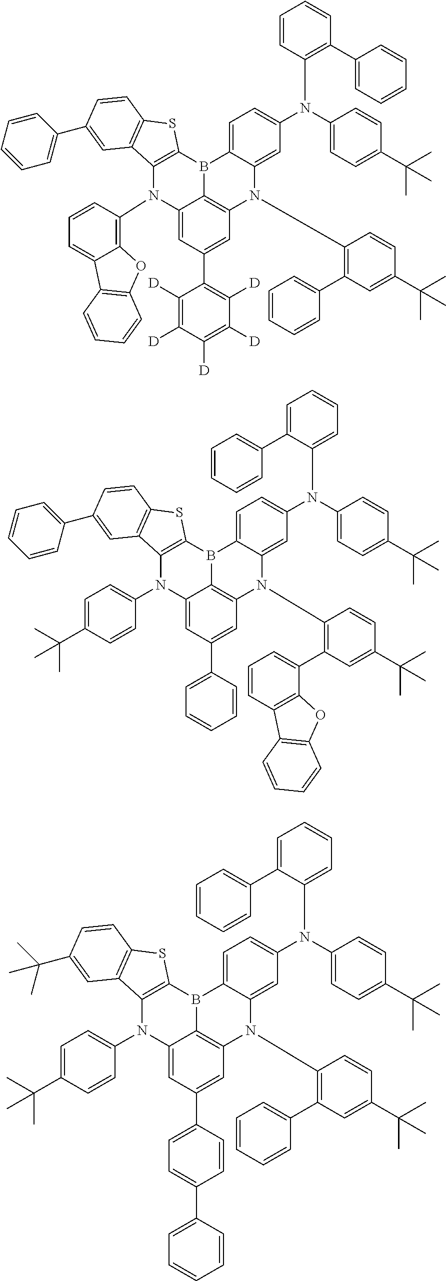

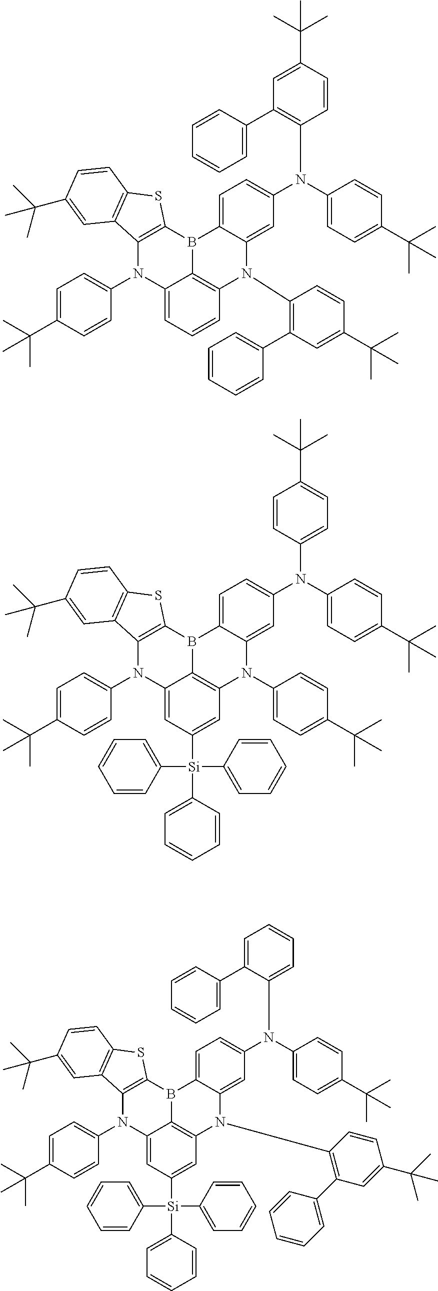

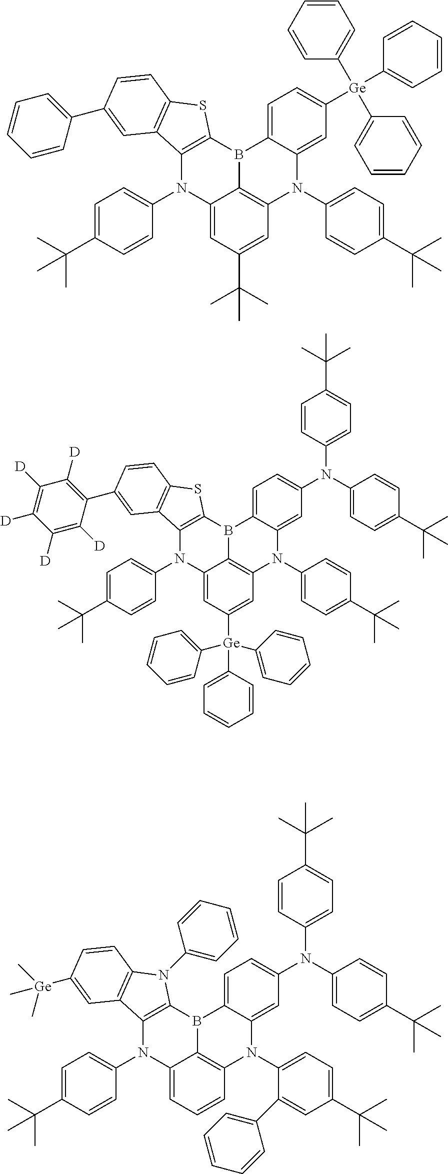

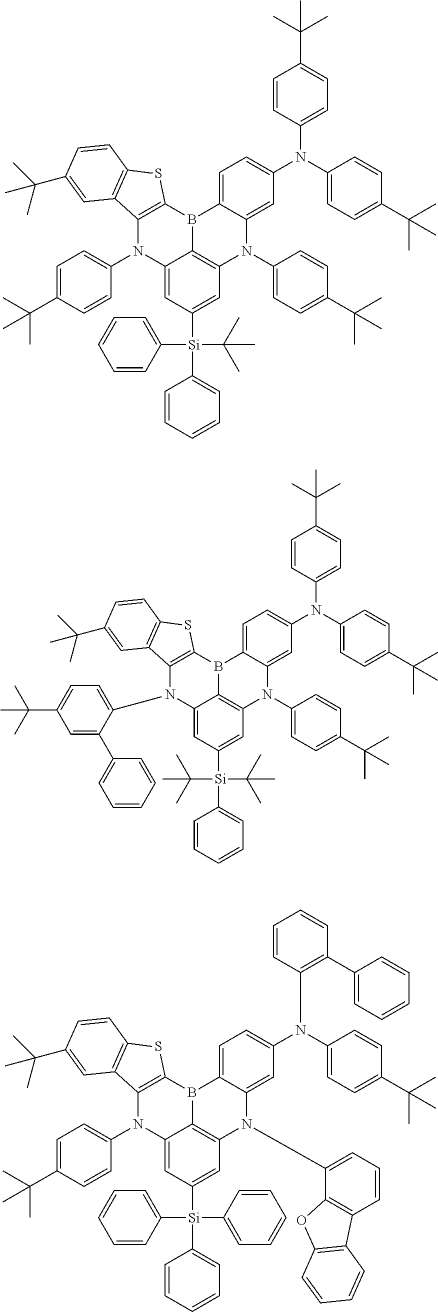

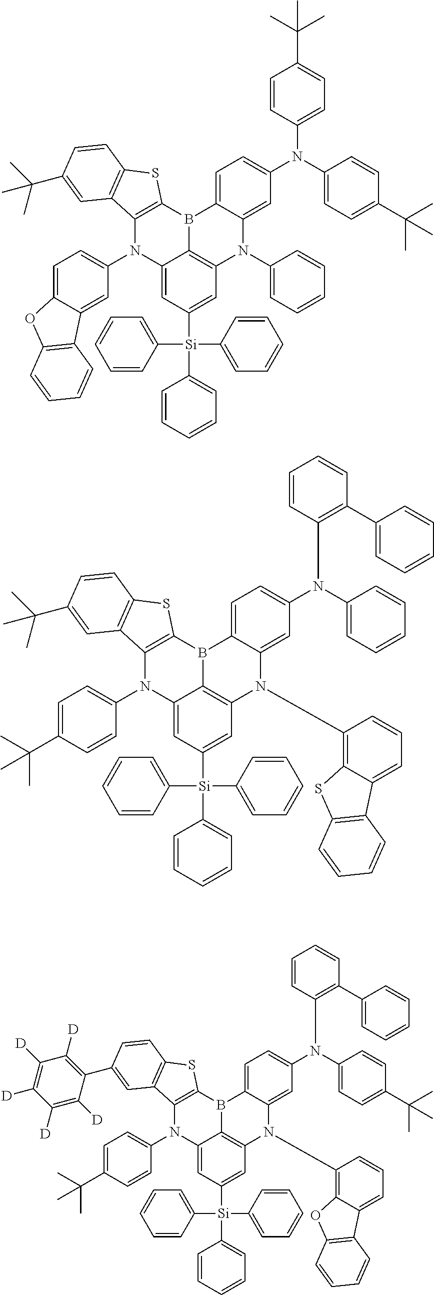

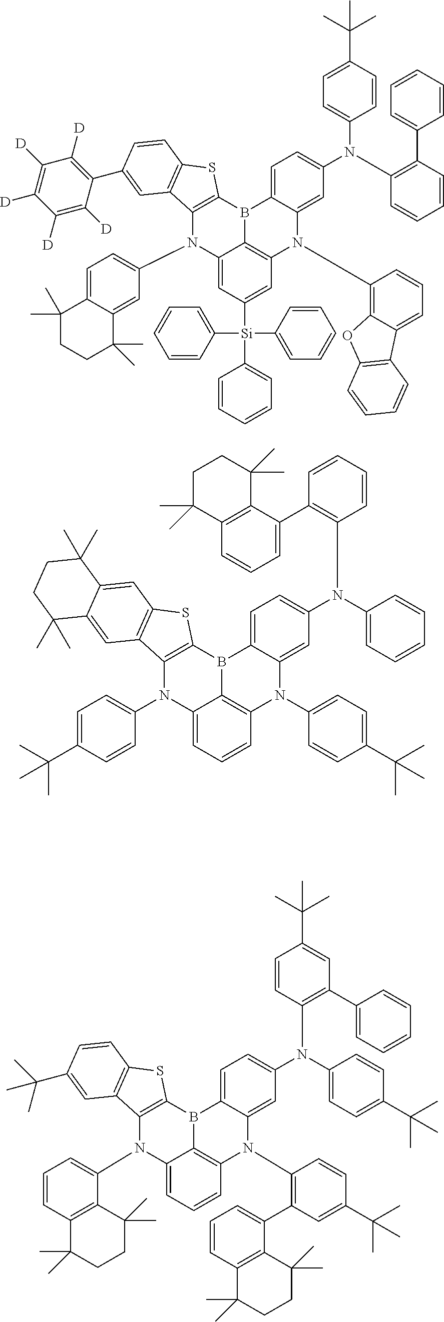

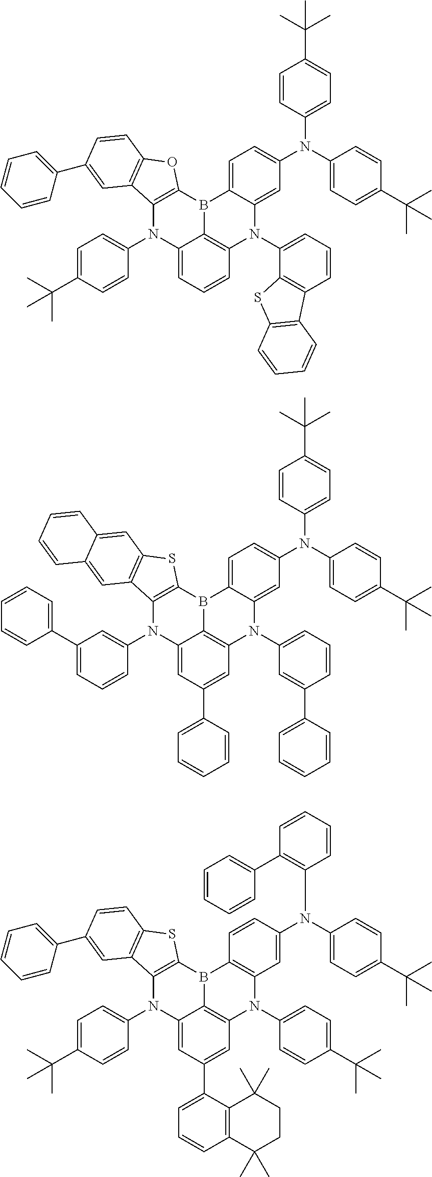

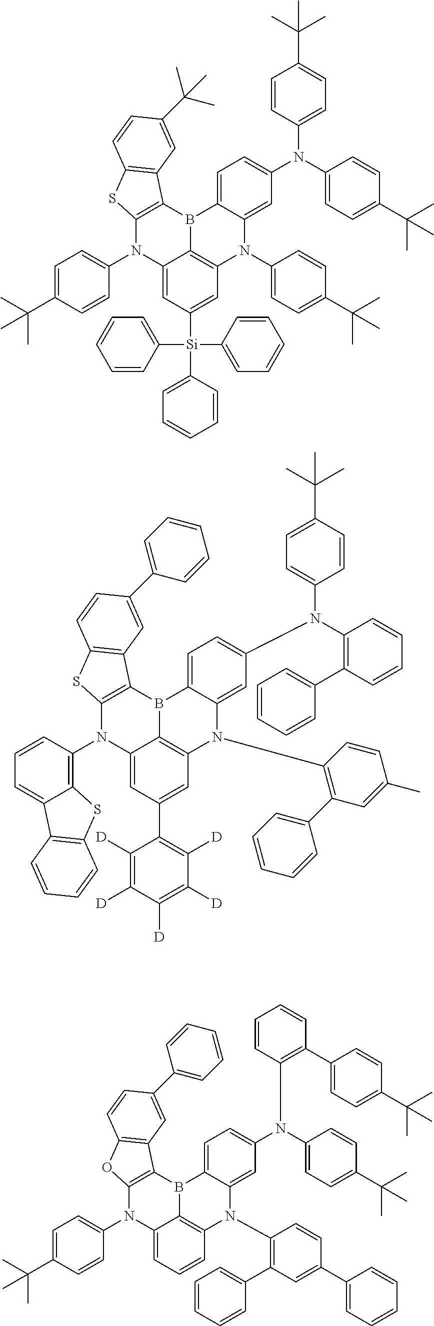

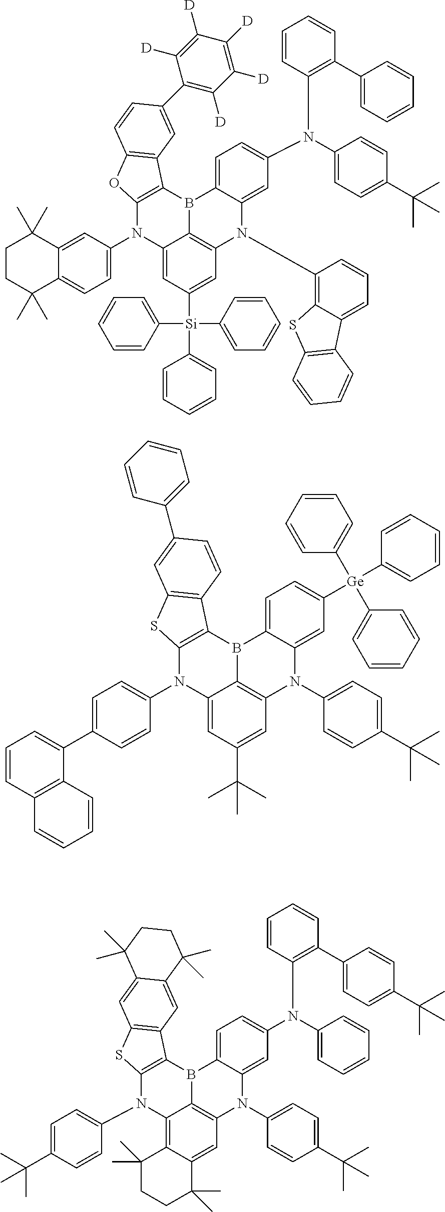

However, these compounds are not intended to limit the scope of Formula 2.

The organic layers of the organic light emitting device according to the present invention may form a monolayer structure. Alternatively, the organic layers may be stacked together to form a multilayer structure. For example, the organic layers may have a structure including a hole injecting layer, a hole transport layer, a hole blocking layer, a light emitting layer, an electron blocking layer, an electron transport layer, and an electron injecting layer but are not limited to this structure. The number of the organic layers is not limited and may be increased or decreased. Preferred structures of the organic layers of the organic light emitting device according to the present invention will be explained in more detail in the Examples section that follows.

A more detailed description will be given concerning exemplary embodiments of the organic light emitting device according to the present invention.

The organic light emitting device of the present invention includes an anode, a hole transport layer, a light emitting layer, an electron transport layer, and a cathode. The organic light emitting device of the present invention may optionally further include a hole injecting layer between the anode and the hole transport layer and an electron injecting layer between the electron transport layer and the cathode. If necessary, the organic light emitting device of the present invention may further include one or two intermediate layers such as a hole blocking layer or an electron blocking layer. The organic light emitting device of the present invention may further include one or more organic layers such as a capping layer that have various functions depending on the desired characteristics of the device.

A specific structure of the organic light emitting device according to one embodiment of the present invention, a method for fabricating the device, and materials for the organic layers are as follows.

First, an anode material is coated on a substrate to form an anode. The substrate may be any of those used in general organic light emitting devices. The substrate is preferably an organic substrate or a transparent plastic substrate that is excellent in transparency, surface smoothness, ease of handling, and waterproofness. A highly transparent and conductive metal oxide such as indium tin oxide (ITO), indium zinc oxide (IZO), tin oxide (SnO₂) or zinc oxide (ZnO) is used as the anode material.

A hole injecting material is coated on the anode by vacuum thermal evaporation or spin coating to form a hole injecting layer. Then, a hole transport material is coated on the hole injecting layer by vacuum thermal evaporation or spin coating to form a hole transport layer.

The hole injecting material is not specially limited so long as it is usually used in the art. Specific examples of such materials include 4,4′,4″-tris (2-naphthylphenyl-phenylamino) triphenylamine (2-TNATA), N,N′-di (1-naphthyl)-N,N′-diphenylbenzidine (NPD), N,N′-diphenyl-N,N′-bis(3-methylphenyl)-1,1′-biphenyl-4,4′-diamine (TPD), and N,N′-diphenyl-N,N′-bis(4-(phenyl-m-tolylamino) phenyl) biphenyl-4,4′-diamine (DNTPD).

The hole transport material is not specially limited so long as it is commonly used in the art. Examples of such materials include N,N′-bis(3-methylphenyl)-N,N′-diphenyl-(1,1-biphenyl)-4,4′-diamine (TPD) and N,N′-di (naphthalen-1-yl)-N,N′-diphenylbenzidine (a-NPD).

Subsequently, a hole auxiliary layer and a light emitting layer are sequentially stacked on the hole transport layer. A hole blocking layer may be optionally formed on the light emitting layer by vacuum thermal evaporation or spin coating. The hole blocking layer is formed as a thin film and blocks holes from entering a cathode through the organic light emitting layer. This role of the hole blocking layer prevents the lifetime and efficiency of the device from deteriorating. A material having a very low highest occupied molecular orbital (HOMO) energy level is used for the hole blocking layer. The hole blocking material is not particularly limited so long as it can transport electrons and has a higher ionization potential than the light emitting compound. Representative examples of suitable hole blocking materials include BAlq, BCP, and TPBI.

Examples of materials for the hole blocking layer include, but are not limited to, BAlq, BCP, Bphen, TPBI, TAZ, BeBq₂, OXD-7, and Liq.

An electron transport layer is deposited on the hole blocking layer by vacuum thermal evaporation or spin coating, and an electron injecting layer is formed thereon. A cathode metal is deposited on the electron injecting layer by vacuum thermal evaporation to form a cathode, completing the fabrication of the organic light emitting device.

For example, lithium (Li), magnesium (Mg), aluminum (Al), aluminum-lithium (Al—Li), calcium (Ca), magnesium-indium (Mg—In) or magnesium-silver (Mg—Ag) may be used as the metal for the formation of the cathode. The organic light emitting device may be of top emission type. In this case, a transmissive material such as ITO or IZO may be used to form the cathode.

A material for the electron transport layer functions to stably transport electrons injected from the cathode. The electron transport material may be any of those known in the art and examples thereof include, but are not limited to, quinoline derivatives, particularly tris (8-quinolinolato) aluminum (Alq3), TAZ, BAlq, beryllium bis(benzoquinolin-10-olate) (Bebq2), and oxadiazole derivatives such as PBD, BMD, and BND.

Each of the organic layers can be formed by a monomolecular deposition or solution process. According to the monomolecular deposition process, the material for each layer is evaporated into a thin film under heat and vacuum or reduced pressure. According to the solution process, the material for each layer is mixed with a suitable solvent and the mixture is then formed into a thin film by a suitable method such as ink-jet printing, roll-to-roll coating, screen printing, spray coating, dip coating or spin coating.

The organic light emitting device of the present invention can be used in a display or lighting system selected from flat panel displays, flexible displays, monochromatic flat panel lighting systems, white flat panel lighting systems, flexible monochromatic lighting systems, flexible white lighting systems, displays for automotive applications, displays for virtual reality, and displays for augmented reality.

### MODE FOR CARRYING OUT THE INVENTION

### Synthesis Example 1. Synthesis of BH-1

### Synthesis Example 1-1: Synthesis of A-1

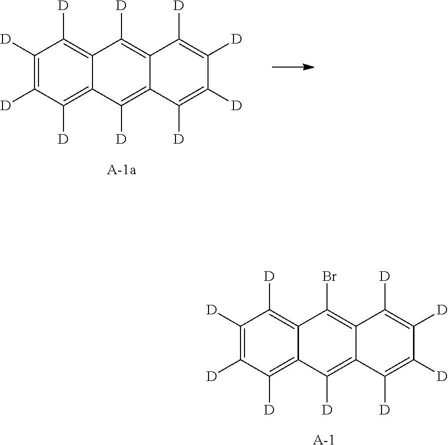

100 g of A-la and 800 mL of DMF were placed in a reactor and 103 g of NBS was slowly added dropwise thereto. After the dropwise addition, the mixture was stirred for 2 h. To the reaction mixture was added dropwise water. The resulting solid was collected by filtration, dissolved in dichloromethane, washed with distilled water, concentrated, crystallized from methanol, and dried to afford A-1 (129 g, 91.3%).

### Synthesis Example 1-2: Synthesis of A-2

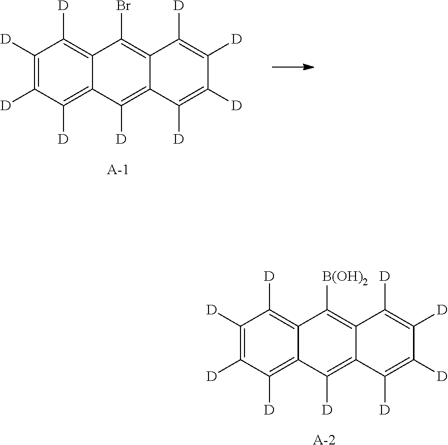

30 g of A-1 and 300 mL of tetrahydrofuran were dissolved in a reactor. The solution was cooled to −78° C. under a nitrogen atmosphere, followed by stirring. To the cooled reaction mixture was slowly added dropwise 124 mL of n-butyllithium. The mixture was stirred at the same temperature for 2 h. After slow dropwise addition of 15.2 g of trimethyl borate for 30 min, the resulting mixture was stirred at room temperature overnight. After completion of the reaction, the reaction mixture was acidified by slow dropwise addition of 2 N hydrochloric acid and extracted with water and ethyl acetate. The organic layer was separated and dried over magnesium sulfate. The residue was concentrated under reduced pressure and crystallized from heptane. The resulting solid was collected by filtration and washed with heptane and toluene to afford A-2 (16.3 g, 62.6%).

### Synthesis Example 1-3: Synthesis of A-3

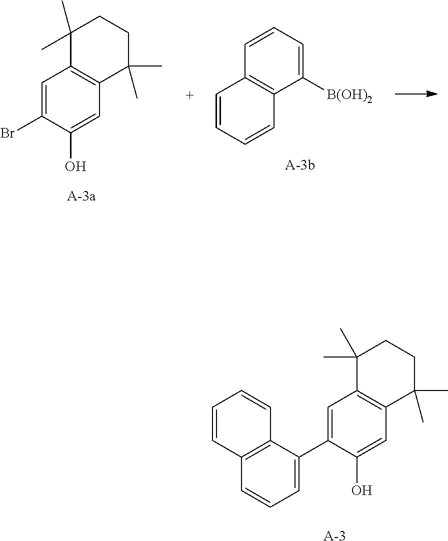

25 g of A-3a, 15.2 g of A-3b, 2 g of tetrakis (triphenylphosphine) palladium (0), 24.4 g of potassium carbonate, 200 mL of toluene, 200 mL of ethanol, and 100 mL of distilled water were stirred under reflux in a reactor. The reaction was allowed to proceed for one day. The completion of the reaction was confirmed by TLC. The reaction mixture was cooled to room temperature and extracted with distilled water and ethyl acetate. The organic layer was concentrated, purified by column chromatography, and recrystallized to afford A-3 (21.5 g, 73.7%).

### Synthesis Example 1-4: Synthesis of A-4

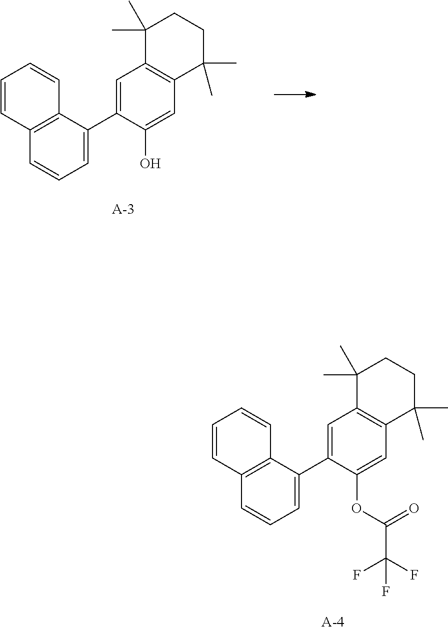 [SMILES: CC1(C)CCC(C)(C)c2cc(-c3cccc4ccccc34)c(O)cc21 ; "A-3" ; CC1(C)CCC(C)(C)c2cc(-c3cccc4ccccc34)c(OC(=O)C(F)(F)F)cc21 ; "A-4"]

10 g of A-3 and 150 mL of dichloromethane were placed in a reactor, and 7.3 g of trifluoroacetic anhydride and 4 g of triethylamine were slowly added dropwise thereto with stirring. Thereafter, stirring was continued for 3 h. The reaction mixture was extracted with distilled water and dichloromethane. The organic layer was separated, dried over magnesium sulfate, concentrated in vacuo, and purified by column chromatography to afford A-4 (10.3 g, 79.8%).

### Synthesis Example 1-5: Synthesis of A-5

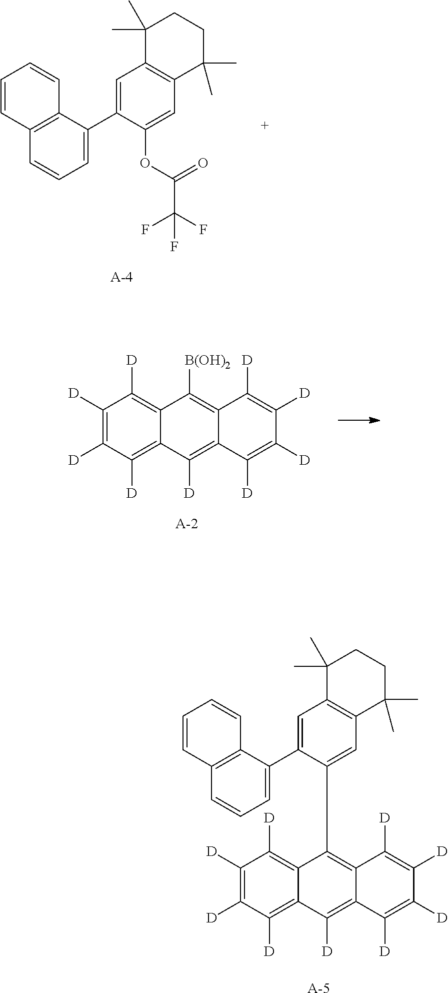

6 g of A-4 and 3.6 g of A-2 were dissolved in 250 mL of THF in a reactor, and then a 2 M aqueous potassium carbonate solution and 0.33 g of tetrakis (triphenylphosphine) palladium (0) were added thereto. The mixture was stirred under heating for 6 h. After completion of the reaction, the temperature was lowered to room temperature. The resulting solid was collected by filtration, dried, purified by column chromatography, and recrystallized to afford A-5 (4 g, 56.7%).

### Synthesis Example 1-6: Synthesis of A-6

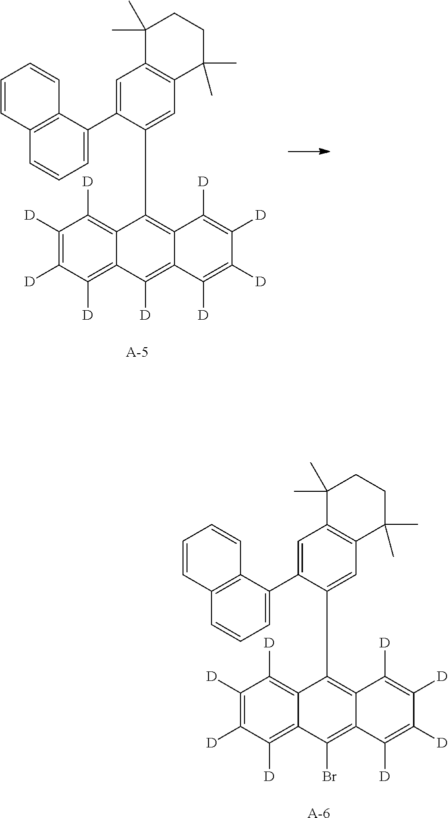

A-6 (yield 82.7%) was synthesized in the same manner as in Synthesis Example 1-1, except that A-5 was used instead of A-la.

### Synthesis Example 1-7: Synthesis of BH-1

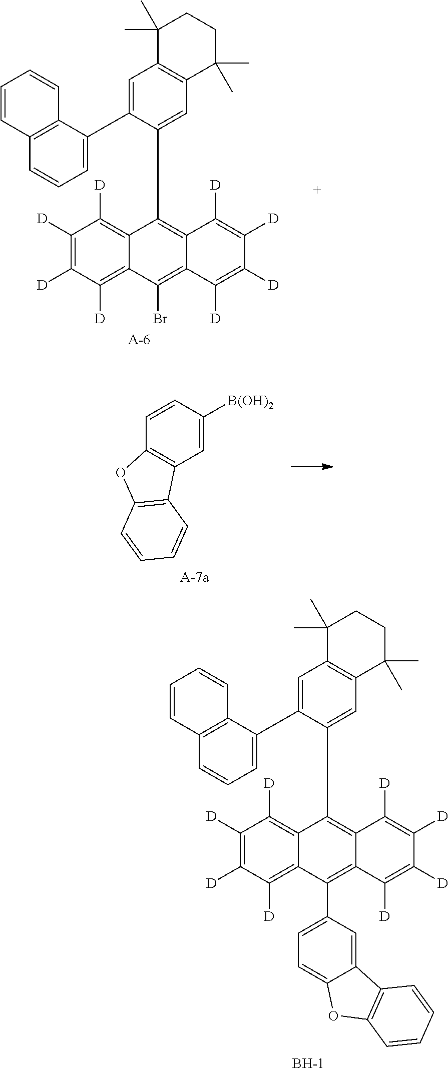

20 g of A-6, 9.5 g of A-7a, 0.2 g of palladium (II) acetate, 10 g of sodium tert-butoxide, 0.5 g of BIDIME, and 210 mL of toluene were stirred under reflux in a reactor for 3 days. After the completion of the reaction was confirmed by TLC, the reaction temperature was lowered to room temperature. The reaction mixture was extracted with distilled water and ethyl acetate. The organic layer was concentrated, purified by column chromatography, and recrystallized to afford BH-1 (12 g, 52.1%).

MS (MALDI-TOF): m/z 664.36 [M⁺]

### Synthesis Example 2. Synthesis of BH-2

### Synthesis Example 2-1: Synthesis of B-1

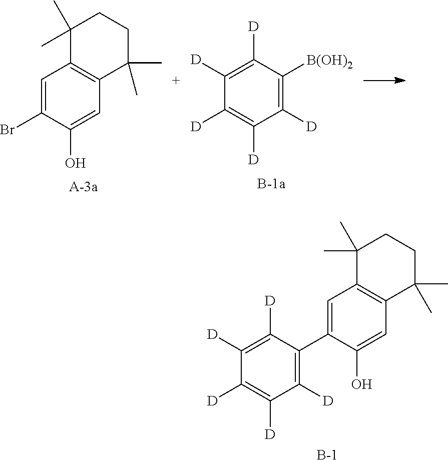

B-1 (yield 84%) was synthesized in the same manner as in Synthesis Example 1-3, except that B-la was used instead of A-3b.

### Synthesis Example 2-2: Synthesis of B-2

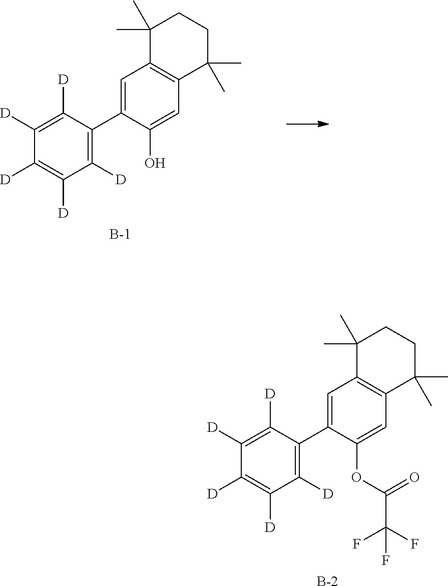

B-2 (yield 82.3%) was synthesized in the same manner as in Synthesis Example 1-4, except that B-1 was used instead of A-3.

### Synthesis Example 2-3: Synthesis of B-3

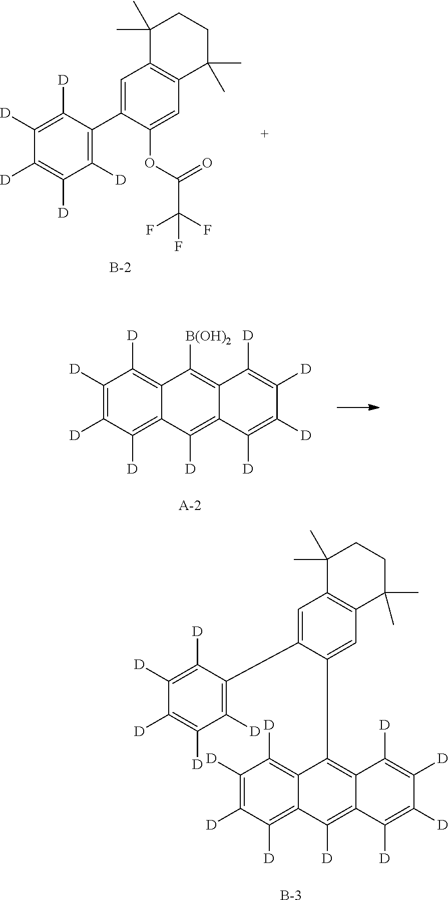

B-3 (yield 60.1%) was synthesized in the same manner as in Synthesis Example 1-5, except that B-2 was used instead of A-4.

### Synthesis Example 2-4: Synthesis of B-4

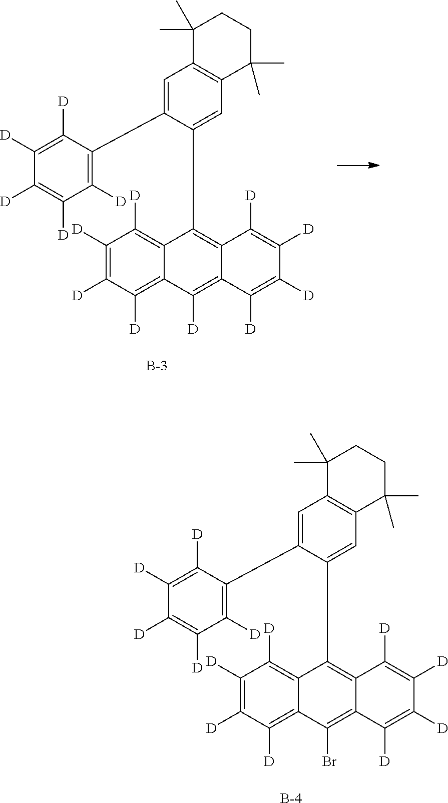

B-4 (yield 92.1%) was synthesized in the same manner as in Synthesis Example 1-6, except that B-3 was used instead of A-5.

### Synthesis Example 2-5: Synthesis of BH-2

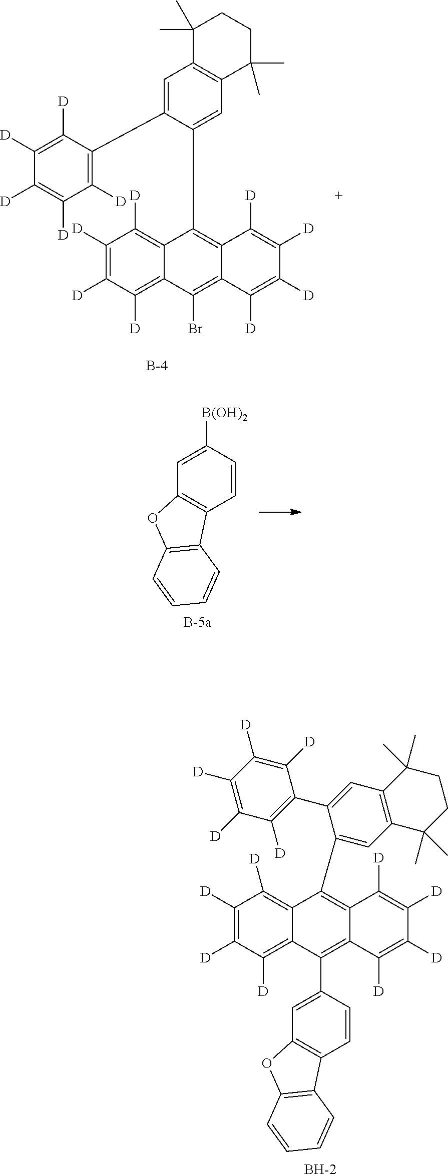

BH-2 (yield 56.1%) was synthesized in the same manner as in Synthesis Example 1-7, except that B-4 and B-Sa were used instead of A-6 and A-7a, respectively. MS (MALDI-TOF): m/z 619.37 [M⁺]

### Synthesis Example 3. Synthesis of BH-3

### Synthesis Example 3-1: Synthesis of C-1

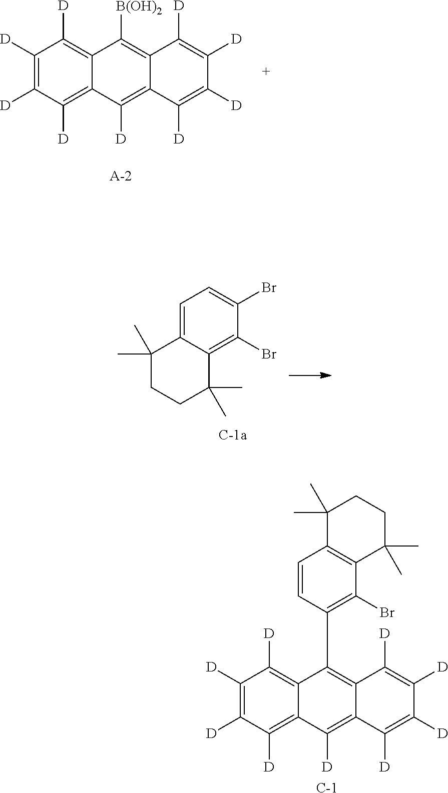

C-1 (yield 87%) was synthesized in the same manner as in Synthesis Example 1-3, except that C-1a and A-2 were used instead of A-3a and A-3b, respectively.

### Synthesis Example 3-2: Synthesis of C-2

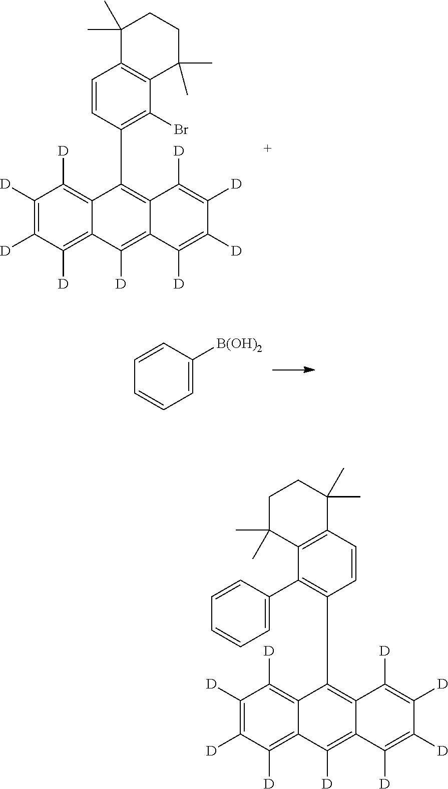 [SMILES: [2H]c1c([2H])c([2H])c2c(-c3ccc4c(c3Br)C(C)(C)CCC4(C)C)c3c([2H])c([2H])c([2H])c([2H])c3c([2H])c2c1[2H] ; OB(O)c1ccccc1 ; [2H]c1c([2H])c([2H])c2c(-c3ccc4c(c3-c3ccccc3)C(C)(C)CCC4(C)C)c3c([2H])c([2H])c([2H])c([2H])c3c([2H])c2c1[2H]]

C-2 (yield 84.6%) was synthesized in the same manner as in Synthesis Example 1-3, except that C-1 and C-2a were used instead of A-3a and A-3b, respectively.

### Synthesis Example 3-3: Synthesis of C-3

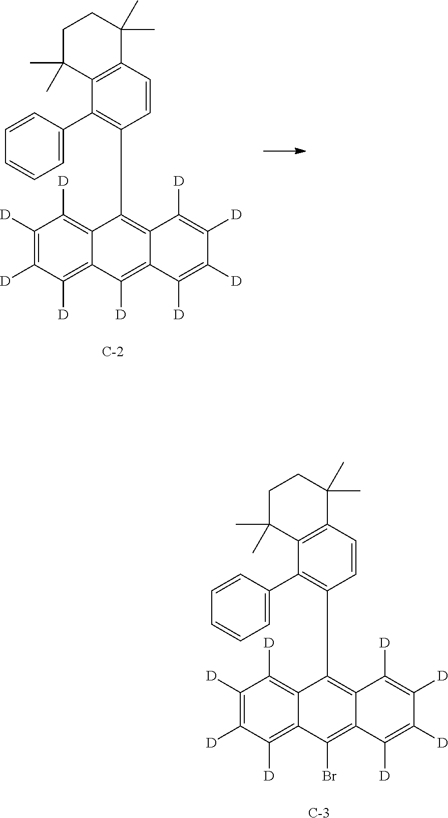

C-3 (yield 93.2%) was synthesized in the same manner as in Synthesis Example 1-1, except that C-2 was used instead of A-la.

### Synthesis Example 3-4: Synthesis of BH-3

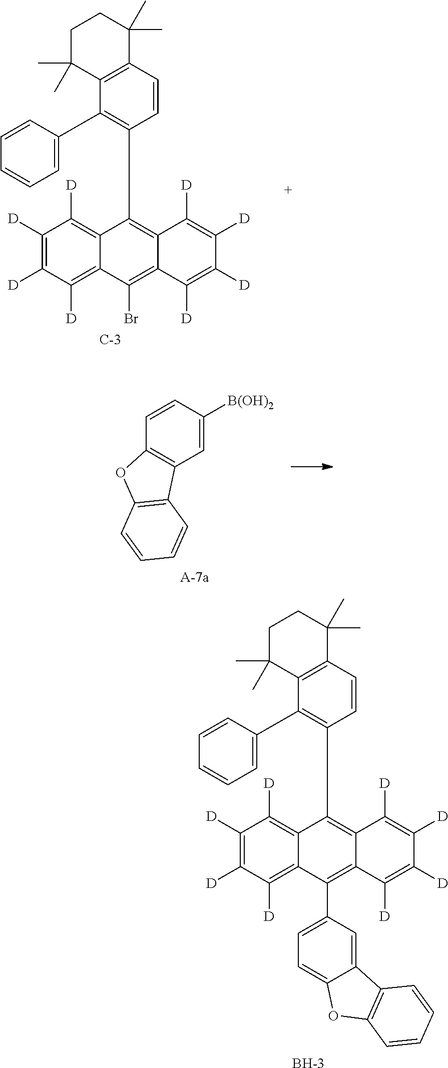

BH-3 (yield 57.6%) was synthesized in the same manner as in Synthesis Example 1-7, except that C-3 was used instead of A-6.

MS (MALDI-TOF): m/z 614.34 [M⁺]

### Synthesis Example 4. Synthesis of BH-4

### Synthesis Example 4-1: Synthesis of D-1

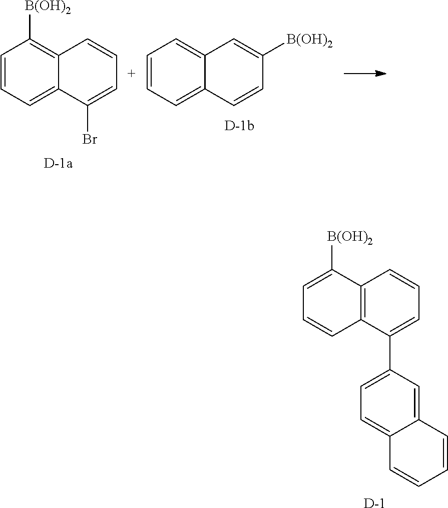

D-1 (yield 79.2%) was synthesized in the same manner as in Synthesis Example 1-3, except that D-1a and D-1b were used instead of A-3a and A-3b, respectively.

### Synthesis Example 4-2: Synthesis of D-2

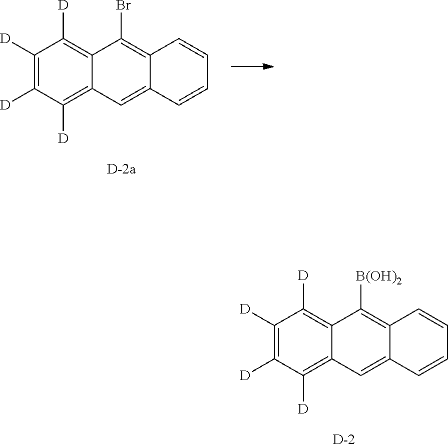

D-2 (yield 69.3%) was synthesized in the same manner as in Synthesis Example 1-2, except that D-2a was used instead of A-1.

### Synthesis Example 4-3: Synthesis of D-3

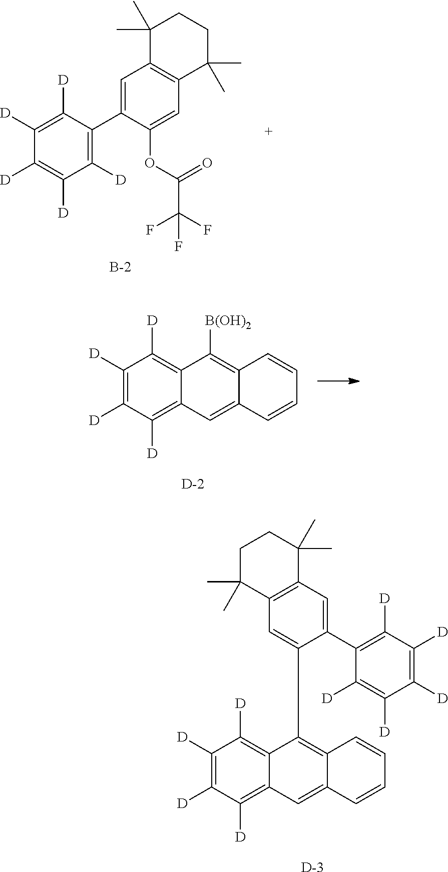

D-3 (yield 57.1%) was synthesized in the same manner as in Synthesis Example 1-5, except that B-2 and D-2 were used instead of A-4 and A-2, respectively.

### Synthesis Example 4-4: Synthesis of D-4

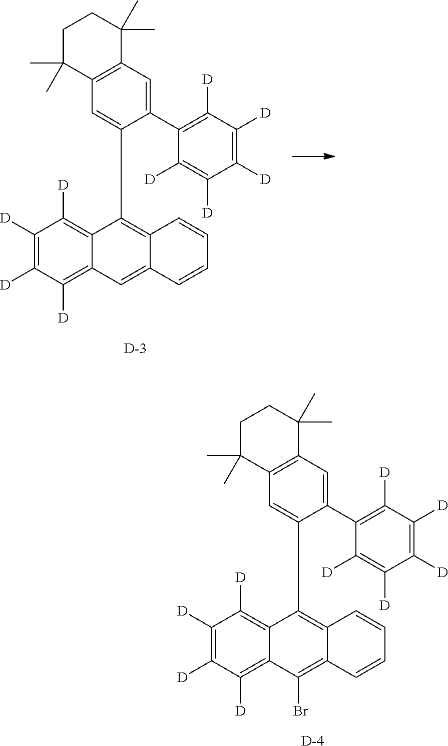

D-4 (yield 87.6%) was synthesized in the same manner as in Synthesis Example 1-1, except that D-3 was used instead of A-la.

### Synthesis Example 4-5: Synthesis of BH-4

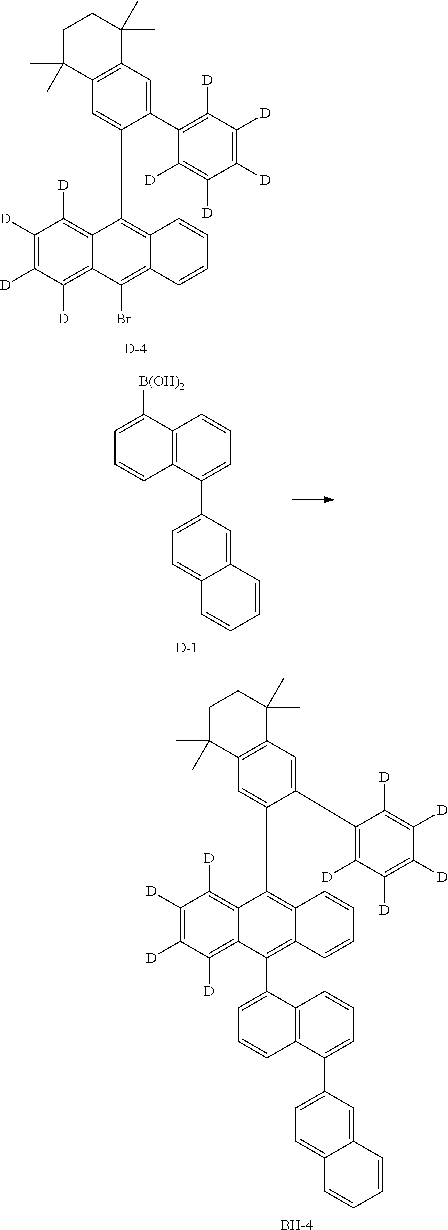

BH-4 (yield 54.4%) was synthesized in the same manner as in Synthesis Example 1-7, except that D-4 and D-1 were used instead of A-6 and A-7a, respectively.

MS (MALDI-TOF): m/z 701.40 [M⁺]

### Synthesis Example 5. Synthesis of BH-5

### Synthesis Example 5-1: Synthesis of BH-5

BH-5 was synthesized in the same manner as in Synthesis Example 1-3, except that pyridin-3-ylboronic acid was used instead of A-3b.

MS (MALDI-TOF): m/z 615.34 [M⁺]

### Synthesis Example 6. Synthesis of BH-6

### Synthesis Example 6-1: Synthesis of F-1

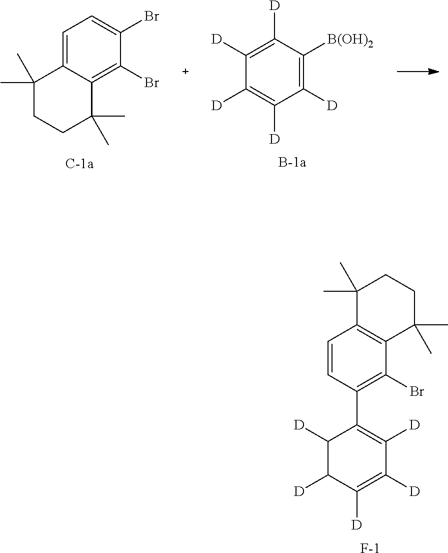

F-1 (yield 86.9%) was synthesized in the same manner as in Synthesis Example 1-3, except that C-1a and B-la were used instead of A-3a and A-3b, respectively.

### Synthesis Example 6-2: Synthesis of F-2

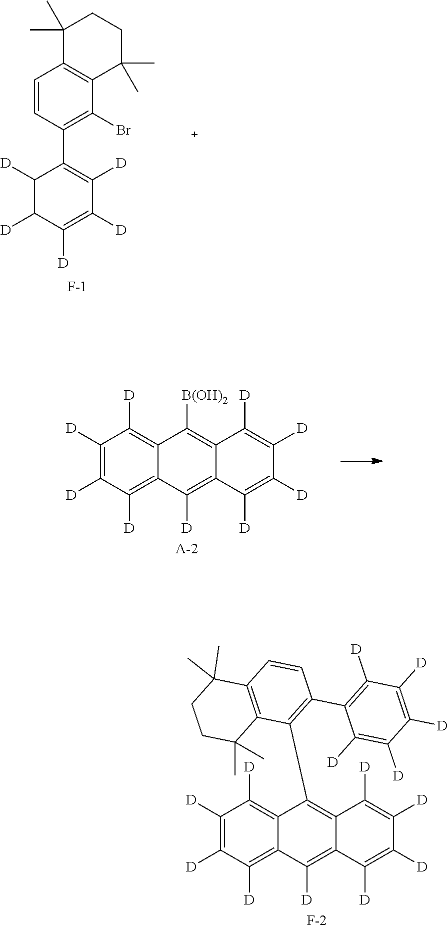

F-2 (yield 83.1%) was synthesized in the same manner as in Synthesis Example 1-3, except that F-1 and A-2 were used instead of A-3a and A-3b, respectively.

### Synthesis Example 6-3: Synthesis of F-3

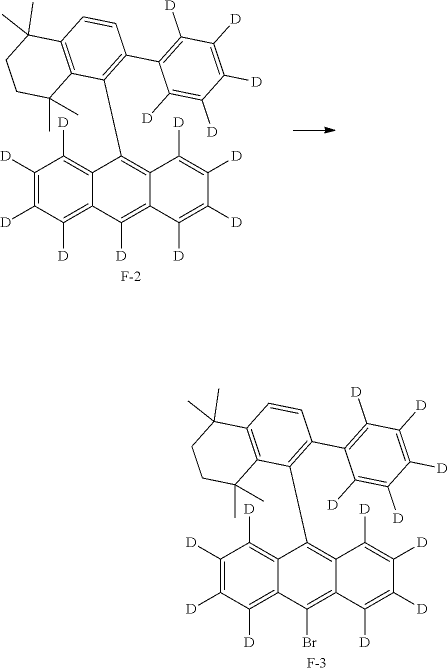

F-3 (yield 89.4%) was synthesized in the same manner as in Synthesis Example 1-1, except that F-2 was used instead of A-la.

### Synthesis Example 6-4: Synthesis of BH-6

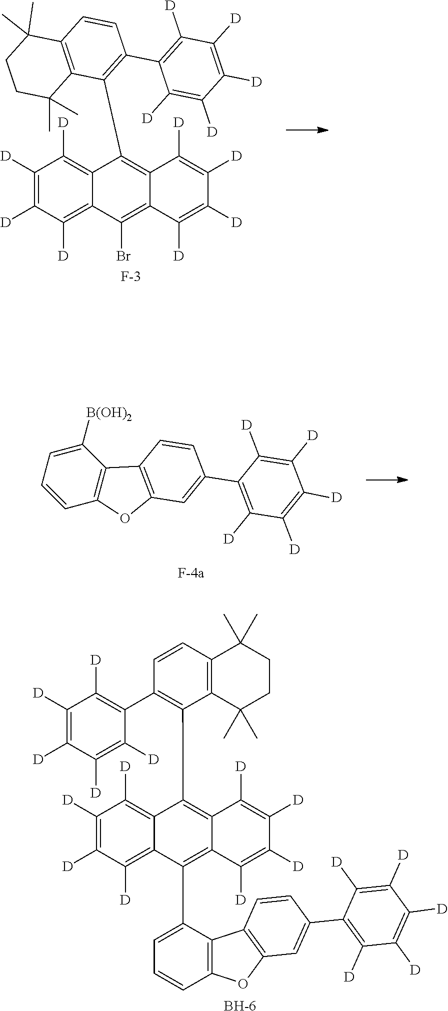

BH-6 (yield 50.8%) was synthesized in the same manner as in Synthesis Example 1-7, except that F-3 and F-4a were used instead of A-6 and A-7a, respectively.

MS (MALDI-TOF): m/z 700.44 [M⁺]

### Synthesis Example 7. Synthesis of BH-7

### Synthesis Example 7-1: Synthesis of BH-7

BH-7 (final yield 51.2%) was synthesized in the same manner as in Synthesis Example 3, except that phenanthren-9-ylboronic acid was used instead of A-7a in Synthesis Example 3-4.

MS (MALDI-TOF): m/z 624.36 [M⁺]

### Synthesis Example 8. Synthesis of BH-8

### Synthesis Example 8-1: Synthesis of BH-8

BH-8 (final yield 49.4%) was synthesized in the same manner as in Synthesis Example 2, except that dibenzo[b,d] thiophen-4-ylboronic acid was used instead of B-5a in Synthesis Example 2-5.

MS (MALDI-TOF): m/z 635.35 [M⁺]

### Synthesis Example 9. Synthesis of BD-1

### Synthesis Example 9-1: Synthesis of H-1

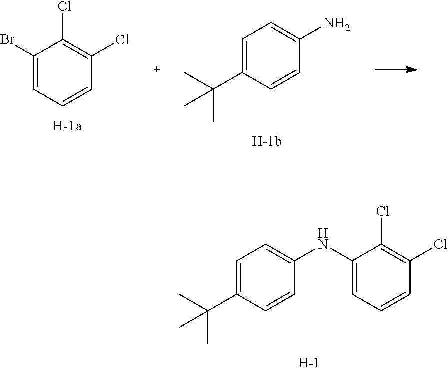

30 g of H-1a, 25.8 g of H-1b, 0.9 g of palladium acetate, 25.5 g of sodium tert-butoxide, 2.5 g of bis(diphenylphosphino)-1,1′-binaphthyl, and 400 mL of toluene were stirred under reflux in a reactor for 24 h. After completion of the reaction, the reaction mixture was filtered, concentrated, and purified by column chromatography to afford H-1 (32 g, 81.9%).

### Synthesis Example 9-2: Synthesis of H-2

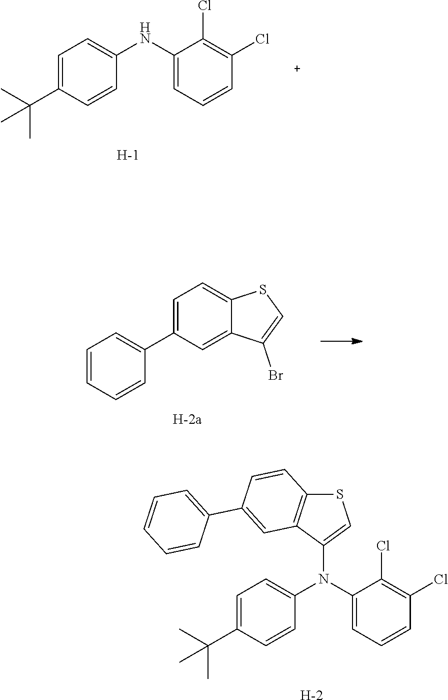

H-2 (yield 73.2%) was synthesized in the same manner as in Synthesis Example 9-1, except that H-2a and H-1 were used instead of H-1a and H-1b, respectively.

### Synthesis Example 9-3: Synthesis of H-3

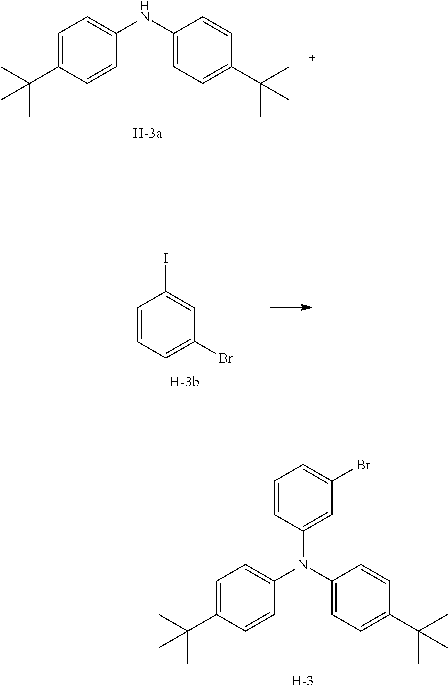

H-3 (yield 76.9%) was synthesized in the same manner as in Synthesis Example 9-1, except that H-3b and H-3a were used instead of H-1a and H-1b, respectively.

### Synthesis Example 9-4: Synthesis of H-4

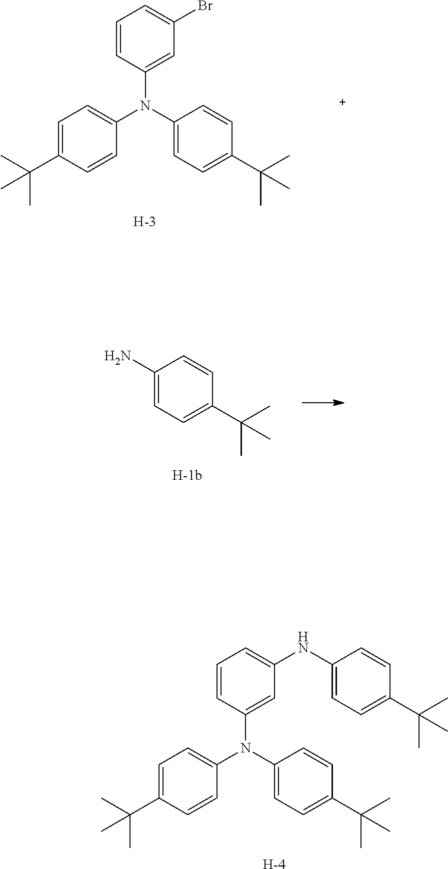

H-4 (yield 72.7%) was synthesized in the same manner as in Synthesis Example 9-1, except that H-3 was used instead of H-1a.

### Synthesis Example 9-5: Synthesis of H-5

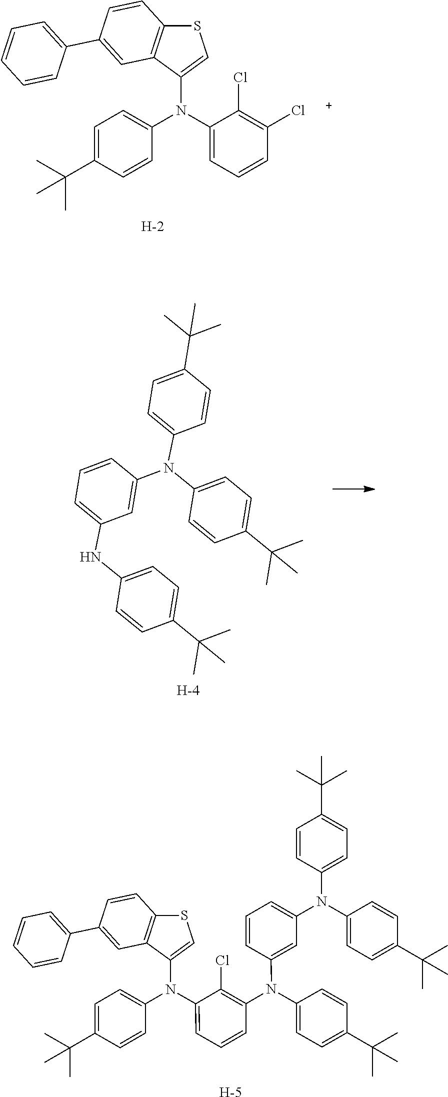

H-5 (yield 65.9%) was synthesized in the same manner as in Synthesis Example 9-1, except that H-2 and H-4 were used instead of H-1a and H-1b, respectively.

### Synthesis Example 9-6: Synthesis of BD-1

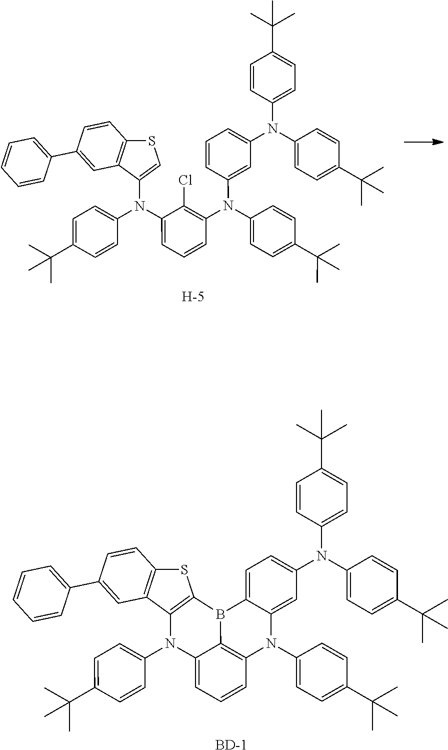

20 g of H-5 and tert-butylbenzene were placed in a reactor, and 36 mL of tert-butyllithium was added dropwise thereto at −78° C. After the dropwise addition, the mixture was stirred at 60° C. for 3 h. Thereafter, nitrogen was blown into the mixture to remove pentane. After cooling to −78° C., 4 mL of boron tribromide was added dropwise.

The resulting mixture was stirred at room temperature for 1 h. After dropwise addition of 5.3 g of N,N-diisopropylethylamine at 0° C., stirring was continued at 120° C. for 2 h. After completion of the reaction, the reaction mixture was added with an aqueous sodium acetate solution, stirred, and extracted with ethyl acetate. The organic layer was concentrated and purified by column chromatography to afford BD-1 (2.8 g, 14.4%).

MS (MALDI-TOF): m/z 943.51 [M⁺]

### Synthesis Example 10. Synthesis of BD-2

### Synthesis Example 10-1: Synthesis of I-1

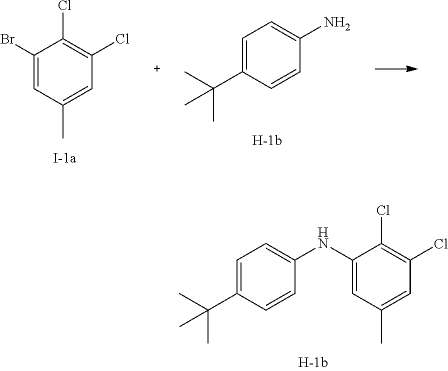

I-1 (yield 79.8%) was synthesized in the same manner as in Synthesis Example 9-1, except that I-la was used instead of H-1a.

### Synthesis Example 10-2: Synthesis of I-2

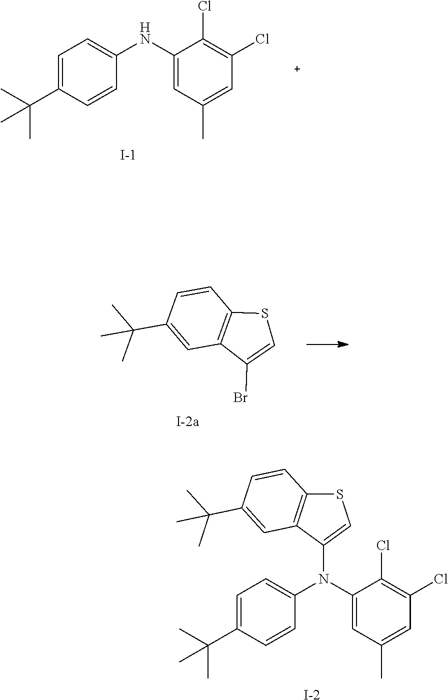

I-2 (yield 69.7%) was synthesized in the same manner as in Synthesis Example 9-1, except that I-2a and I-1 were used instead of H-1a and H-1b, respectively.

### Synthesis Example 10-3: Synthesis of I-3

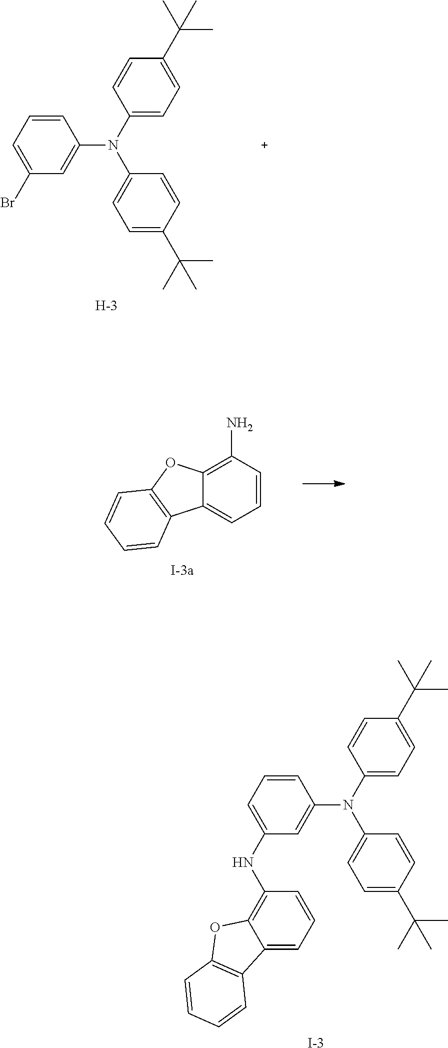

I-3 (yield 70.3%) was synthesized in the same manner as in Synthesis Example 9-1, except that H-3 and I-3a were used instead of H-1a and H-1b, respectively.

### Synthesis Example 10-4: Synthesis of I-4

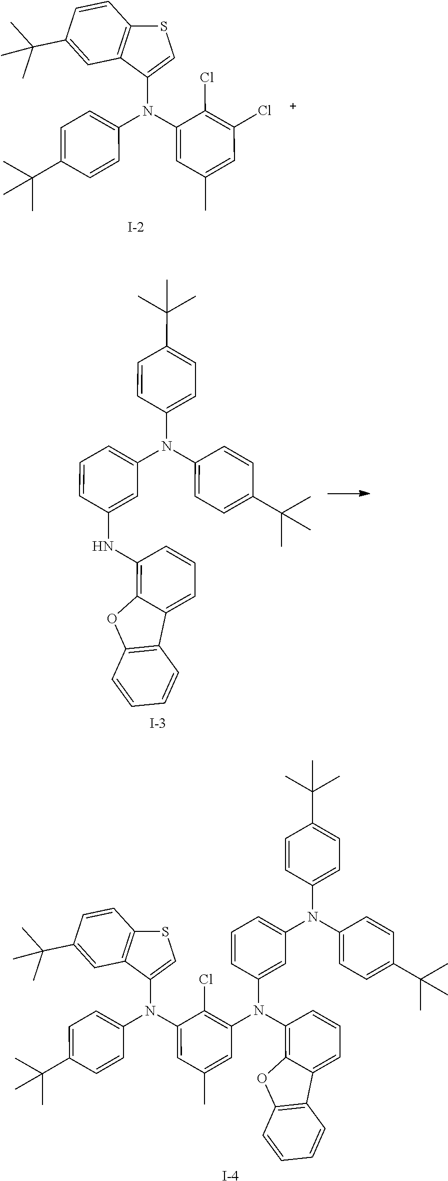

I-4 (yield 76.7%) was synthesized in the same manner as in Synthesis Example 9-1, except that I-2 and I-3 were used instead of H-1a and H-1b, respectively.

### Synthesis Example 10-5: Synthesis of BD-2

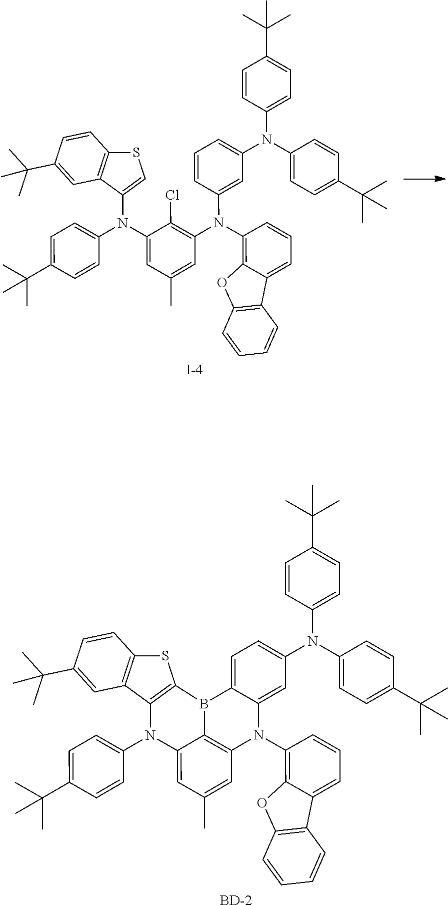

BD-2 (yield 17.4%) was synthesized in the same manner as in Synthesis Example 9-6, except that I-4 was used instead of H-5.

MS (MALDI-TOF): m/z 971.5 [M⁺]

### Examples 1-11: Fabrication of organic light emitting devices

ITO glass was patterned to have a light emitting area of 2 mm×2 mm, followed by cleaning. After the cleaned ITO glass was mounted in a vacuum chamber, the base pressure was adjusted to 1×10⁻⁷ torr. 2-TNATA (400 Å) and HT (200 Å) were deposited in this order on the ITO glass. The inventive host compound shown in Table 1 was mixed with 3 wt % of the inventive dopant compound BD-1 or BD-2. The mixture was used to form a 250 Å thick light emitting layer. Then, the compound of Formula E-1 and Liq were sequentially deposited to form a 300 Å thick electron transport layer and a 10 Å electron injecting layer on the light emitting layer, and A1 (1,000 Å) was deposited thereon to form a cathode, completing the fabrication of an organic light emitting device. The luminescent properties of the organic light emitting device were measured at 10 mA/cm².

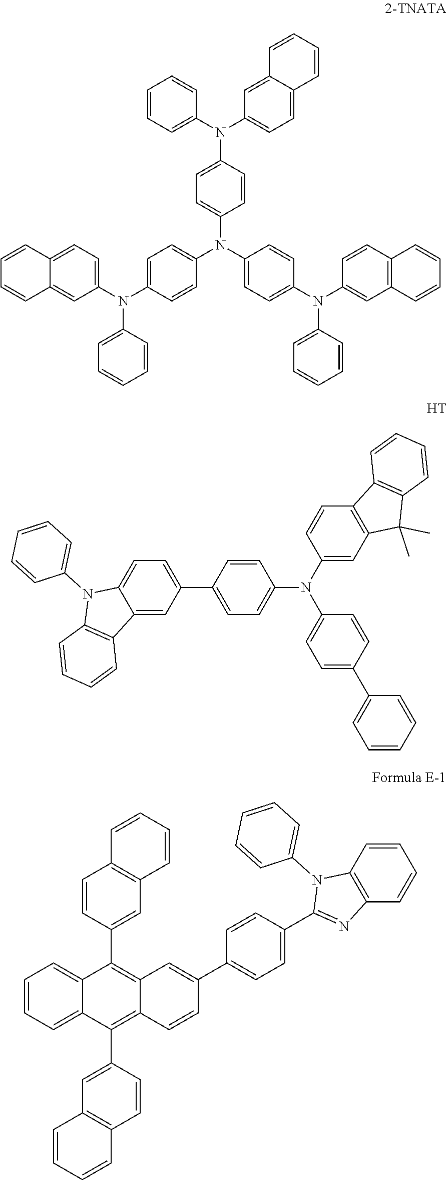 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

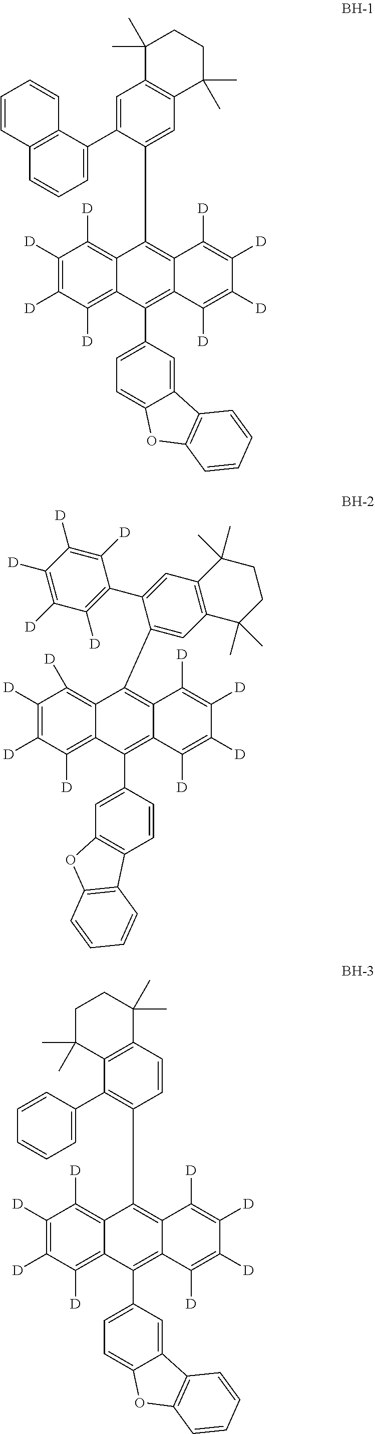

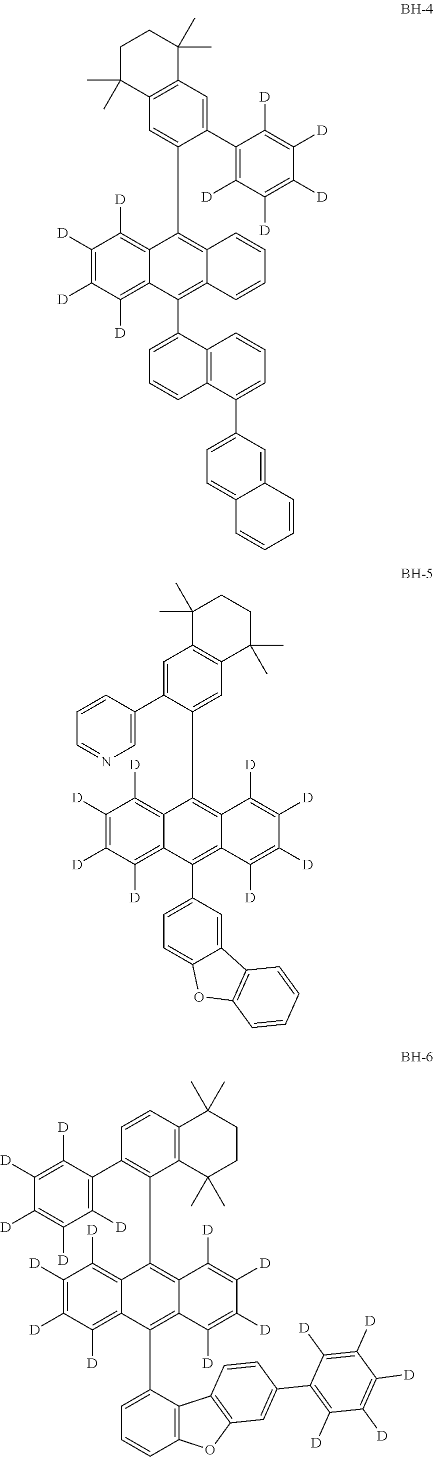 [SMILES: [2H]c1c([2H])c([2H])c(-c2cc3c(cc2-c2c4ccccc4c(-c4cccc5c(-c6ccc7ccccc7c6)cccc45)c4c([2H])c([2H])c([2H])c([2H])c24)C(C)(C)CCC3(C)C)c([2H])c1[2H] ; [2H]c1c([2H])c([2H])c2c(-c3cc4c(cc3-c3cccnc3)C(C)(C)CCC4(C)C)c3c([2H])c([2H])c([2H])c([2H])c3c(-c3ccc4oc5ccccc5c4c3)c2c1[2H] ; [2H]c1c([2H])c([2H])c(-c2ccc3c(c2)oc2cccc(-c4c5c([2H])c([2H])c([2H])c([2H])c5c(-c5c(-c6c([2H])c([2H])c([2H])c([2H])c6[2H])ccc6c5C(C)(C)CCC6(C)C)c5c([2H])c([2H])c([2H])c([2H])c45)c23)c([2H])c1[2H]]

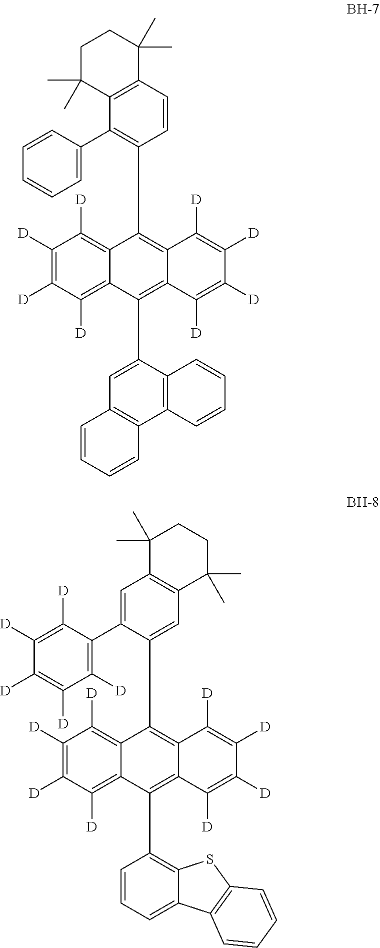

### Comparative Examples 1-7

Organic light emitting devices were fabricated in the same manner as in Examples 1-11, except that RH-1, RH-2, RH-3 or RH-4 was used as a host compound instead of the inventive compound and RD-1 was used as a dopant compound. The luminescent properties of the organic light emitting devices were measured at 10 mA/cm².

The structures of RH-1 to RH-4 and RD-2 are as follow:

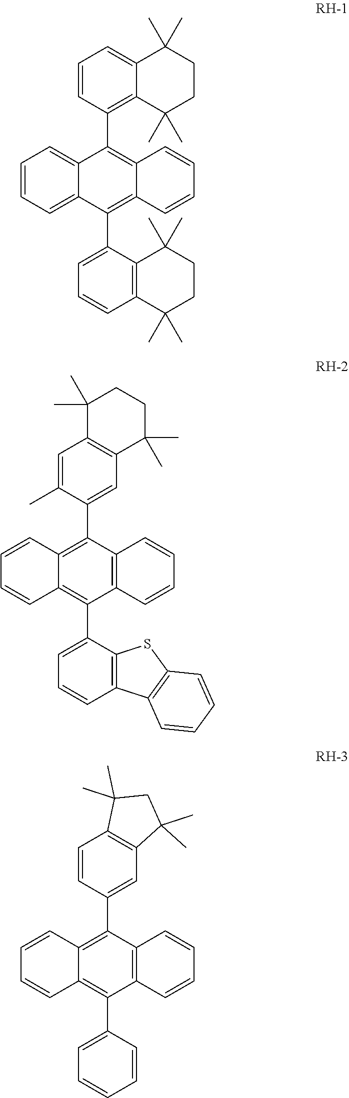

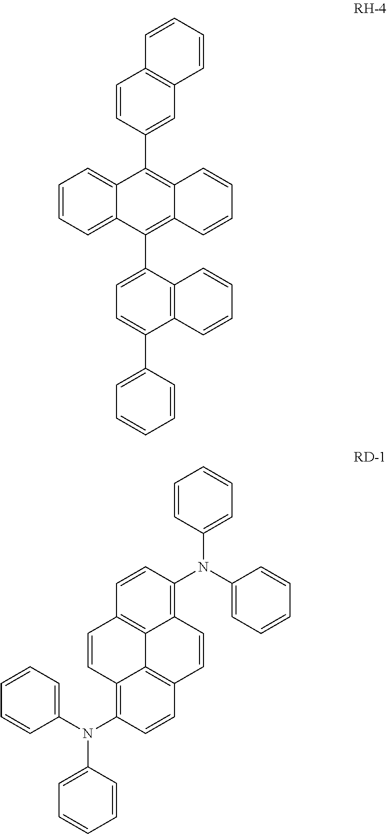

TABLE 1

| | | | External | |
|---|---|---|---|---|
| | | | quantum | Lifetime |
| Example No. | Host | Dopant | efficiency (%) | (T97, hr) |
| Example 1 | BH-1 | BD-1 | 7.7 | 172 |
| Example 2 | BH-2 | BD-1 | 8.1 | 185 |
| Example 3 | BH-3 | BD-1 | 8.3 | 176 |
| Example 4 | BH-4 | BD-1 | 7.6 | 164 |
| Example 5 | BH-5 | BD-1 | 7.4 | 146 |
| Example 6 | BH-6 | BD-1 | 8.1 | 175 |
| Example 7 | BH-7 | BD-1 | 7.5 | 181 |
| Example 8 | BH-8 | BD-1 | 7.1 | 145 |
| Example 9 | BH-2 | BD-2 | 8.5 | 188 |
| Example 10 | BH-3 | BD-2 | 8.8 | 172 |
| Example 11 | BH-7 | BD-2 | 8.0 | 174 |
| Comparative Example 1 | RH-1 | BD-1 | 5.2 | 53 |
| Comparative Example 2 | RH-2 | BD-1 | 6.4 | 98 |
| Comparative Example 3 | RH-3 | BD-1 | 4.8 | 38 |
| Comparative Example 4 | RH-4 | BD-1 | 6.2 | 87 |
| Comparative Example 5 | RH-2 | BD-2 | 6.6 | 93 |
| Comparative Example 6 | RH-3 | BD-2 | 5.1 | 35 |
| Comparative Example 7 | RH-2 | RD-1 | 5.5 | 52 |

As can be seen from the results in Table 1, the organic light emitting devices of Examples 1-11, each of which employed the inventive compound represented by Formula 1 as a host and the inventive compound represented by Formula 2 as a dopant in the light emitting layer, had significantly improved external quantum efficiencies and life characteristics compared to the organic light emitting devices of Comparative Examples 1-7, each of which employed the compounds whose specific structures are contrasted with those of the inventive compounds.

Molecules used in layers during the fabrication of OLEDs pile up to form crystals and are recrystallized by heat generated during deposition. This phenomenon causes the layers to crack, resulting in short lifetime of the devices. In consideration of the phenomenon of recrystallization, the anthracene derivative of the present invention has been designed in which a bulkier substituent such as an aryl group is introduced at a specific position instead of hydrogen or a linear alkyl group in a mixed aliphatic-aromatic cyclic group at the 9- or 10-position of anthracene to impede regular arrangement of the molecules, and as a result, the intermolecular bonding strength is lowered, making it difficult to form crystals. This design increases the proportion of amorphous domains to suppress a reduction in lifetime while preventing excitons from being quenched by 2- and 3-dimensional intermolecular interactions, achieving an increase in luminous efficiency.

In addition, the replacement of some hydrogen atoms at specific positions of anthracene in the compound of the present invention or in the substituents of the compound by deuterium atoms leads to a further increase in lifetime, which compensates for the disadvantage of short lifetime observed in the undeuterated moiety.

### INDUSTRIAL APPLICABILITY

The organic light emitting device of the present invention includes a light emitting layer employing the anthracene derivative as a host and the polycyclic compound with a specific structure as a dopant. The use of the host and the dopant ensures significantly improved luminous efficiency and life characteristics of the device. Due to these advantages, the highly efficient and long-lasting organic light emitting device can be used in a display or lighting system selected from flat panel displays, flexible displays, monochromatic flat panel lighting systems, white flat panel lighting systems, flexible monochromatic lighting systems, flexible white lighting systems, displays for automotive applications, displays for virtual reality, and displays for augmented reality.

1. An anthracene derivative represented by Formula 1: 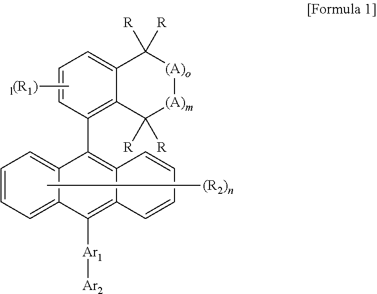
wherein the moieties A are identical to or different from each other and are each independently selected from O, S, and CR₃R₄, R, R₃, and R₄ are identical to or different from each other and are each independently selected from hydrogen, deuterium, and substituted or unsubstituted C₁-C₃₀ alkyl, with the proviso that adjacent ones of R, R₃, and R₄ are optionally linked to each other or each of R, R₃, and R₄ is optionally linked to an adjacent substituent to form an alicyclic or aromatic monocyclic or polycyclic ring, the groups R₁ are identical to or different from each other and are each independently selected from hydrogen, deuterium, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted C₃-C₅₀ mixed aliphatic-aromatic cyclic groups, the groups R₂ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, Ar₁ represents a single bond or is substituted or unsubstituted C₆-C₅₀ arylene or substituted or unsubstituted C₂-C₅₀ heteroarylene, Ar₂ is selected from substituted or unsubstituted C₆-C₅₀ aryl, substituted or unsubstituted C₃-C₅₀ cycloalkyl, substituted or unsubstituted C₂-C₅₀ heterocycloalkyl, and substituted or unsubstituted C₂-C₅₀ heteroaryl, 1 is an integer of 3, the groups R₁ are identical to or different from each other, with the proviso that at least one of the groups R₁ is not hydrogen or deuterium, each of o and m is independently an integer of 1 or 2, provided that when o or m is 2 or more, the moieties A are identical to or different from each other, n is an integer of 8, and the groups R₂ are identical to or different from each other, the “substituted” in the definitions of A, R, R₁, R₂, Ar₁, and Ar₂ in Formula 1 indicating substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, C₃-C₃₀ mixed aliphatic-aromatic cyclic groups, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof, the “unsubstituted” in the same definition indicating having no substituent, and one or more hydrogen atoms in each of the substituents being optionally replaced by deuterium atoms.

2. The anthracene derivative according to claim 1, wherein the anthracene derivative of Formula 1 is represented by Formula 1-1 or 1-2: 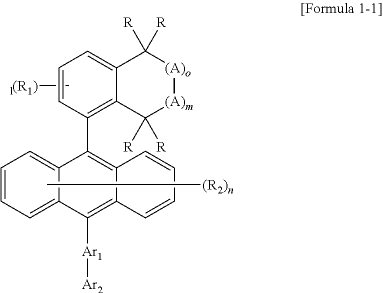
wherein R₁, R₂, Ar₁, Ar₂, A, R, l, m, n, and o are as defined in Formula 1; 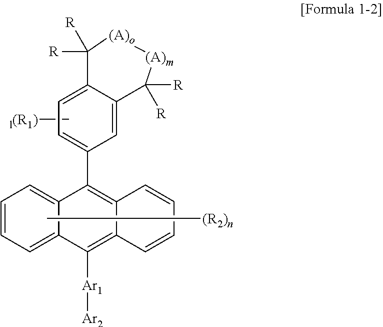
wherein R₁, R₂, Ar₁, Ar₂, A, R, l, m, n, and o are as defined in Formula 1.

3. The anthracene derivative according to claim 2, wherein the anthracene derivative of Formula 1-1 is represented by Formula 1-3: 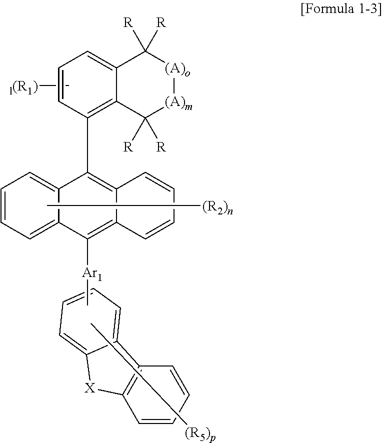
wherein X is O or S, the groups R₅ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in R₅ are optionally replaced by deuterium atoms and the adjacent R₅ groups are optionally linked to each other to form an alicyclic or aromatic monocyclic or polycyclic ring, p is an integer of 7, the groups R₅ are identical to or different from each other, and R₁, R₂, Ar₁, A, R, 1, m, n, and o are as defined in Formula 1; and the anthracene derivative of Formula 1-2 is represented by Formula 1-4: 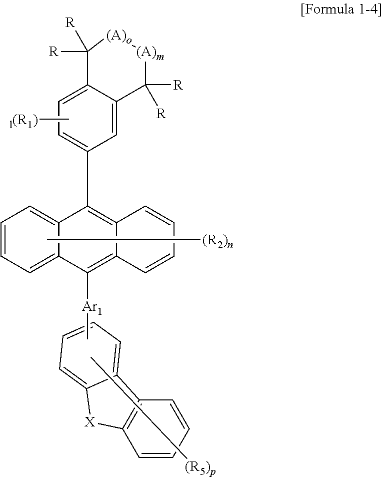
wherein X, R₅, and p are as defined in Formula 1-3 and R₁, R₂, Ar₁, A, R, 1, m, n, and o are as defined in Formula 1.

4. The anthracene derivative according to claim 1, wherein at least four of the groups R₂ in Formula 1 are deuterium.

5. The anthracene derivative according to claim 1, wherein at least one of the groups R₁ in Formula 1 is substituted or unsubstituted C₆-C₂₀ aryl.

6. The anthracene derivative according to claim 1, wherein the anthracene derivative represented by Formula 1 is selected from the following compounds:

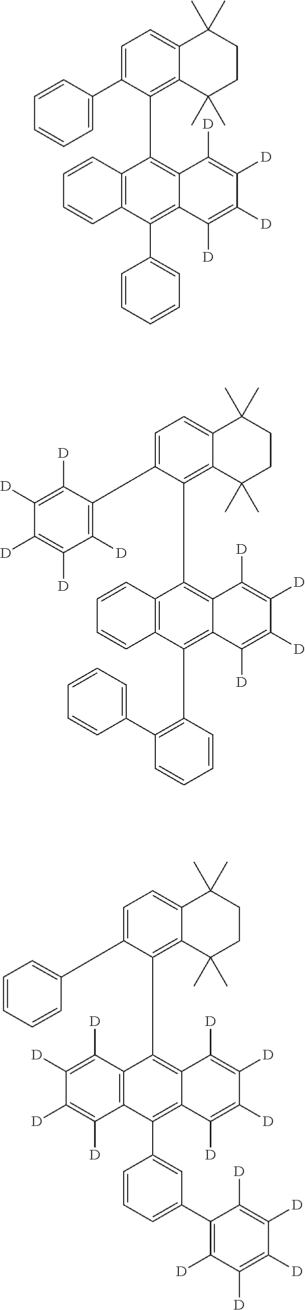 [SMILES: [2H]c1c([2H])c([2H])c2c(-c3c(-c4ccccc4)ccc4c3C(C)(C)CCC4(C)C)c3ccccc3c(-c3ccccc3)c2c1[2H] ; [2H]c1c([2H])c([2H])c(-c2ccc3c(c2-c2c4ccccc4c(-c4ccccc4-c4ccccc4)c4c([2H])c([2H])c([2H])c([2H])c24)C(C)(C)CCC3(C)C)c([2H])c1[2H] ; [2H]c1c([2H])c([2H])c(-c2cccc(-c3c4c([2H])c([2H])c([2H])c([2H])c4c(-c4c(-c5ccccc5)ccc5c4C(C)(C)CCC5(C)C)c4c([2H])c([2H])c([2H])c([2H])c34)c2)c([2H])c1[2H]]

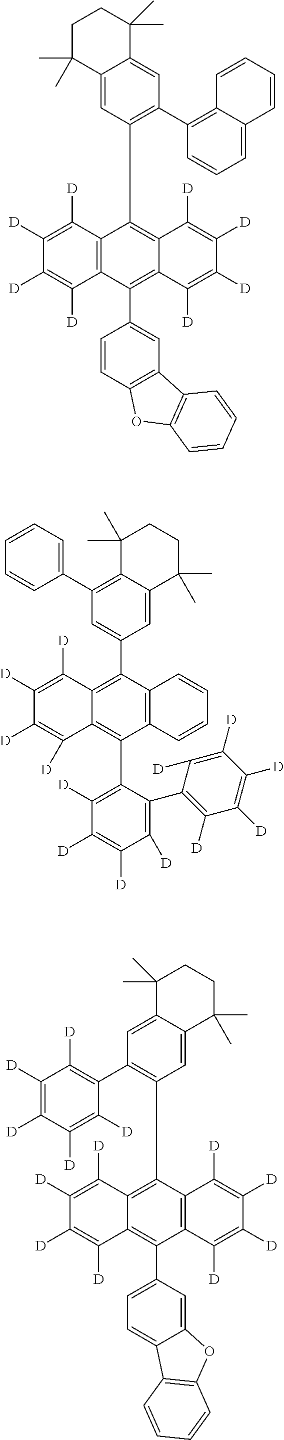 [SMILES: [2H]c1c([2H])c([2H])c2c(-c3cc4c(cc3-c3cccc5ccccc35)C(C)(C)CCC4(C)C)c3c([2H])c([2H])c([2H])c([2H])c3c(-c3ccc4oc5ccccc5c4c3)c2c1[2H] ; [2H]c1c([2H])c([2H])c(-c2c([2H])c([2H])c([2H])c([2H])c2-c2c3ccccc3c(-c3cc(-c4ccccc4)c4c(c3)C(C)(C)CCC4(C)C)c3c([2H])c([2H])c([2H])c([2H])c23)c([2H])c1[2H] ; [2H]c1c([2H])c([2H])c(-c2cc3c(cc2-c2c4c([2H])c([2H])c([2H])c([2H])c4c(-c4ccc5c(c4)oc4ccccc45)c4c([2H])c([2H])c([2H])c([2H])c24)C(C)(C)CCC3(C)C)c([2H])c1[2H]]

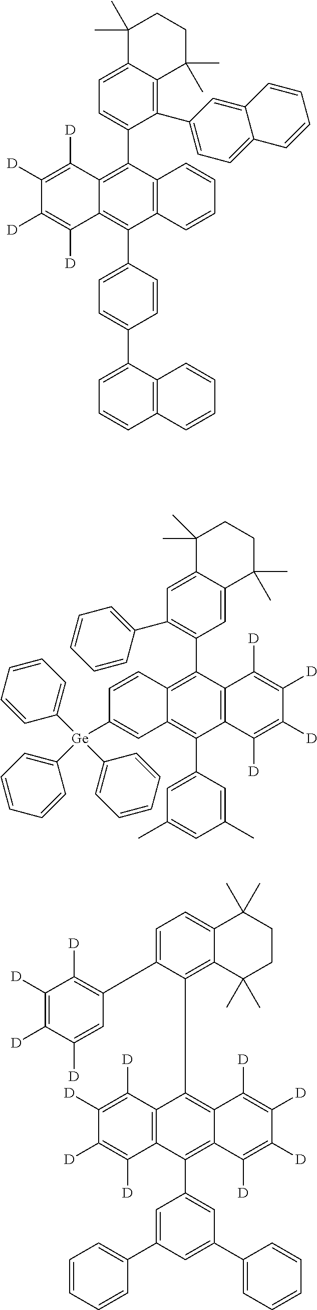

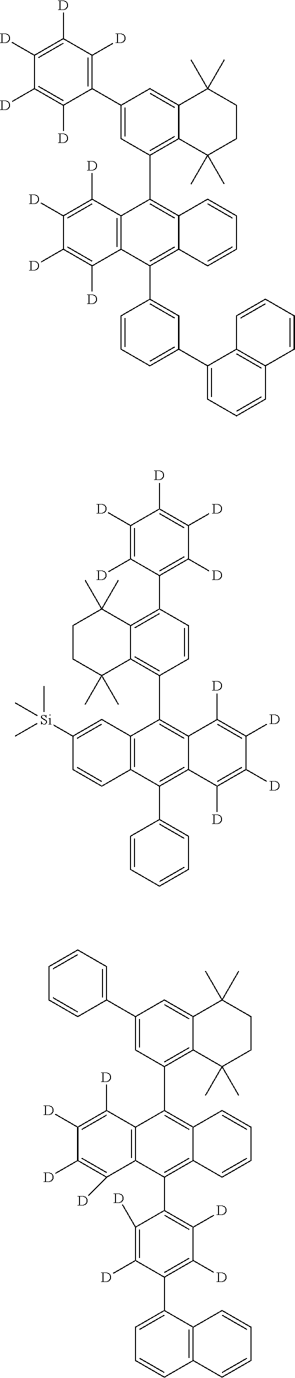 [SMILES: [2H]c1c([2H])c([2H])c(-c2cc(-c3c4ccccc4c(-c4cccc(-c5cccc6ccccc56)c4)c4c([2H])c([2H])c([2H])c([2H])c34)c3c(c2)C(C)(C)CCC3(C)C)c([2H])c1[2H] ; [2H]c1c([2H])c([2H])c(-c2ccc(-c3c4cc([Si](C)(C)C)ccc4c(-c4ccccc4)c4c([2H])c([2H])c([2H])c([2H])c34)c3c2C(C)(C)CCC3(C)C)c([2H])c1[2H] ; [2H]c1c([2H])c(-c2c3ccccc3c(-c3cc(-c4ccccc4)cc4c3C(C)(C)CCC4(C)C)c3c([2H])c([2H])c([2H])c([2H])c23)c([2H])c([2H])c1-c1cccc2ccccc12]

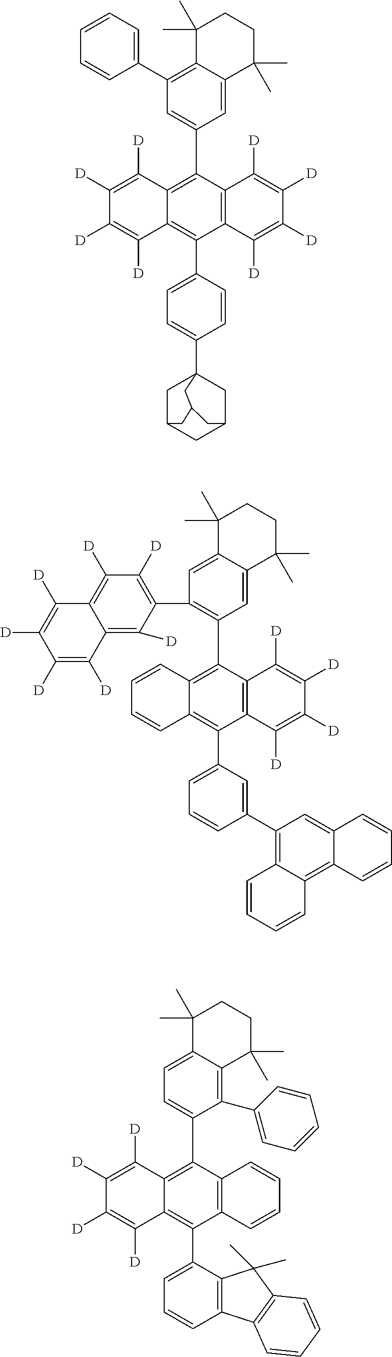 [SMILES: [2H]c1c([2H])c([2H])c2c(-c3cc(-c4ccccc4)c4c(c3)C(C)(C)CCC4(C)C)c3c([2H])c([2H])c([2H])c([2H])c3c(-c3ccc(C45CC6CC(CC(C6)C4)C5)cc3)c2c1[2H] ; [2H]c1c([2H])c([2H])c2c([2H])c(-c3cc4c(cc3-c3c5ccccc5c(-c5cccc(-c6cc7ccccc7c7ccccc67)c5)c5c([2H])c([2H])c([2H])c([2H])c35)C(C)(C)CCC4(C)C)c([2H])c([2H])c2c1[2H] ; [2H]c1c([2H])c([2H])c2c(-c3cccc4c3C(C)(C)c3ccccc3-4)c3ccccc3c(-c3ccc4c(c3-c3ccccc3)C(C)(C)CCC4(C)C)c2c1[2H]]

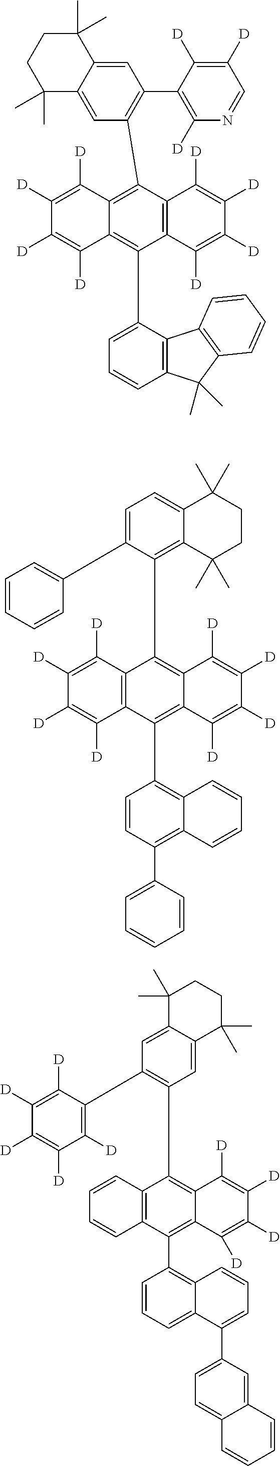

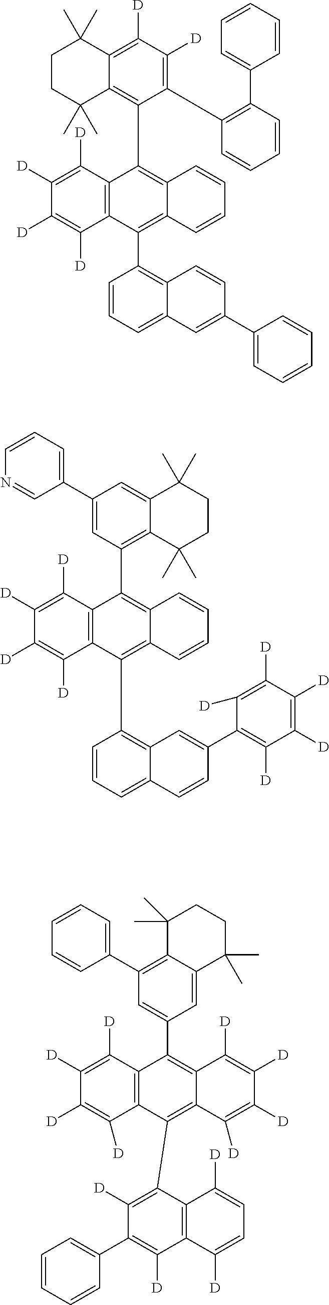 [SMILES: [2H]c1c([2H])c2c(c(-c3c4ccccc4c(-c4cccc5cc(-c6ccccc6)ccc45)c4c([2H])c([2H])c([2H])c([2H])c34)c1-c1ccccc1-c1ccccc1)C(C)(C)CCC2(C)C ; [2H]c1c([2H])c([2H])c(-c2ccc3cccc(-c4c5ccccc5c(-c5cc(-c6cccnc6)cc6c5C(C)(C)CCC6(C)C)c5c([2H])c([2H])c([2H])c([2H])c45)c3c2)c([2H])c1[2H] ; [2H]c1c([2H])c([2H])c2c(-c3c([2H])c(-c4ccccc4)c([2H])c4c([2H])ccc([2H])c34)c3c([2H])c([2H])c([2H])c([2H])c3c(-c3cc(-c4ccccc4)c4c(c3)C(C)(C)CCC4(C)C)c2c1[2H]]

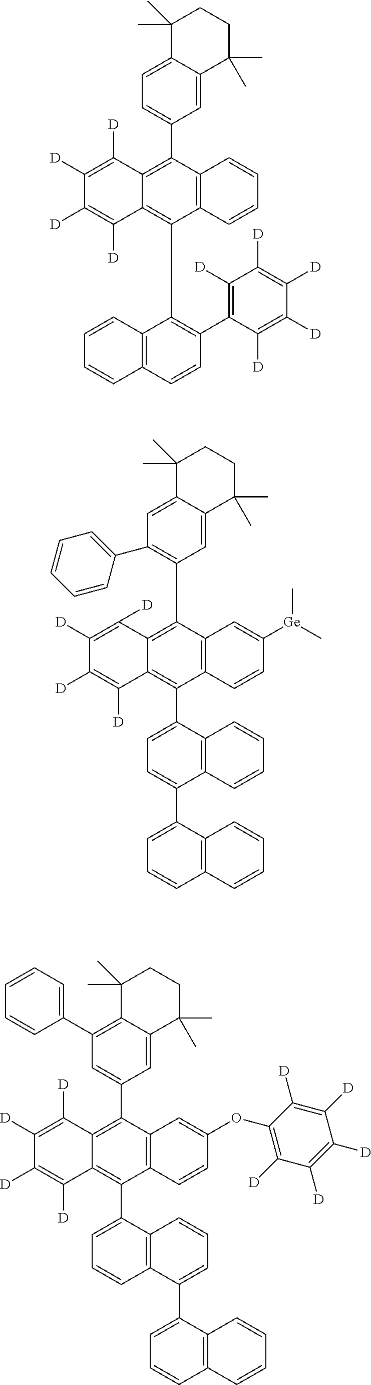

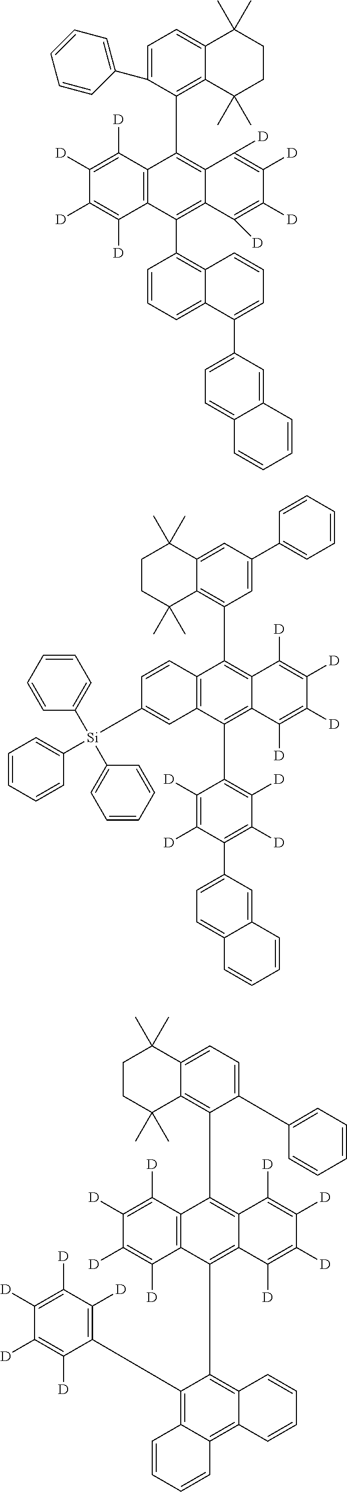

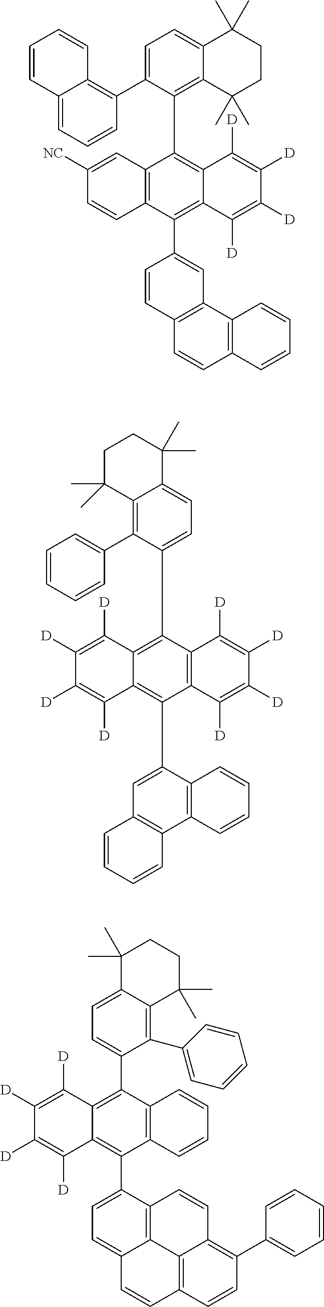 [SMILES: [2H]c1c([2H])c([2H])c2c(-c3c(-c4cccc5ccccc45)ccc4c3C(C)(C)CCC4(C)C)c3cc(C#N)ccc3c(-c3ccc4ccc5ccccc5c4c3)c2c1[2H] ; [2H]c1c([2H])c([2H])c2c(-c3cc4ccccc4c4ccccc34)c3c([2H])c([2H])c([2H])c([2H])c3c(-c3ccc4c(c3-c3ccccc3)C(C)(C)CCC4(C)C)c2c1[2H] ; [2H]c1c([2H])c([2H])c2c(-c3ccc4ccc5ccc(-c6ccccc6)c6ccc3c4c56)c3ccccc3c(-c3ccc4c(c3-c3ccccc3)C(C)(C)CCC4(C)C)c2c1[2H]]

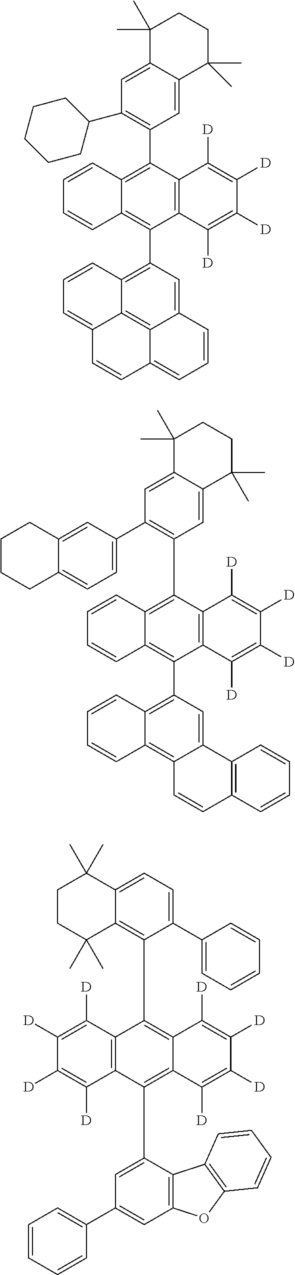

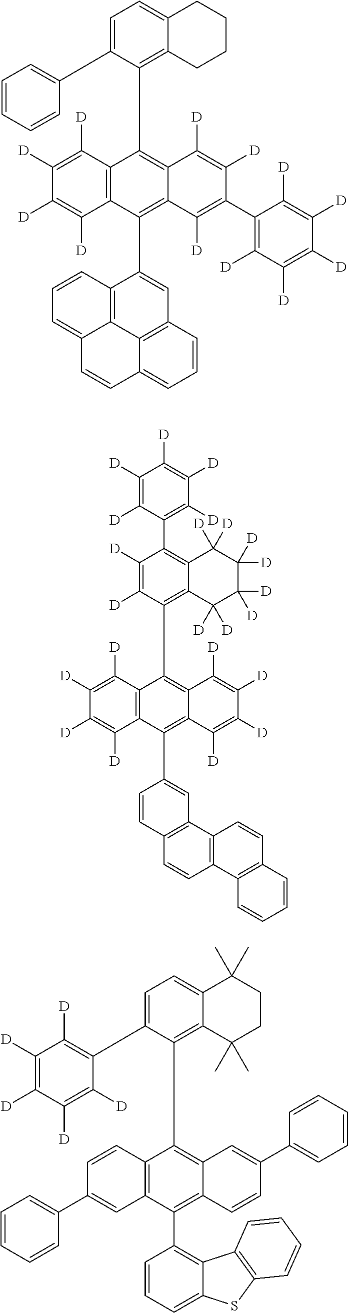

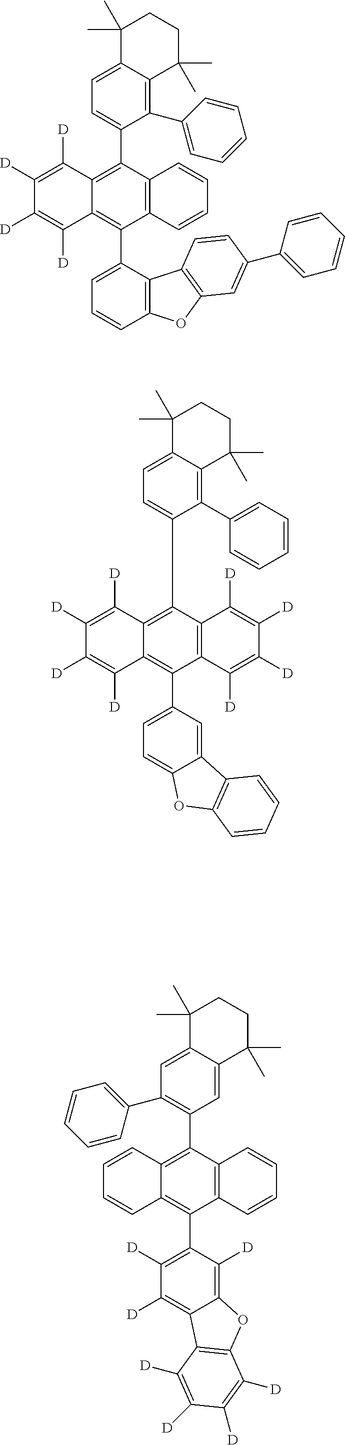 [SMILES: [2H]c1c([2H])c([2H])c2c(-c3cccc4oc5cc(-c6ccccc6)ccc5c34)c3ccccc3c(-c3ccc4c(c3-c3ccccc3)C(C)(C)CCC4(C)C)c2c1[2H] ; [2H]c1c([2H])c([2H])c2c(-c3ccc4c(c3-c3ccccc3)C(C)(C)CCC4(C)C)c3c([2H])c([2H])c([2H])c([2H])c3c(-c3ccc4oc5ccccc5c4c3)c2c1[2H] ; [2H]c1c([2H])c([2H])c2c(oc3c([2H])c(-c4c5ccccc5c(-c5cc6c(cc5-c5ccccc5)C(C)(C)CCC6(C)C)c5ccccc45)c([2H])c([2H])c32)c1[2H]]

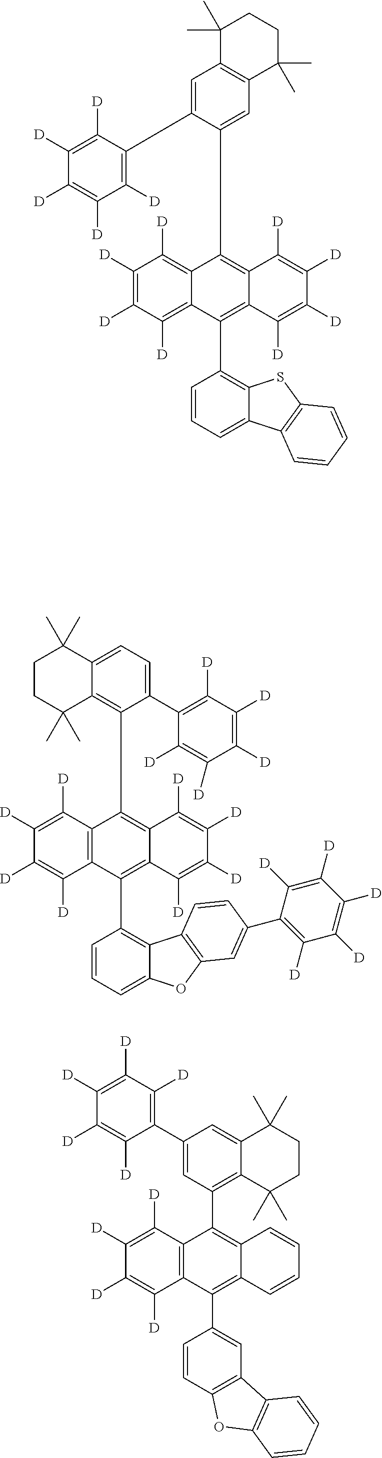 [SMILES: [2H]c1c([2H])c([2H])c(-c2cc3c(cc2-c2c4c([2H])c([2H])c([2H])c([2H])c4c(-c4cccc5c4sc4ccccc45)c4c([2H])c([2H])c([2H])c([2H])c24)C(C)(C)CCC3(C)C)c([2H])c1[2H] ; [2H]c1c([2H])c([2H])c(-c2ccc3c(c2)oc2cccc(-c4c5c([2H])c([2H])c([2H])c([2H])c5c(-c5c(-c6c([2H])c([2H])c([2H])c([2H])c6[2H])ccc6c5C(C)(C)CCC6(C)C)c5c([2H])c([2H])c([2H])c([2H])c45)c23)c([2H])c1[2H] ; [2H]c1c([2H])c([2H])c(-c2cc(-c3c4ccccc4c(-c4ccc5oc6ccccc6c5c4)c4c([2H])c([2H])c([2H])c([2H])c34)c3c(c2)C(C)(C)CCC3(C)C)c([2H])c1[2H]]

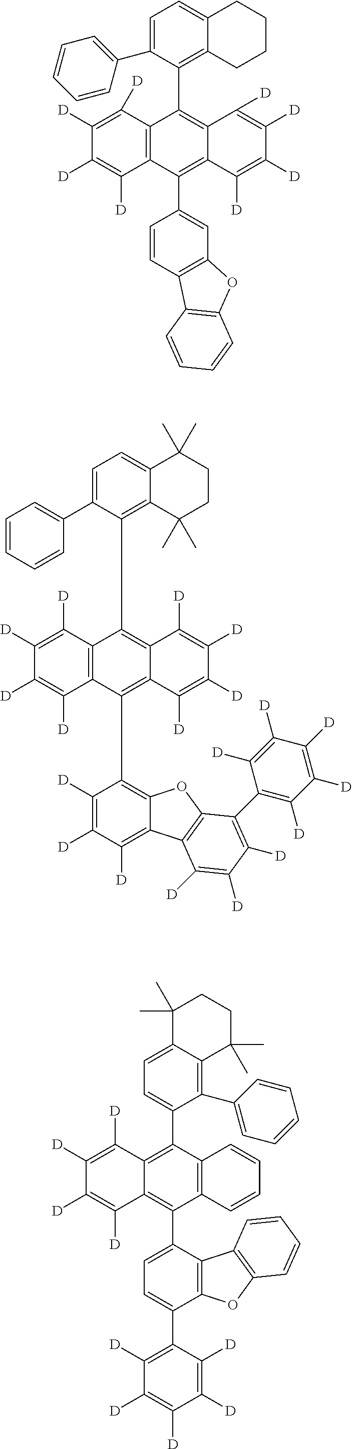 [SMILES: [2H]c1c([2H])c([2H])c2c(-c3c(-c4ccccc4)ccc4c3CCCC4)c3c([2H])c([2H])c([2H])c([2H])c3c(-c3ccc4c(c3)oc3ccccc34)c2c1[2H] ; [2H]c1c([2H])c([2H])c(-c2c([2H])c([2H])c([2H])c3c2oc2c(-c4c5c([2H])c([2H])c([2H])c([2H])c5c(-c5c(-c6ccccc6)ccc6c5C(C)(C)CCC6(C)C)c5c([2H])c([2H])c([2H])c([2H])c45)c([2H])c([2H])c([2H])c23)c([2H])c1[2H] ; [2H]c1c([2H])c([2H])c(-c2ccc(-c3c4ccccc4c(-c4ccc5c(c4-c4ccccc4)C(C)(C)CCC5(C)C)c4c([2H])c([2H])c([2H])c([2H])c34)c3c2oc2ccccc23)c([2H])c1[2H]]

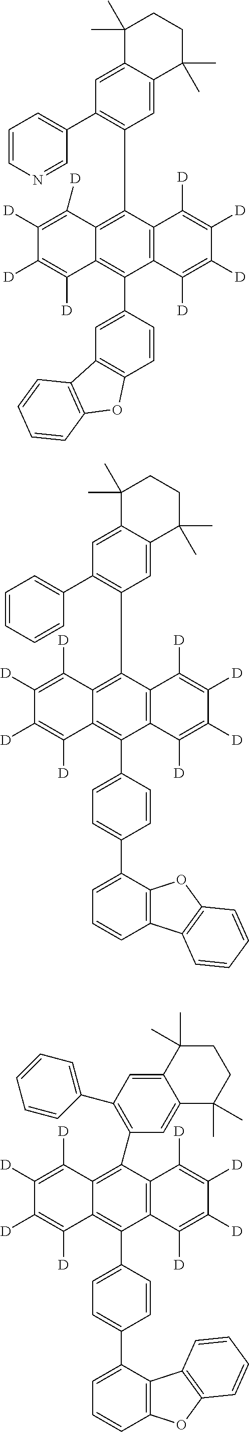

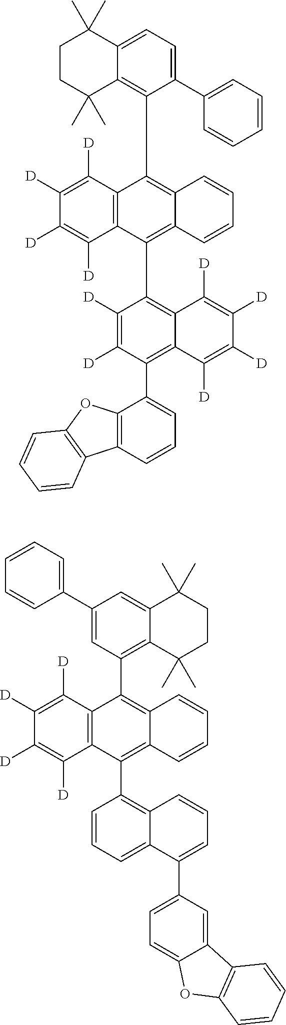 [SMILES: [2H]c1c([2H])c([2H])c2c(-c3c4ccccc4c(-c4c(-c5ccccc5)ccc5c4C(C)(C)CCC5(C)C)c4c([2H])c([2H])c([2H])c([2H])c34)c([2H])c([2H])c(-c3cccc4c3oc3ccccc34)c2c1[2H] ; [2H]c1c([2H])c([2H])c2c(-c3cccc4c(-c5ccc6oc7ccccc7c6c5)cccc34)c3ccccc3c(-c3cc(-c4ccccc4)cc4c3C(C)(C)CCC4(C)C)c2c1[2H]]

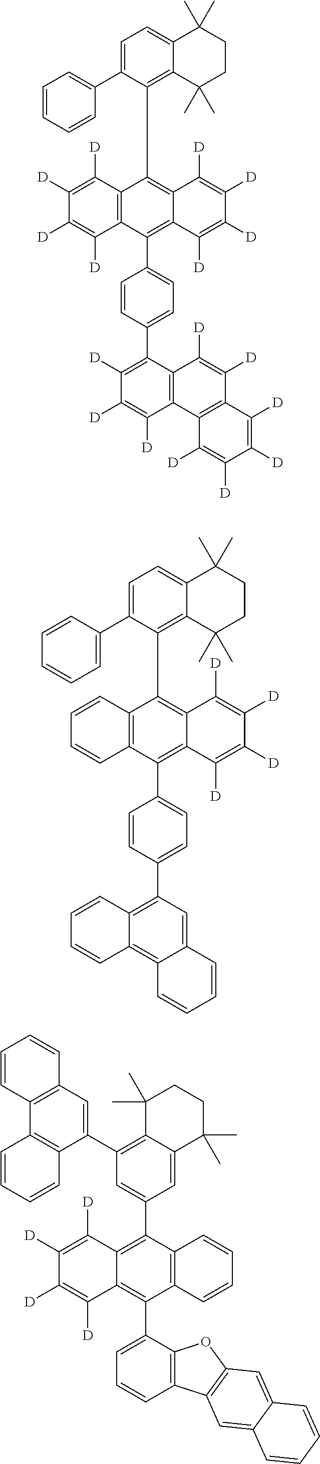

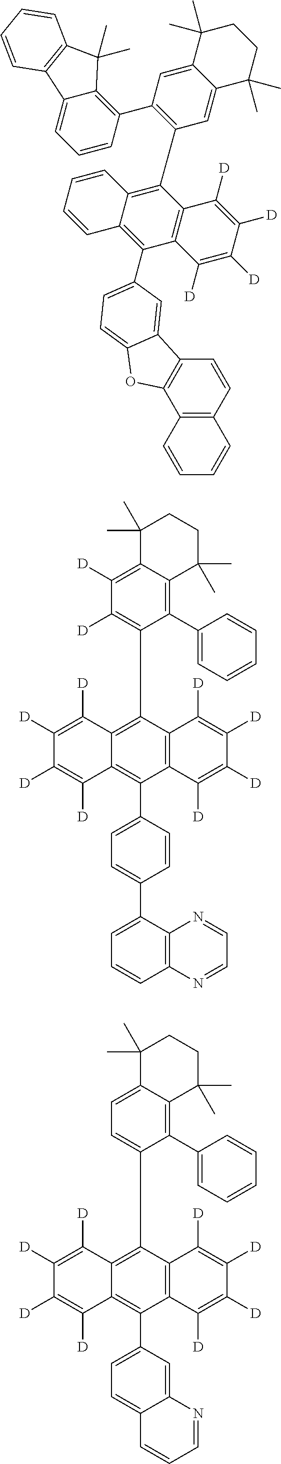

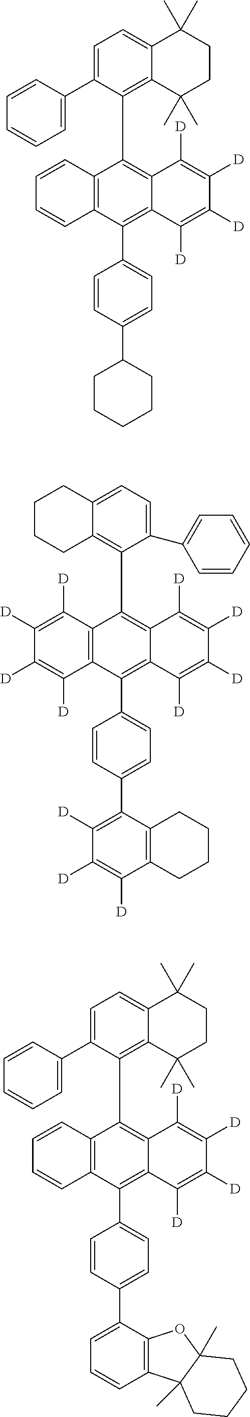 [SMILES: [2H]c1c([2H])c([2H])c2c(-c3c(-c4ccccc4)ccc4c3C(C)(C)CCC4(C)C)c3ccccc3c(-c3ccc(C4CCCCC4)cc3)c2c1[2H] ; [2H]c1c([2H])c2c(c(-c3ccc(-c4c5c([2H])c([2H])c([2H])c([2H])c5c(-c5c(-c6ccccc6)ccc6c5CCCC6)c5c([2H])c([2H])c([2H])c([2H])c45)cc3)c1[2H])CCCC2 ; [2H]c1c([2H])c([2H])c2c(-c3c(-c4ccccc4)ccc4c3C(C)(C)CCC4(C)C)c3ccccc3c(-c3ccc(-c4cccc5c4OC4(C)CCCCC54C)cc3)c2c1[2H]]

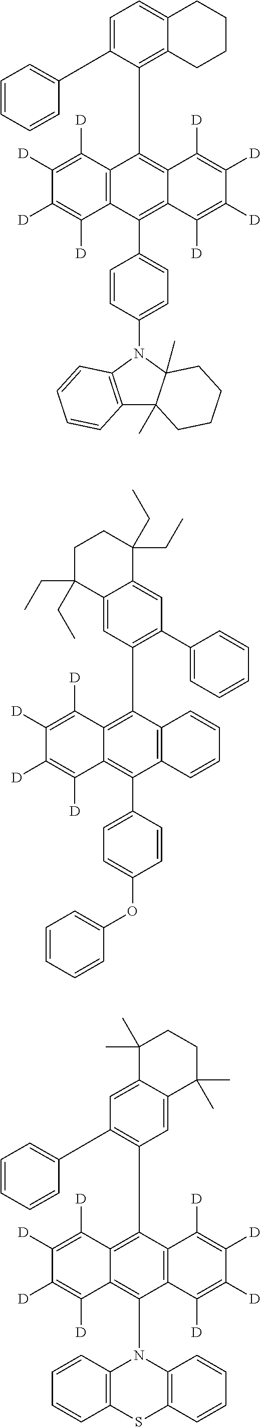 [SMILES: [2H]c1c([2H])c([2H])c2c(-c3c(-c4ccccc4)ccc4c3CCCC4)c3c([2H])c([2H])c([2H])c([2H])c3c(-c3ccc(N4c5ccccc5C5(C)CCCCC45C)cc3)c2c1[2H] ; [2H]c1c([2H])c([2H])c2c(-c3cc4c(cc3-c3ccccc3)C(CC)(CC)CCC4(CC)CC)c3ccccc3c(-c3ccc(Oc4ccccc4)cc3)c2c1[2H] ; [2H]c1c([2H])c([2H])c2c(N3c4ccccc4Sc4ccccc43)c3c([2H])c([2H])c([2H])c([2H])c3c(-c3cc4c(cc3-c3ccccc3)C(C)(C)CCC4(C)C)c2c1[2H]]

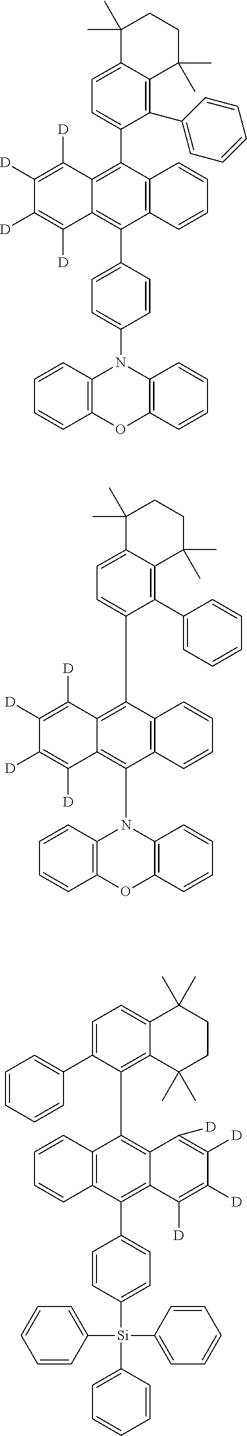 [SMILES: [2H]c1c([2H])c([2H])c2c(-c3ccc4c(c3-c3ccccc3)C(C)(C)CCC4(C)C)c3ccccc3c(-c3ccc(N4c5ccccc5Oc5ccccc54)cc3)c2c1[2H] ; [2H]c1c([2H])c([2H])c2c(N3c4ccccc4Oc4ccccc43)c3ccccc3c(-c3ccc4c(c3-c3ccccc3)C(C)(C)CCC4(C)C)c2c1[2H] ; [2H]c1c([2H])c([2H])c2c(-c3c(-c4ccccc4)ccc4c3C(C)(C)CCC4(C)C)c3ccccc3c(-c3ccc([Si](c4ccccc4)(c4ccccc4)c4ccccc4)cc3)c2c1[2H]]

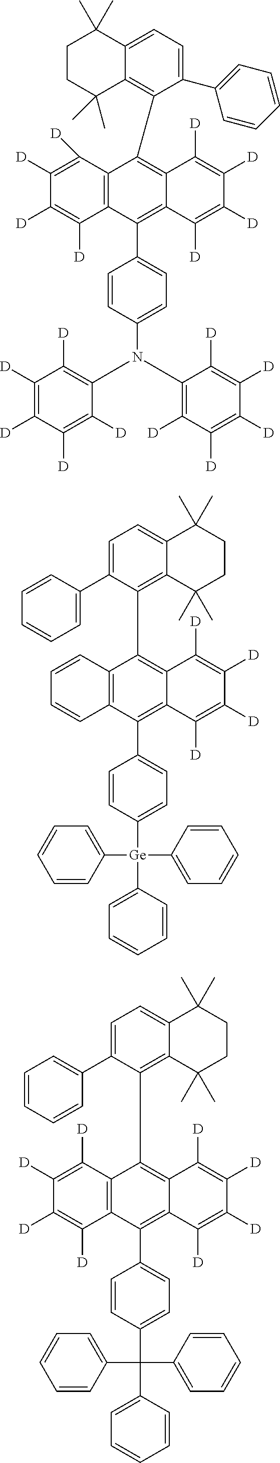

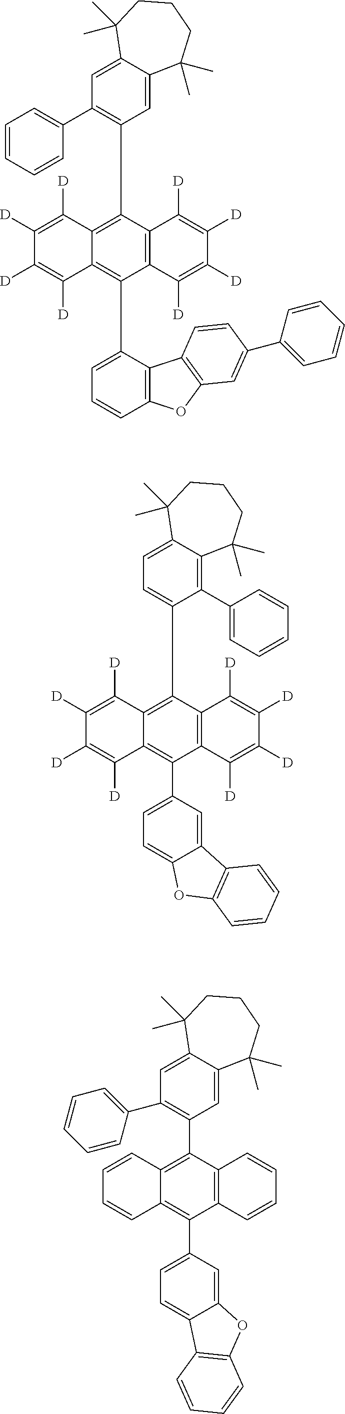 [SMILES: [2H]c1c([2H])c([2H])c2c(-c3cccc4oc5cc(-c6ccccc6)ccc5c34)c3c([2H])c([2H])c([2H])c([2H])c3c(-c3cc4c(cc3-c3ccccc3)C(C)(C)CCCC4(C)C)c2c1[2H] ; [2H]c1c([2H])c([2H])c2c(-c3ccc4c(c3-c3ccccc3)C(C)(C)CCCC4(C)C)c3c([2H])c([2H])c([2H])c([2H])c3c(-c3ccc4oc5ccccc5c4c3)c2c1[2H] ; CC1(C)CCCC(C)(C)c2cc(-c3c4ccccc4c(-c4ccc5c(c4)oc4ccccc45)c4ccccc34)c(-c3ccccc3)cc21]

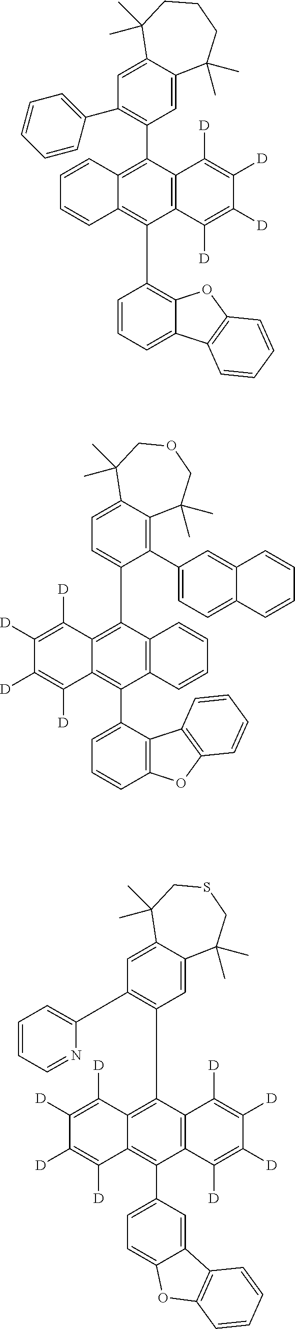 [SMILES: [2H]c1c([2H])c([2H])c2c(-c3cccc4c3oc3ccccc34)c3ccccc3c(-c3cc4c(cc3-c3ccccc3)C(C)(C)CCCC4(C)C)c2c1[2H] ; [2H]c1c([2H])c([2H])c2c(-c3cccc4oc5ccccc5c34)c3ccccc3c(-c3ccc4c(c3-c3ccc5ccccc5c3)C(C)(C)COCC4(C)C)c2c1[2H] ; [2H]c1c([2H])c([2H])c2c(-c3cc4c(cc3-c3ccccn3)C(C)(C)CSCC4(C)C)c3c([2H])c([2H])c([2H])c([2H])c3c(-c3ccc4oc5ccccc5c4c3)c2c1[2H]]

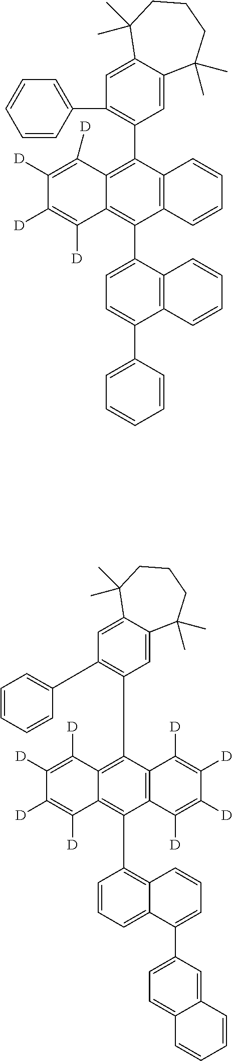 [SMILES: [2H]c1c([2H])c([2H])c2c(-c3ccc(-c4ccccc4)c4ccccc34)c3ccccc3c(-c3cc4c(cc3-c3ccccc3)C(C)(C)CCCC4(C)C)c2c1[2H] ; [2H]c1c([2H])c([2H])c2c(-c3cccc4c(-c5ccc6ccccc6c5)cccc34)c3c([2H])c([2H])c([2H])c([2H])c3c(-c3cc4c(cc3-c3ccccc3)C(C)(C)CCCC4(C)C)c2c1[2H]]

7. An organic light emitting device comprising a first electrode, a second electrode, and one or more organic layers interposed between the first and second electrodes wherein one of the organic layers is a light emitting layer, the light emitting layer comprises at least one host and at least one dopant, the host is an anthracene derivative represented by Formula 1: 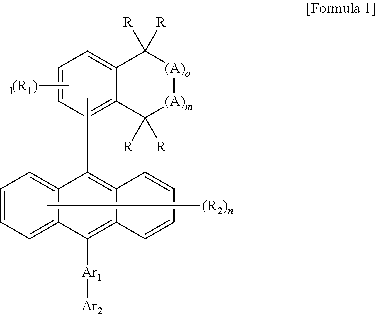
wherein the moieties A are identical to or different from each other and are each independently selected from O, S, and CR₃R₄, R, R₃, and R₄ are identical to or different from each other and are each independently selected from hydrogen, deuterium, and substituted or unsubstituted C₁-C₃₀ alkyl, with the proviso that adjacent ones of R, R₃, and R₄ are optionally linked to each other or each of R, R₃, and R₄ is optionally linked to an adjacent substituent to form an alicyclic or aromatic monocyclic or polycyclic ring, the groups R₁ are identical to or different from each other and are each independently selected from hydrogen, deuterium, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted C₃-C₅₀ mixed aliphatic-aromatic cyclic groups, the groups R₂ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, Ar₁ represents a single bond or is substituted or unsubstituted C₆-C₅₀ arylene or substituted or unsubstituted C₂-C₅₀ heteroarylene, Ar₂ is selected from substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted C₃-C₅₀ mixed aliphatic-aromatic cyclic groups, 1 is an integer from 1 to 3, with the proviso that when 1 is 2 or more, the groups Ri are identical to or different from each other, each of o and m is independently an integer of 1 or 2, provided that when o or m is 2 or more, the moieties A are identical to or different from each other, n is an integer of 8, and the groups R₂ are identical to or different from each other; and the dopant is a polycyclic compound represented by Formula 2: 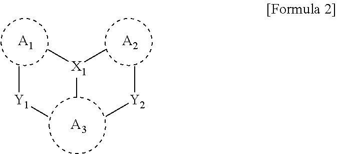
wherein X₁ is B, Y₁ and Y₂ are identical to or different from each other and are each independently selected from NR₁₁, O, S, CR₁₂R₁₃, SiR₁₄R₁₅ or GeR₁₆R₁₇, A₁ to A₃ are identical to or different from each other and are each independently selected from substituted or unsubstituted C₆-C₅₀ aromatic hydrocarbon rings, substituted or unsubstituted C₃-C₅₀ aliphatic hydrocarbon rings, substituted or unsubstituted C₂-C₅₀ aromatic heterocyclic rings, substituted or unsubstituted C₂-C₅₀ aliphatic heterocyclic rings, and substituted or unsubstituted C₃-C₅₀ mixed aliphatic-aromatic cyclic group, R₁₁ to R₁₇ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, with the proviso that each of R₁₁ to R₁₇ is optionally linked to one or more of the rings A₁ to A₃ to form an alicyclic or aromatic monocyclic or polycyclic ring, R₁₂ and R₁₃ are optionally linked to each other to form an alicyclic or aromatic monocyclic or polycyclic ring, R₁₄ and R₁₅ are optionally linked to each other to form an alicyclic or aromatic monocyclic or polycyclic ring, and R 16 and R 17 are optionally linked to each other to form an alicyclic or aromatic monocyclic or polycyclic ring, the “substituted” in the definitions of Y₁, Y₂, and A1 to A3 in Formula 2 indicating substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, C₃-C₃₀ mixed aliphatic-aromatic cyclic groups, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof, the “unsubstituted” in the same definition indicating having no substituent, and one or more hydrogen atoms in each of the substituents are optionally replaced by deuterium atoms.

8. The organic light emitting device according to claim 7, wherein the compound represented by Formula 2 is a polycyclic compound represented by Formula 3 or 4: 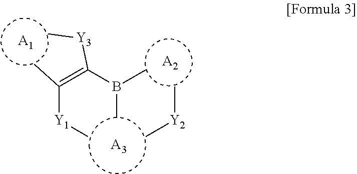
wherein Y₃ is O, S or NR₁₈, R₁₅ is as defined for R₁₁ to R₁₇ in Formula 2, and Y₁, Y₂, and A1 to A3 are as defined in Formula 2; 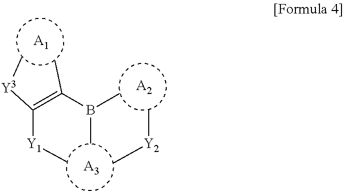
wherein Y₃ is O, S or NR18, R₁₈ is as defined for R₁ to R₁₇ in Formula 2, and Y₁, Y₂, and A1 to A3 are as defined in Formula 2.

9. The organic light emitting device according to claim 7, wherein the compound represented by Formula 2 is a polycyclic compound represented by one of Formulas 3-1, 3-2, 3-3, 4-1, 4-2, and 4-3: 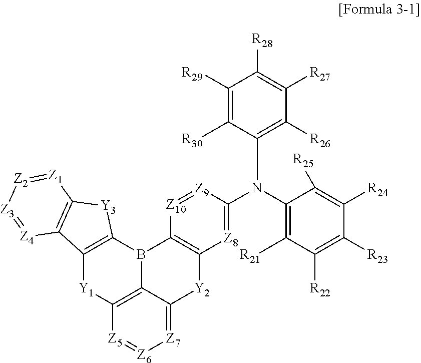
wherein Z₁ to Zio are identical to or different from each other and are each independently CR₃₁ or N, provided that when two or more of Z₁ to Z₁₀ are CR₃₁, the moieties CR₃₁ are identical to or different from each other and adjacent ones of the groups R₃₁ are optionally linked to each other to form an alicyclic or aromatic monocyclic or polycyclic ring, Y₃ is O, S or NR₁₈, R₁₈ is as defined for R₁₁ to R₁₇ in Formula 2, the groups R₃₁ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, R₂₁ to R₃₀ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in each of the substituents of R₂₁ to R₃₁ are optionally replaced by deuterium atoms, adjacent ones of the substituents of R₂₁ to R₃₀ are optionally linked to each other to form an alicyclic or aromatic monocyclic or polycyclic ring, and the groups R₃₁ are optionally linked to each other to form an alicyclic or aromatic monocyclic or polycyclic ring, provided that when Z₈ and Z₉ are CR₃₁, each of R₂₁, R₂₅, R₂₆, and R₃₀ adjacent to Z₈ and Z₉ is optionally bonded to R₃₁ to form an alicyclic or aromatic monocyclic or polycyclic ring, Y₁ and Y₂ are identical to or different from each other and are each independently as defined in Formula 2 or are each independently a linker represented by Structural Formula A: 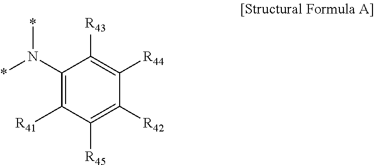
wherein R₄₁ to R₄₅ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in each of the substituents of R₄₁ to R₄₅ are optionally replaced by deuterium atoms and each of R₄₁ to R₄₅ is optionally linked to one or more adjacent substituents to form an alicyclic or aromatic monocyclic or polycyclic ring; 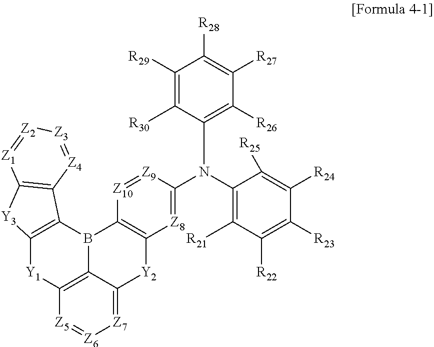
wherein Z₁ to Z₁₀, Y₁ to Y₃, and R₂₁ to R₃₀ are as defined in Formula 3-1; 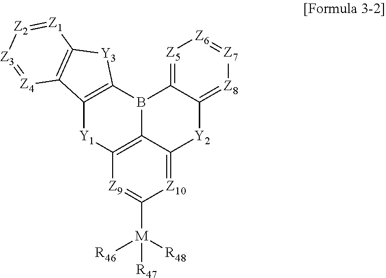
wherein Z₁ to Z₁₀ are identical to or different from each other and are each independently CR₃₁ or N, provided that when two or more of Z₁ to Z₁₀ are CR₃₁, the moieties CR₃₁ are identical to or different from each other and when two or more of Z₁ to Z₈ are CR₃₁, adjacent ones of the groups R₃₁ are optionally linked to each other to form an alicyclic or aromatic monocyclic or polycyclic ring, Y₃ is O, S or NR₁₈, R₁₈ is as defined for R₁₁ to R₁₇ in Formula 2, M is Si or Ge, the groups R₃₁ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, R₄₆ to R₄₈ are identical to or different from each other and are each independently selected from hydrogen, deuterium, substituted or unsubstituted C₁-C₂₄ alkyl, substituted or unsubstituted C₂-C₂₄ alkynyl, substituted or unsubstituted C₂-C₂₄ alkenyl, substituted or unsubstituted C₆-C₃₀ aryl, substituted or unsubstituted C₃-C₂₄ cycloalkyl, substituted or unsubstituted C₂-C₂₄ heterocycloalkyl, substituted or unsubstituted C₂-C₃₀ heteroaryl, substituted or unsubstituted C₃-C₃₀ mixed aliphatic-aromatic cyclic groups, substituted or unsubstituted C₁-C₂₄ alkoxy, substituted or unsubstituted C₆-C₂₄ aryloxy, substituted or unsubstituted C₁-C₂₄ alkylthioxy, substituted or unsubstituted C₅-C₂₄ arylthioxy, substituted or unsubstituted C₁-C₃₀ amine, substituted or unsubstituted C₁-C₃₀ silyl, substituted or unsubstituted C₁-C₃₀ germanium, nitro, cyano, and halogen, with the proviso that one or more hydrogen atoms in each of the substituents of R₃₁ and R₄₆ to R₄₈ are optionally replaced by deuterium atoms and the substituents of R₄₆ to R₄₈ are optionally linked to each other to form an alicyclic or aromatic monocyclic or polycyclic ring: 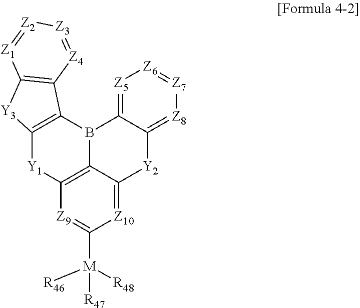
wherein Z₁ to Z₁₀, Y₁ to Y₃, M, and R₄₆ to R₄₈ are as defined in Formula 3-2; 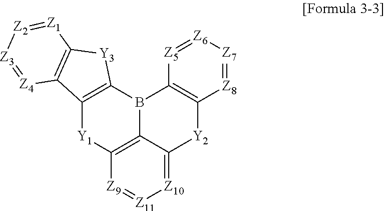
wherein Z₁ to Z₁₁ are identical to or different from each other and are each independently CR₃₁ or N, provided that when two or more of Z₁ to Z₁₁ are CR₃₁, the moieties CR₃₁ are identical to or different from each other and adjacent ones of the groups R₃₁ are optionally linked to each other to form an alicyclic or aromatic monocyclic or polycyclic ring, Y₃ is O, S or NR₁₈, R₁₈ is as defined for R₁ to R₁₇ in Formula 2, the groups R₃₁ are identical to or different from each other and are each independently selected from hydrogen, deuterium, substituted or unsubstituted C₁-C₃₀ alkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted C₃-C₅₀ mixed aliphatic-aromatic cyclic groups, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano, and halogen, with the proviso that one or more hydrogen atoms in each of the substituents of the groups R₃₁ are optionally replaced by deuterium atoms, Y₁ and Y₂ are identical to or different from each other and are each independently as defined in Formula 2 or are each independently a linker represented by Structural Formula B: 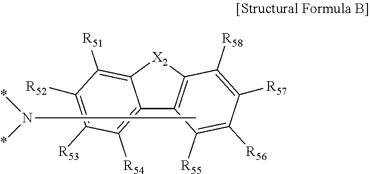
wherein X₂ is O or S, R₅₁ to R₅₈ are identical to or different from each other and are each independently selected from hydrogen, deuterium, C₁-C₂₄ alkyl, C₂-C₂₄ alkynyl, C₂-C₂₄ alkenyl, C₆-C₃₀ aryl, C₃-C₂₄ cycloalkyl, C₂-C₂₄ heterocycloalkyl, C₂-C₃₀ heteroaryl, C₃-C₃₀ mixed aliphatic-aromatic cyclic groups, C₇-C₃₀ arylalkyl, C₇-C₃₀ alkylaryl, C₃-C₃₀ heteroarylalkyl, C₃-C₃₀ alkylheteroaryl, C₁-C₂₄ alkoxy, C₆-C₂₄ aryloxy, C₁-C₂₄ alkylthioxy, C₅-C₂₄ arylthioxy, C₁-C₃₀ amine, C₁-C₃₀ silyl, C₁-C₃₀ germanium, nitro, cyano, and halogen, with the proviso that one or more hydrogen atoms in each of the substituents of R₅₁ to R₅₈ are optionally replaced by deuterium atoms, one of R₅₁ to R₅₈ forms a single bond with the nitrogen atom, and each of the others is optionally linked to one or more adjacent substituents to form an alicyclic or aromatic monocyclic or polycyclic ring; and 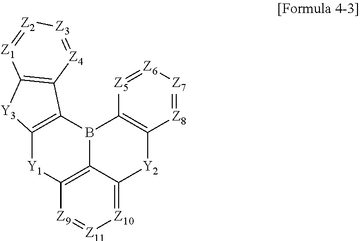
wherein Z₁ to Z₁₁ and Y₁ to Y₃ are as defined in Formula 3-3, the “substituted” in the definitions of Z₁ to Zio, Y₁ to Y₃, and R₂₁ to R₃₀ in Formula 3-1, Z₁ to Z₁₀, Y₁ to Y₃, and R₄₆ to R₄₈ in Formula 3-2, Z₁ to Z₁₁ and Y₁ to Y₃ in Formula 3-3, Z₁ to Z₁₀, Y₁ to Y₃, and R₂₁ to R₃₀ in Formula 4-1, Z₁ to Z₁₀, Y₁ to Y₃, and R₄₆ to R₄₈ in Formula 4-2, and Z₁ to Z₁₁ and Y₁ to Y₃ in Formula 4-3 indicating substitution with one or more substituents selected from deuterium, C₁-C₂₄ alkyl, C₁-C₂₄ haloalkyl, C₃-C₂₄ cycloalkyl, C₂-C₂₄ alkenyl, C₂-C₂₄ alkynyl, C₁-C₂₄ heteroalkyl, C₂-C₂₄ heterocycloalkyl, C₆-C₃₀ aryl, C₇-C₃₀ arylalkyl, C₇-C₃₀ alkylaryl, C₂-C₃₀ heteroaryl, C₃-C₃₀ heteroarylalkyl, C₃-C₃₀ alkylheteroaryl, C₃-C₃₀ mixed aliphatic-aromatic cyclic groups, C₁-C₂₄ alkoxy, C₆-C₂₄ aryloxy, C₆-C₂₄ arylthionyl, C₁-C₄₀ amine, C₁-C₄₀ silyl, C₁-C₄₀ germanium, cyano, halogen, hydroxyl, and nitro, or a combination thereof, the “unsubstituted” in the same definition indicating having no substituent, and one or more hydrogen atoms in each of the substituents being optionally replaced by deuterium atoms.

10. The organic light emitting device according to claim 7, wherein the polycyclic compound represented by Formula 2 is selected from the following compounds:

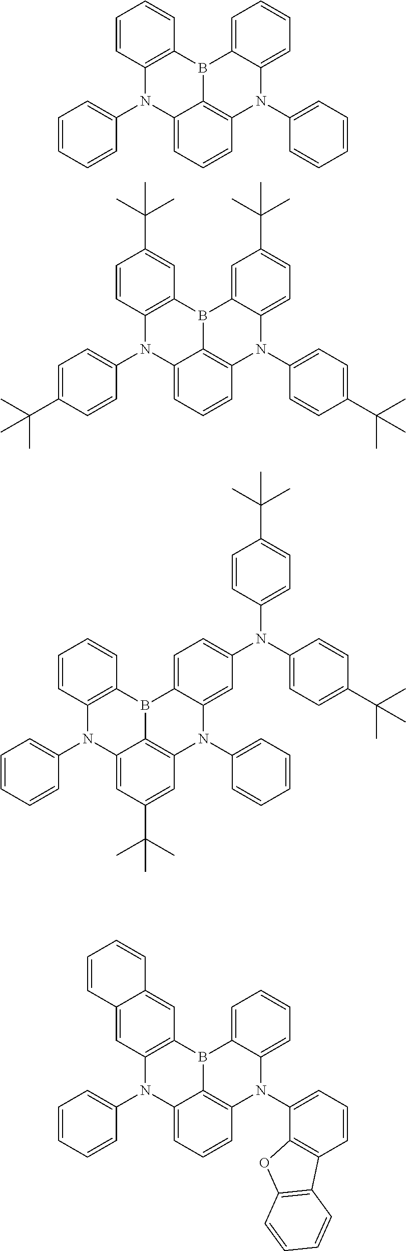

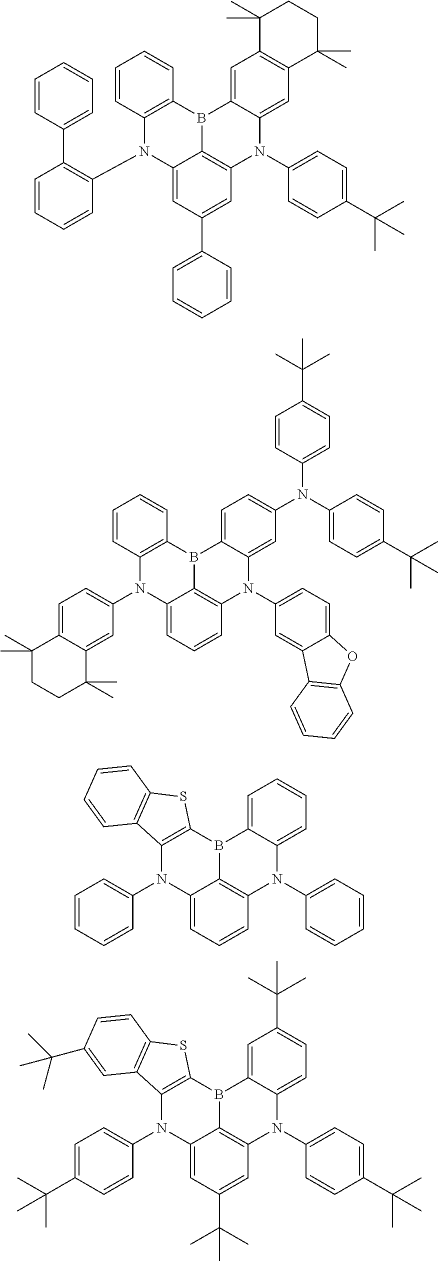

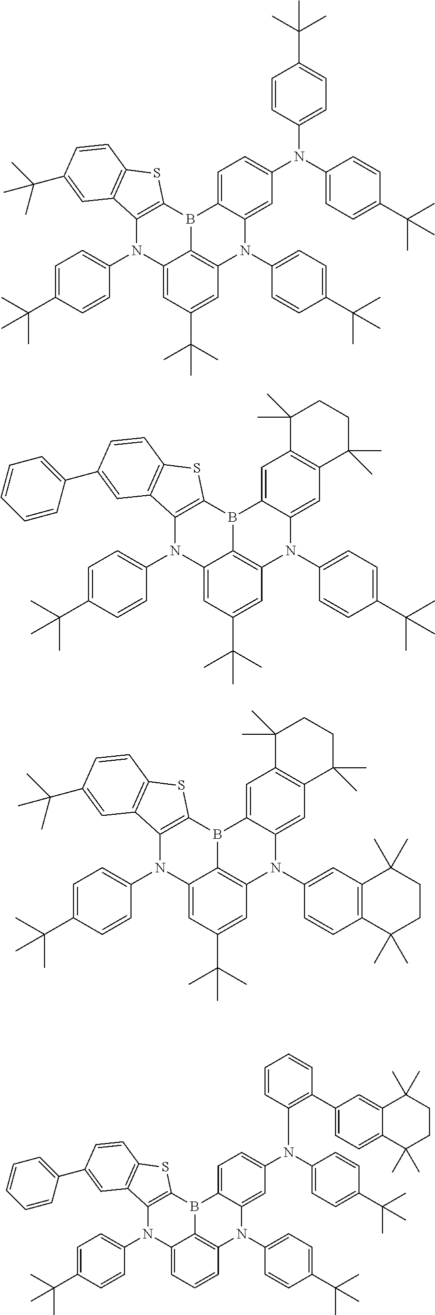

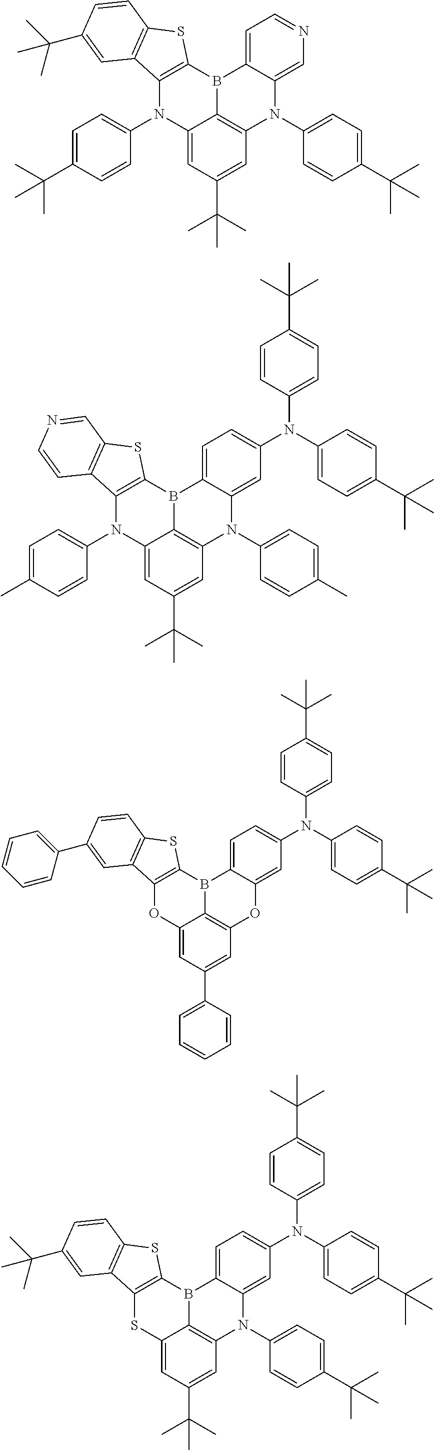

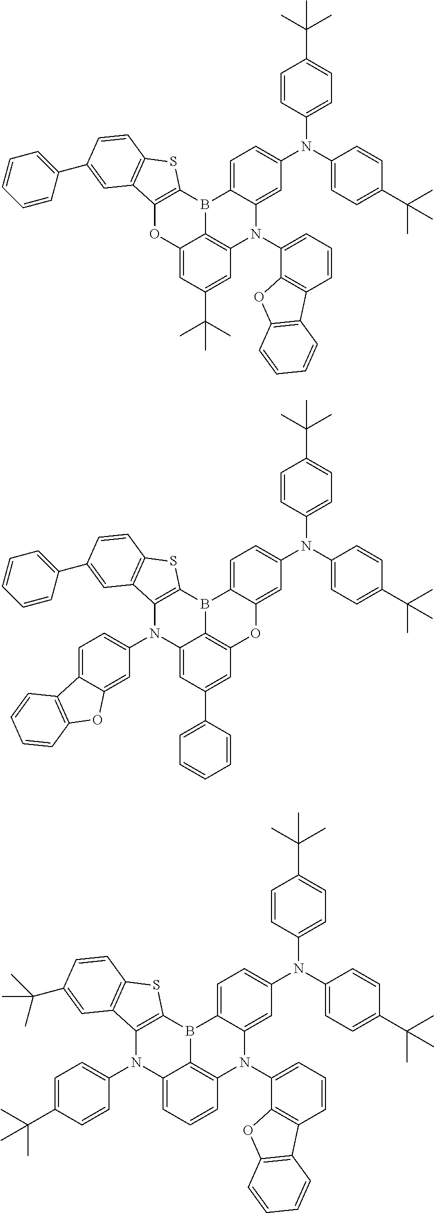

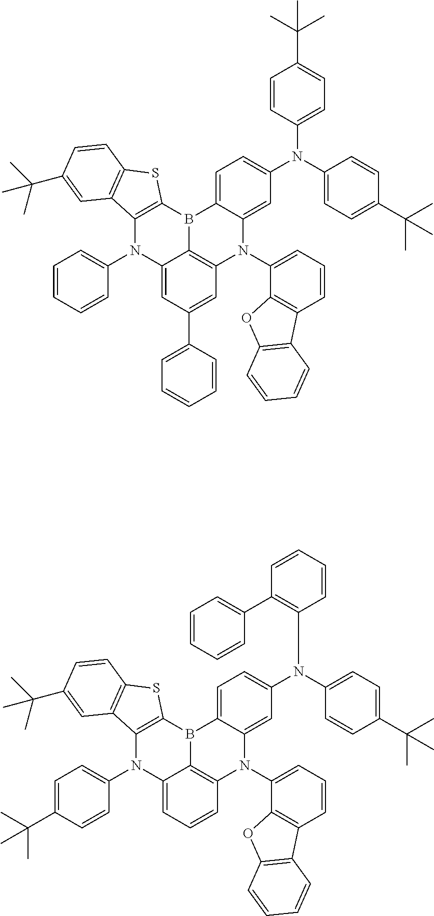

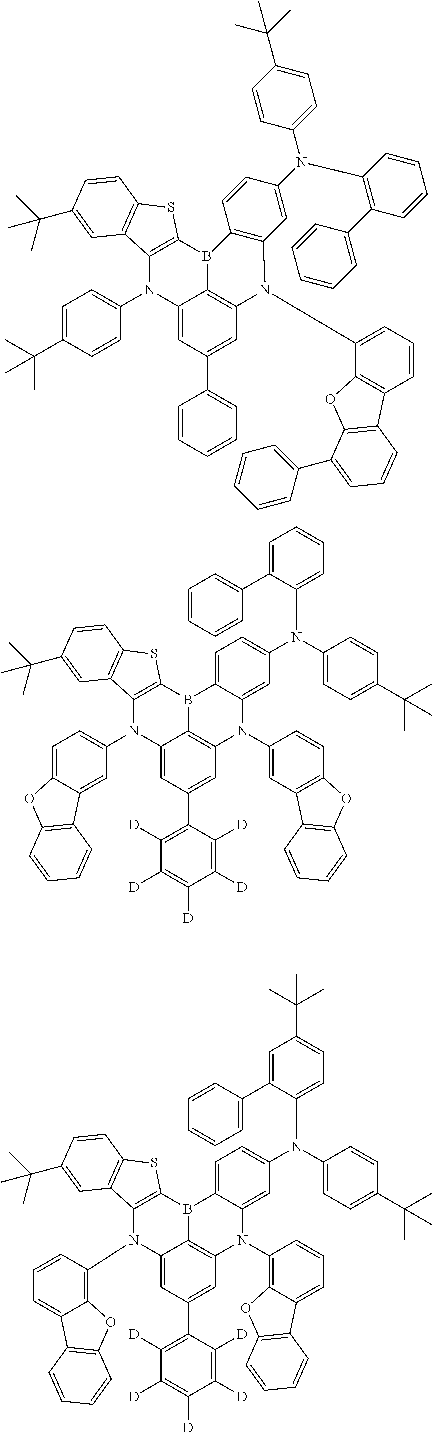

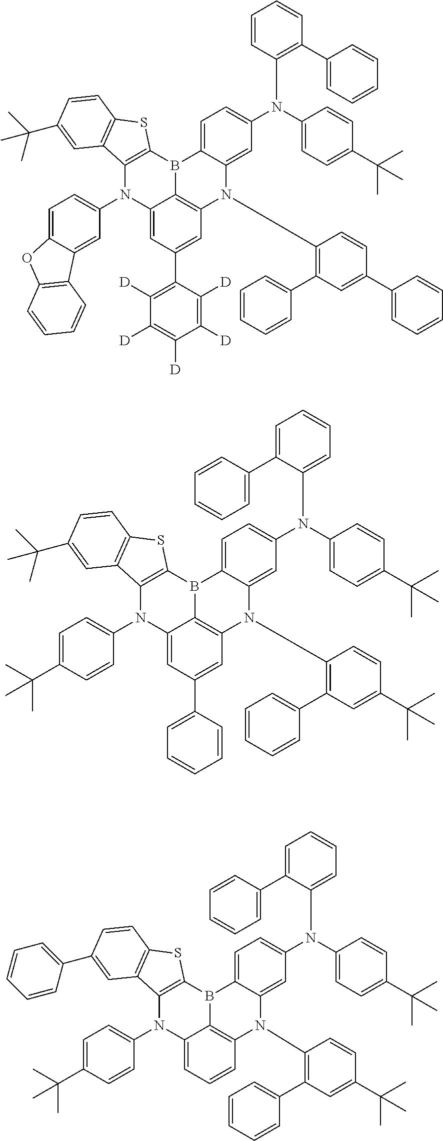 [SMILES: [2H]c1c([2H])c([2H])c(-c2cc3c4c(c2)N(c2ccc5oc6ccccc6c5c2)c2c(sc5ccc(C(C)(C)C)cc25)B4c2ccc(N(c4ccc(C(C)(C)C)cc4)c4ccccc4-c4ccccc4)cc2N3c2ccc(-c3ccccc3)cc2-c2ccccc2)c([2H])c1[2H] ; CC(C)(C)c1ccc(N(c2ccc3c(c2)N(c2ccc(C(C)(C)C)cc2-c2ccccc2)c2cc(-c4ccccc4)cc4c2B3c2sc3ccc(C(C)(C)C)cc3c2N4c2ccc(C(C)(C)C)cc2)c2ccccc2-c2ccccc2)cc1 ; CC(C)(C)c1ccc(N(c2ccc3c(c2)N(c2ccc(C(C)(C)C)cc2-c2ccccc2)c2cccc4c2B3c2sc3ccc(-c5ccccc5)cc3c2N4c2ccc(C(C)(C)C)cc2)c2ccccc2-c2ccccc2)cc1]

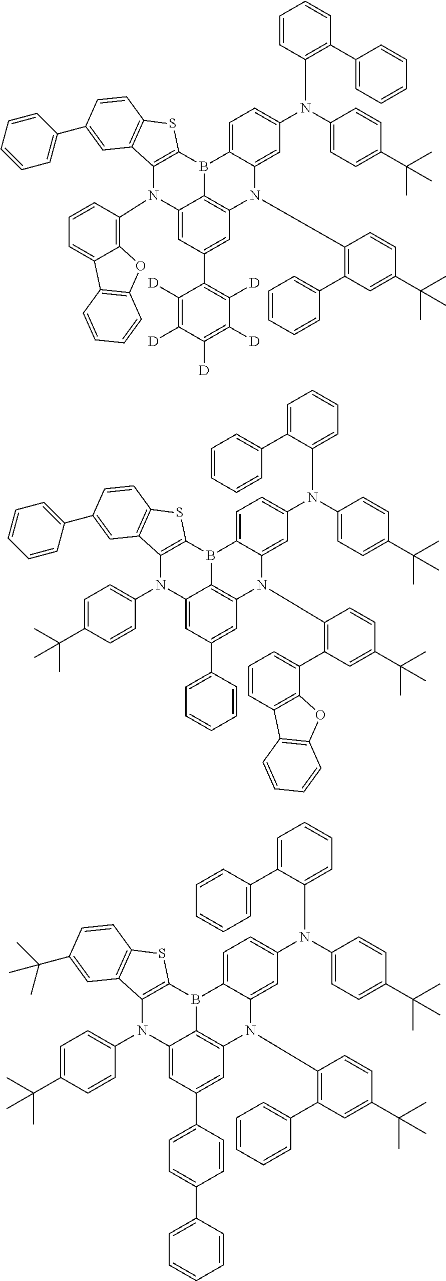

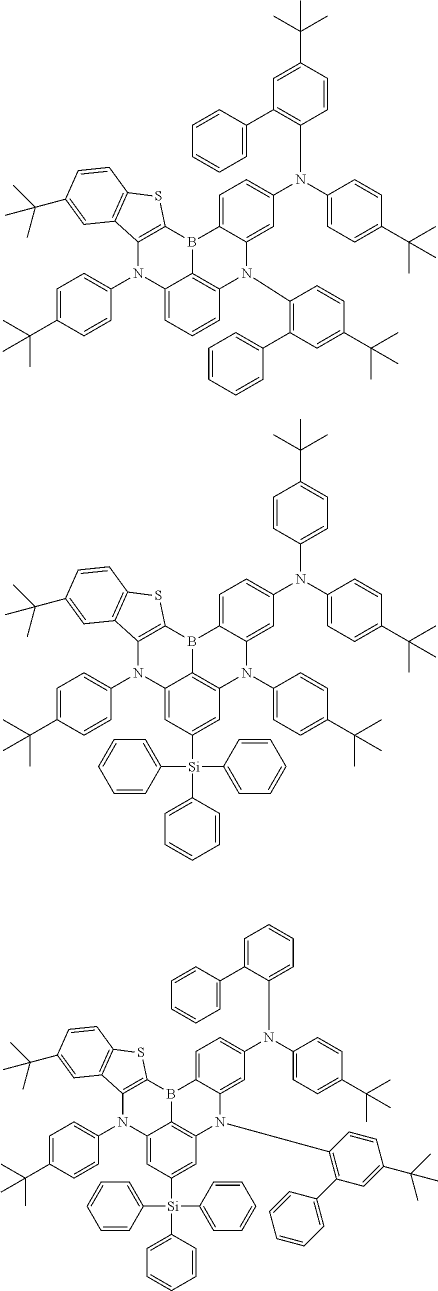

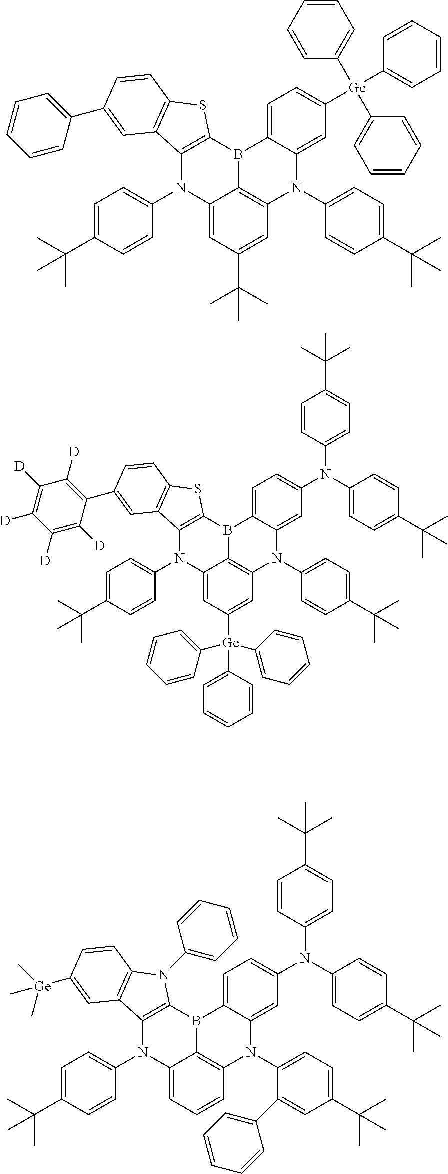

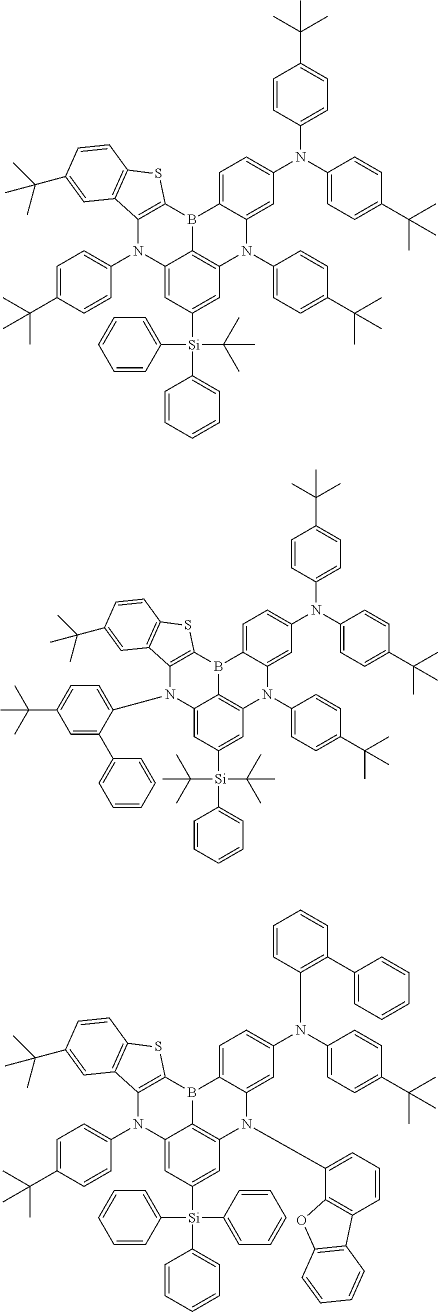

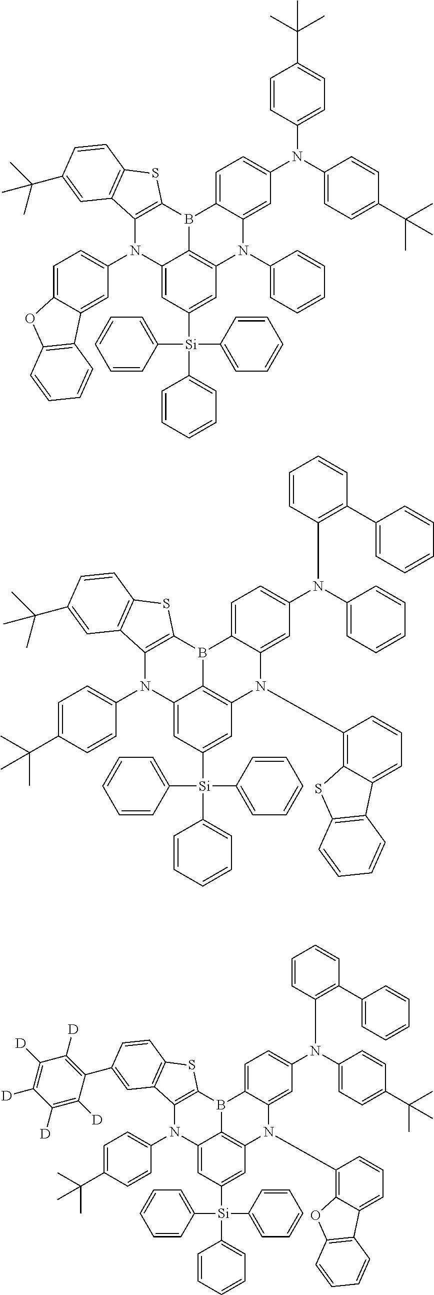

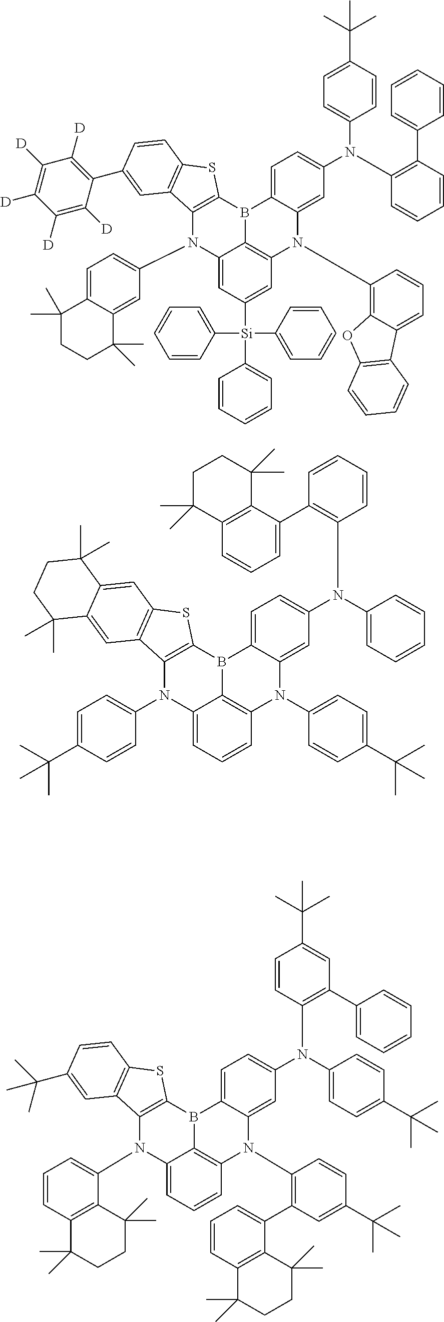

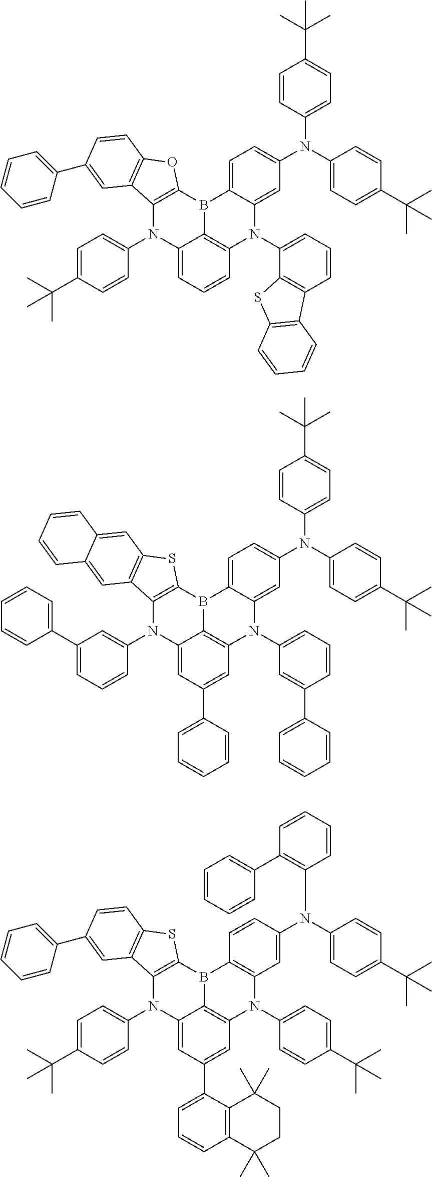

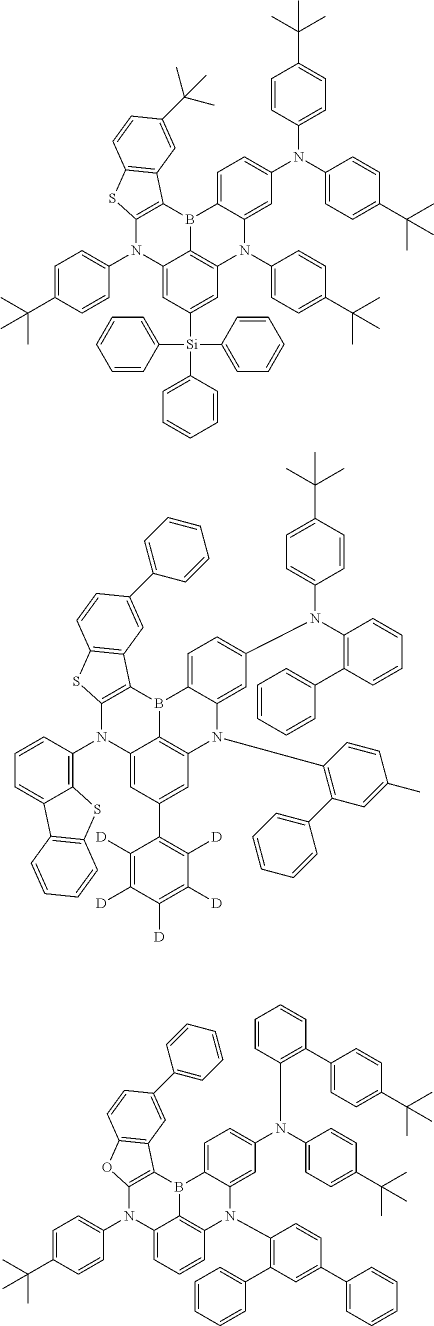

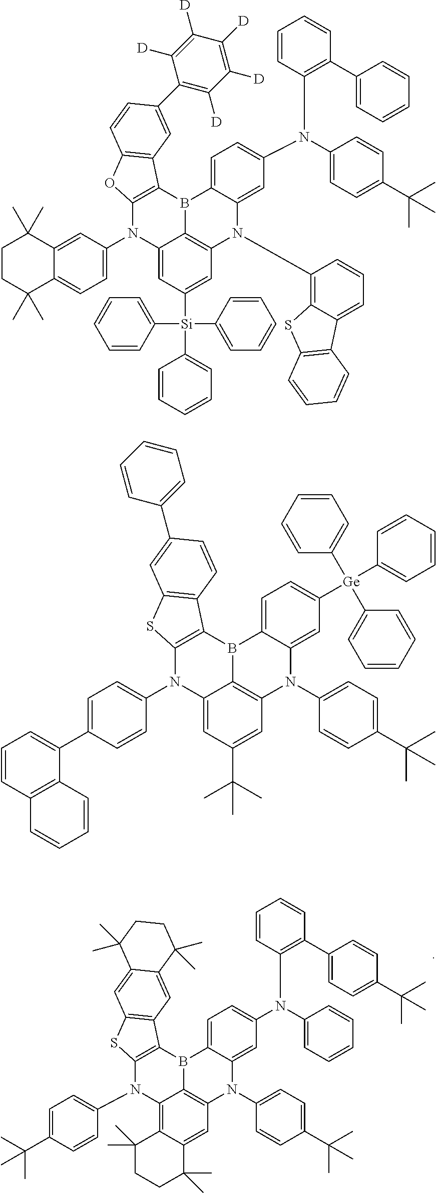

11. The organic light emitting device according to claim 7, wherein the organic layers comprise a hole injecting layer, a hole transport layer, an electron blocking layer, a functional layer having functions of both hole injection and hole transport, a light emitting layer, an electron transport layer, an electron injecting layer, a hole blocking layer, and/or a functional layer having functions of both electron injection and electron transport.

12. The organic light emitting device according to claim 11, wherein each of the organic layers is formed by a deposition or solution process.

13. The organic light emitting device according to claim 7, wherein one or more hosts other than the anthracene derivative represented by Formula 1 are mixed or stacked in the light emitting layer.

14. The organic light emitting device according to claim 7, wherein one or more dopants other than the polycyclic compound represented by Formula 2 are mixed or stacked in the light emitting layer.

15. The organic light emitting device according to claim 7, wherein the organic light emitting device is used in a display or lighting system selected from flat panel displays, flexible displays, monochromatic flat panel lighting systems, white flat panel lighting systems, flexible monochromatic lighting systems, flexible white lighting systems, displays for automotive applications, displays for virtual reality, and displays for augmented reality.
